(19) United States
(12) Patent Application Publication
(10) Pub. No.: US 2020/0181274 A1
(43) Pub. Date: Jun. 11, 2020

(54) BISPECIFIC ANTIBODIES THAT BIND CD 123 CD3

(71) Applicant: NOVARTIS AG, Basel (CH)

(72) Inventors: Michael Wayne SAVILLE, Monrovia, CA (US); Paul FOSTER, Monrovia, CA (US)

(21) Appl. No.: 16/617,675

(22) PCT Filed: Jun. 1, 2018
(86) PCT No.: PCT/US2018/035613

§ 371 Date: Nov. 27, 2019

Related U.S. Application Data
(60) Provisional application No. 62/513,763, filed on Jun. 1, 2017.

(51) Int. Cl. C07K 16/28

(52) U.S. Cl. CPC C07K 16/2866; C07K 16/2809; A61K 2039/545; C07K 16/2827; C07K 16/2818

(57) ABSTRACT

The present invention is directed to novel bispecific anti-CD 123×anti-CD3 antibodies.

### CROSS-REFERENCE TO RELATED APPLICATIONS

This application claims priority under 35 U.S.C. § 119(e) to U.S. Provisional Patent Application No. 62/513,763, filed Jun. 1, 2017, which is expressly incorporated herein by reference in its entirety, with particular reference to the figures, legends and claims therein.

### BACKGROUND OF THE INVENTION

Antibody-based therapeutics have been used successfully to treat a variety of diseases, including cancer and autoimmune/inflammatory disorders. Yet improvements to this class of drugs are still needed, particularly with respect to enhancing their clinical efficacy. One avenue being explored is the engineering of additional and novel antigen binding sites into antibody-based drugs such that a single immunoglobulin molecule co-engages two different antigens. Because the considerable diversity of the antibody variable region (Fv) makes it possible to produce an Fv that recognizes virtually any molecule, the typical approach to the generation of such bispecific antibodies is the introduction of new variable regions into the antibody.

A number of alternate antibody formats have been explored for bispecific targeting (Chames & Baty, 2009, mAbs 1[6]:1-9; Holliger & Hudson, 2005, Nature Biotechnology 23[9]:1126-1136; Kontermann, mAbs 4(2):182 (2012), all of which are expressly incorporated herein by reference). Initially, bispecific antibodies were made by fusing two cell lines that each produced a single monoclonal antibody (Milstein et al., 1983, Nature 305:537-540). Although the resulting hybrid hybridoma or quadroma did produce bispecific antibodies, they were only a minor population, and extensive purification was required to isolate the desired antibody. An engineering solution to this was the use of antibody fragments to make bispecifics. Because such fragments lack the complex quaternary structure of a full length antibody, variable light and heavy chains can be linked in single genetic constructs. Antibody fragments of many different forms have been generated, including diabodies, single chain diabodies, tandem scFvs, and Fab2 bispecifics (Chames & Baty, 2009, mAbs 1[6]:1-9; Holliger & Hudson, 2005, Nature Biotechnology 23[9]:1126-1136; expressly incorporated herein by reference). While these formats can be expressed at high levels in bacteria and may have favorable penetration benefits due to their small size, they clear rapidly in vivo and can present manufacturing obstacles related to their production and stability. A principal cause of these drawbacks is that antibody fragments typically lack the constant region of the antibody with its associated functional properties, including larger size, high stability, and binding to various Fc receptors and ligands that maintain long half-life in serum (i.e. the neonatal Fc receptor FcRn) or serve as binding sites for purification (i.e. protein A and protein G).

More recent work has attempted to address the shortcomings of fragment-based bispecifics by engineering dual binding into full length antibody-like formats (Wu et al., 2007, Nature Biotechnology 25[11]:1290-1297; U.S. Ser. No. 12/477,711; Michaelson et al., 2009, mAbs 1[2]:128-141; PCT/US2008/074693; Zuo et al., 2000, Protein Engineering 13[5]:361-367; U.S. Ser. No. 09/865,198; Shen et al., 2006, J Biol Chem 281[16]:10706-10714; Lu et al., 2005, J Biol Chem 280[20]:19665-19672; PCT/US2005/025472; expressly incorporated herein by reference). These formats overcome some of the obstacles of the antibody fragment bispecifics, principally because they contain an Fc region. One significant drawback of these formats is that, because they build new antigen binding sites on top of the homodimeric constant chains, binding to the new antigen is always bivalent.

For many antigens that are attractive as co-targets in a therapeutic bispecific format, the desired binding is monovalent rather than bivalent. For many immune receptors, cellular activation is accomplished by cross-linking of a monovalent binding interaction. The mechanism of cross-linking is typically mediated by antibody/antigen immune complexes, or via effector cell to target cell engagement. For example, the low affinity Fc gamma receptors (FcγRs) such as FcγRIIa, FcγRIIb, and FcγRIIIa bind monovalently to the antibody Fc region. Monovalent binding does not activate cells expressing these FcγRs; however, upon immune complexation or cell-to-cell contact, receptors are cross-linked and clustered on the cell surface, leading to activation. For receptors responsible for mediating cellular killing, for example FcγRIIIa on natural killer (NK) cells, receptor cross-linking and cellular activation occurs when the effector cell engages the target cell in a highly avid format (Bowles & Weiner, 2005, J Immunol Methods 304:88-99, expressly incorporated by reference). Similarly, on B cells the inhibitory receptor FcγRIIb downregulates B cell activation only when it engages into an immune complex with the cell surface B-cell receptor (BCR), a mechanism that is mediated by immune complexation of soluble IgG's with the same antigen that is recognized by the BCR (Heyman 2003, Immunol Lett 88[2]:157-161; Smith and Clatworthy, 2010, Nature Reviews Immunology 10:328-343; expressly incorporated by reference). As another example, CD3 activation of T-cells occurs only when its associated T-cell receptor (TCR) engages antigen-loaded MHC on antigen presenting cells in a highly avid cell-to-cell synapse (Kuhns et al., 2006, Immunity 24:133-139). Indeed nonspecific bivalent cross-linking of CD3 using an anti-CD3 antibody elicits a cytokine storm and toxicity (Perruche et al., 2009, J Immunol 183[2]:953-61; Chatenoud & Bluestone, 2007, Nature Reviews Immunology 7:622-632; expressly incorporated by reference). Thus for practical clinical use, the preferred mode of CD3 co-engagement for redirected killing of target cells is monovalent binding that results in activation only upon engagement with the co-engaged target.

CD123, also known as interleukin-3 receptor alpha (IL-3Ra), is expressed on dendritic cells, monocytes, eosinophils and basophils. CD123 is also constitutively expressed by committed hematopoietic stem/progenitor cells, by most of the myeloid lineage (CD13+, CD14+, CD33+, CD15_{low}), and by some CD19+ cells. It is absent from CD3+ cells.

Accordingly, there is a need for improved bispecific anti-CD123×anti-CD3 antibodies and the use of such antibodies for use in therapy.

### BRIEF SUMMARY OF THE INVENTION

In one aspect, the invention provides a method for treating a CD123-expressing cancer in a subject, comprising administering to the subject having the CD123-expressing cancer an intravenous dose of a bispecific anti-CD123×anti-CD3 antibody, for a time period sufficient to treat the CD123-expressing cancer, in combination with at least one other therapeutic agent, wherein at least one of the other therapeutic agent is selected from the group consisting of PD1 inhibitors, PDL1 inhibitors, PDL2 inhibitors, TIM3 inhibitors, LAG3 inhibitors, CTLA4 inhibitors, TIGIT inhibitors, BTLA inhibitors, CD47 inhibitors, IDO inhibitors, GITR agonists, and ICOS agonists.

In an exemplary embodiment, the CD123-expressing cancer is a hematologic cancer. In an exemplary embodiment, the CD123-expressing cancer is leukemia.

In an exemplary embodiment, the bispecific anti-CD123×anti-CD3 antibody comprises: a) a first monomer comprising SEQ ID NO: 1; b) a second monomer comprising SEQ ID NO: 2; and c) a light chain comprising SEQ ID NO: 3. In an exemplary embodiment, the bispecific anti-CD123×anti-CD3 antibody comprises: a) an anti-CD123 variable heavy (VH) domain comprising SEQ ID NO: 19; b) an anti-CD123 variable light (VL) domain comprising SEQ ID NO: 20; c) an anti-CD3 variable heavy (VH) domain comprising SEQ ID NO: 21; and d) an anti-CD3 variable light (VL) domain comprising SEQ ID NO: 22. In an exemplary embodiment, the bispecific anti-CD123×anti-CD3 antibody comprises a) an anti-CD3 VH domain comprising a VHCDR1 comprising SEQ ID NO: 23, a VHCDR2 comprising SEQ ID NO: 24 and a VHCDR3 comprising SEQ ID NO: 25; b) an anti-CD3 VL domain comprising a VLCDR1 comprising SEQ ID NO: 26, a VLCDR2 comprising SEQ ID NO: 27 and a VLCDR3 comprising SEQ ID NO: 28; c) an anti-CD123 VH domain comprising a VHCDR1 comprising SEQ ID NO: 29, a VHCDR2 comprising SEQ ID NO: 30 and a VHCDR3 comprising SEQ ID NO: 31; d) an anti-CD123 VL domain comprising a VLCDR1 comprising SEQ ID NO: 32, a VLCDR2 comprising SEQ ID NO: 33 and a VLCDR3 comprising SEQ ID NO: 34. In an exemplary embodiment, the bispecific anti-CD123×anti-CD3 antibody is XmAb14045.

In an exemplary embodiment, the at least one of the other therapeutic agents is a PD1 inhibitor. In an exemplary embodiment, the PD1 inhibitor is an anti-PD1 antibody. In an exemplary embodiment, the anti-PD1 antibody is selected from the group consisting of nivolumab (Opdivo®), pembrolizumab (Keytruda®), pidilizumab (Medivation/Pfizer), spartalizumab, JNJ-63723283 (J&J), TSR-042 (Tesaro), cemiplimab (Sanofi), AMP-224 (Amplimmune/GSK), MEDI0680 (AstraZeneca), MGA012 (MacroGenics/Incyte), MGD013 (MacroGenics), MGD019 (MacroGenics), SHR-1210 (Shanghai Hengrui Pharma/Incyte), GLS-010 (Gloria Pharma/WuXi Biologics), JS001 (Shanghai Junshi Biosciences), tislelizumab (BeiGene/Celgene), sintilimab (Innovent), CX-188 (CytomX Therapeutics), and CS1003 (CStone Pharmaceuticals). In an exemplary embodiment, the anti-PD1 antibody is selected from the group consisting of nivolumab (Opdivo®; BMS), pembrolizumab (Keytruda®; Merck), and pidilizumab (Medivation/Pfizer). In an exemplary embodiment, the anti-PD1 antibody is spartalizumab. In an exemplary embodiment, the at least one of the other therapeutic agents is a PDL1 inhibitor. In an exemplary embodiment, the PDL1 inhibitor is an anti-PDL1 antibody. In an exemplary embodiment, the anti-PDL1 antibody is selected from the group consisting of atezolizumab (Tecentriq®; Genentech/Roche), avelumab (Bavencio®; EMD Serono), durvalumab (Imfinzi®; Medlmmune/AstraZeneca), FAZ053, LY3300054 (Lilly), ABBV-181 (AbbVie), MSB2311 (MabSpace Biosciences), BMS-936559, CS1001 (CStone Pharmaceuticals), KNO35 (Alphamab), CA-327 (Curis), CX-072 (CytomX Therapeutics), M7824 (EMD Serono), HTI-1316 (Hengrui Therapeutics), and JS003 (Shanghai Junshi Biosciences). In an exemplary embodiment, the at least one other therapeutic agent further comprises a chemotherapeutic. In an exemplary embodiment, the chemotherapeutic is selected from the group consisting of alkylating agents, anti-metabolites, kinase inhibitors, proteasome inhibitors, vinca alkaloids, anthracyclines, antitumor antibiotics, aromatase inhibitors, topoisomerase inhibitors, mTOR inhibitors, retinoids, and combinations thereof. In an exemplary embodiment, the at least one other therapeutic agent further comprises a side-effect ameliorating agent. In an exemplary embodiment, the side-effect ameliorating agent is selected from the group consisting of: a steroid, an antihistamine, anti-allergic agents, antinausea agents (or anti-emetics), analgesic agent, antipyretic agent, cytoprotective agents, vasopressor agents, anticonvulsant agent, TNFα inhibitor, IL6 inhibitor, and combinations thereof. In an exemplary embodiment, the side-effect ameliorating agent is selected from the group consisting of corticosteroids, TNFα inhibitors, IL-1R inhibitors, and IL-6 inhibitors wherein said side-effect ameliorating agent is a combination of a corticosteroid, Benadryl® and Tylenol®, wherein said corticosteroid, Benadryl® and Tylenol® are administered to said human subject prior to the administration of said bispecific anti-CD123×anti-CD3 antibody. In an exemplary embodiment, the bispecific anti-CD123×anti-CD3 antibody and the at least one other therapeutic agent are administered concurrently. In an exemplary embodiment, the administration of the at least one other therapeutic agent begins before the administration of the bispecific anti-CD123×anti-CD3 antibody.

In an exemplary embodiment, the subject is a mammal. In an exemplary embodiment, the subject is a human subject.

In one aspect, the intravenous dose according to the present invention is administered to a human subject between about 1 hour and about 3 hours. In some embodiments, the time period sufficient to treat a CD123-expressing cancer, e.g., a hematologic cancer, e.g., leukemia in a human subject is between about 3 weeks and 9 weeks. In some embodiments, the time period sufficient to treat a CD123-expressing cancer, e.g., a hematologic cancer, e.g., leukemia in a human subject is between about 4 weeks and 9 weeks.

In one aspect, the bispecific anti-CD123×anti-CD3 antibody according to the present invention is XmAb14045 as described herein. In such embodiments, the XmAb14045 bispecific anti-CD123×anti-CD3 antibody includes a first monomer comprising SEQ ID NO: 1, a second monomer comprising SEQ ID NO: 2, and a light chain comprising SEQ ID NO: 3.

In an exemplary embodiment, the CD123-expressing cancer is a hematologic cancer. In an exemplary embodiment, the CD123-expressing cancer is leukemia.

In one aspect, a human subject that is being treated according to the present invention has leukemia, for example, leukemia selected from the group consisting of acute myeloid leukemia (AML), chronic myeloid leukemia (CML), acute lymphocytic leukemia (ALL), blastic plasmacytoid dendritic cell neoplasm, and hairy cell leukemia (HCL). In some embodiments, leukemia is acute myeloid leukemia (AML). In some embodiments, AML is blastic plasmacytoid dendritic cell neoplasm (BPDCN). In some embodiments, leukemia is ALL. In some embodiments, ALL is B-cell acute lymphocytic leukemia (B-ALL).

In one aspect, the methods and antibodies of the present invention further comprise, prior to the administering, assessing the weight of the human subject.

In some embodiments, the methods and antibodies of the present invention further comprise, prior to the administering of a bispecific anti-CD123×anti-CD3 antibody (e.g., XmAb14045), administering a steroid to the human subject. In some embodiments, the methods of the present invention further comprise, prior to the administering of a bispecific anti-CD123×anti-CD3 antibody, assessing the weight of the human subject. In some embodiments, the methods of the present invention further comprise administering to the human subject a checkpoint inhibitor or agonists, for example, an inhibitor of PD1, PDL1, TIM3, LAG3, CTLA4, TIGIT, or BTLA or an agonist of ICOS.

In an exemplary embodiment, the present invention provides a method for treating a CD123-expressing cancer, e.g., a hematologic cancer, e.g., leukemia, in a subject, comprising: administering to the human subject having a CD123-expressing cancer, e.g., a hematologic cancer, e.g., leukemia, an intravenous dose of between about 1 ng/kg and about 800 ng/kg of a bispecific anti-CD123×anti-CD3 antibody (e.g., XmAb14045) once every 6-8 days for a time period sufficient to treat the CD123-expressing cancer.

In some embodiments, the methods and antibodies of the present invention further comprise administering to the subject another therapy. In one aspect, the methods and antibodies of the present invention further comprise administering to said subject one or more other therapies.

### BRIEF DESCRIPTION OF THE DRAWINGS

FIG. 1 depicts a particularly useful bispecific format of the invention, referred to as a “bottle opener”, which is also the format of XmAb14045. It should be noted that the scFv and Fab domains can be switched (e.g. anti-CD3 as a Fab, and anti-CD123 as a scFv).

FIG. 2 depicts the sequences of the three polypeptide chains that make up XmAb14045, an anti-CD123×anti-CD3 antibody of particular use in the present invention. The CDRs are underlined and the junction between domains is denoted by a slash (“/”). The charged scFv linker is double underlined; as will be appreciated by those in the art, the linker may be substituted with other linkers, and particularly other charged linkers that are depicted in FIG. 7 of US Publication Number 2014/0288275, or other non-charged linkers (SEQ ID NO:441 of US Publication Number 2014/0288275).

FIG. 3 depicts the engineering of a number of anti-CD123 Fab constructs to increase affinity to human CD123 and stability of the 7G3 H1L1 construct, including the amino acid changes.

FIG. 4 depicts the properties of final affinity and stability optimized humanized variants of the parental 7G3 murine antibody.

FIG. 5A-5B depicts additional anti-CD123 Fab sequences of the invention, with the CDRs underlined.

FIG. 6 depicts additional anti-CD123×anti CD3 sequences of the invention. The CDRs are underlined and the junction between domains is denoted by a slash (“/”). The charged scFv linker is double underlined; as will be appreciated by those in the art, the linker may be substituted with other linkers, and particularly other charged linkers that are depicted in FIG. 7 of US Publication Number 2014/0288275, or other non-charged linkers (SEQ ID NO:441 of US Publication Number 2014/0288275).

FIG. 7A-7D depicts additional bispecific formats of use in the present invention, as are generally described in FIG. 1 and the accompanying Legend and supporting text of U.S. Ser. No. 14/952,714 (incorporated herein by reference).

FIG. 8 depicts RTCC with intact or T cell depleted PBMC against KG-1a target cells. Effector cells (400k), intact or magnetically-depleted PBMC were incubated with carboxyfluorescein succinimidyl ester-labeled KG-1a target cells (10k) for 24 hours and stained with annexin V for cell death.

FIG. 9 depicts CD123hiCD33hi depletion over a dose range of XmAb14045 in AML human subject PBMC. Five AML human subject PBMC samples were incubated with a dose range of XmAb14045 (0.12 to 90 ng/mL) for 6 days, and live cells were gated to count CD123hiCD33hi target cells. The lowest concentration (0.04 ng/mL) point is the no drug control for plotting on logarithmic scale. Each point is normalized to account for cell count variability.

FIG. 10 depicts Ki67 levels in T cells from AML human subject PBMC with XmAb14045. Five AML human subject PBMC samples were incubated with a dose range of XmAb14045 (0.12 to 90 ng/mL) for 6 days, and live cells were gated for CD4+ and CD8+ T cells to count Ki67+ cells. The lowest concentration (0.04 ng/mL) point is the no drug control, for plotting on a logarithmic scale.

FIG. 11 depicts number of AML blasts in human subject PBMCs treated with XmAb14045. PBMC from a single AML human subject was incubated with 9 or 90 ng/mL XmAb14045 for 24 or 48 hours and blast counts were plotted. Normal donor PBMCs were also used as a control.

FIG. 12 depicts leukemic blast cells in AML human subject PBMC. PBMCs from six AML human subjects were incubated with antibodies for 48 hours and blasts were counted and plotted. One donor (AML #1) did not have XENP13245 treatment and each line is a single donor.

FIG. 13 depicts KG-1a tumor cell apoptosis with AML PBMC. Carboxyfluorescein succinimidyl ester-labeled CD123+ KG-1a cells were added to the PBMC to examine target cell cytotoxicity stimulated by the AML effector T cells. Staining with the apoptosis marker annexin-V was used to detect KG-1a cell death after 48 hours of incubation.

FIG. 14 depicts effect of XmAb14045 on tumor burden over time in a mouse xenograft model of AML.

FIG. 15 depicts reduction of tumor burden after 3 weekly doses of XmAb14045.

FIG. 16 depicts effect of XmAb14045 on T cell number in a mouse xenograft model of AML. Peripheral blood CD45+CD8+ events by flow cytometry. Samples taken on Day 11 and 20 after XmAb14045 administration.

### DETAILED DESCRIPTION OF THE INVENTION

### I. Definitions

In order that the application may be more completely understood, several definitions are set forth below. Such definitions are meant to encompass grammatical equivalents.

By “CD3” or “cluster of differentiation 3” herein is meant a T-cell co-receptor that helps in activation of both cytotoxic T-cell (e.g., CD8+naïve T cells) and T helper cells (e.g., CD4+naïve T cells) and is composed of four distinct chains: one CD3γ chain (e.g., Genbank Accession Numbers NM 000073 and MP 000064 (human)), one CD3δ chain (e.g., Genbank Accession Numbers NM 000732, NM 001040651, NP 00732 and NP 001035741 (human)), and two CD3ε chains (e.g., Genbank Accession Numbers NM 000733 and NP 00724 (human)). The chains of CD3 are highly related cell-surface proteins of the immunoglobulin superfamily containing a single extracellular immunoglobulin domain. The CD3 molecule associates with the T-cell receptor (TCR) and -chain to form the T-cell receptor (TCR) complex, which functions in generating activation signals in T lymphocytes.

By “CD123” or “Cluster of Differentiation 123” or “CD123 antigen” or “interleukin-3 receptor alpha” or “IL3RA” or “interleukin3 receptor subunit alpha” is meant the interleukin 3 specific subunit of a type I heterodimeric cytokine receptor (e.g., Genbank Accession Numbers NM 001267713, NM 002183, NP 001254642 and NP 002174 (human)). CD123 interacts with a signal transducing beta subunit to form interleukin-3 receptor, which helps in the transmission of interleukin 3. CD123 is found on pluripotent progenitor cells and induces tyrosine phosphorylation within the cell and promotes proliferation and differentiation within the hematopoietic cell lines. CD123 is expressed across acute myeloid leukemia (AML subtypes, including leukemic stem cells

By “bispecific” or “bispecific antibody” herein is meant any non-native or alternate antibody formats, including those described herein, that engage two different antigens (e.g., CD3×CD123 bispecific antibodies).

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233# designates a deletion of glutamic acid at position 233. Additionally, EDA233- or EDA233# designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example, N434S or 434S is an Fc variant with the substitution serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e. “analogs”, such as peptoids (see Simon et al., PNAS USA 89(20):9367 (1992), entirely incorporated by reference). The amino acids may either be naturally occurring or synthetic (e.g. not an amino acid that is coded for by DNA); as will be appreciated by those in the art. For example, homo-phenylalanine, citrulline, ornithine and noreleucine are considered synthetic amino acids for the purposes of the invention, and both D- and L-(R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

By “amino acid” and “amino acid identity” as used herein is meant one of the 20 naturally occurring amino acids that are coded for by DNA and RNA.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcqammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

By “heavy constant region” herein is meant the CH1-hinge-CH2-CH3 portion of an antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. The two target antigens of the present invention are human CD3 and human CD123.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g. making the pI higher) then steric variants that are “charge pairs” that can be utilized as well do not interfere with the pI variants, e.g. the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer that incorporates one set of the pair will go, such that pI separation is maximized using the pI of the skews as well.

By “target cell” as used herein is meant a cell that expresses a target antigen.

By “variable region” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10-4 M, at least about 10-5 M, at least about 10-6 M, at least about 10-7 M, at least about 10-8 M, at least about 10-9 M, alternatively at least about 10-10 M, at least about 10-11 M, at least about 10-12 M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay.

As used herein, the term “target activity” refers to a biological activity capable of being modulated by a selective modulator. Certain exemplary target activities include, but are not limited to, binding affinity, signal transduction, enzymatic activity, tumor growth, effects on particular biomarkers related to CD123 disorder pathology.

By “refractory” in the context of a cancer is intended the particular cancer is resistant to, or non-responsive to, therapy with a particular therapeutic agent. A cancer can be refractory to therapy with a particular therapeutic agent either from the onset of treatment with the particular therapeutic agent (i.e., non-responsive to initial exposure to the therapeutic agent), or as a result of developing resistance to the therapeutic agent, either over the course of a first treatment period with the therapeutic agent or during a subsequent treatment period with the therapeutic agent.

As used herein, the IC₅₀ refers to an amount, concentration or dosage of a particular test compound that achieves a 50% inhibition of a maximal response, such as inhibition of the biological activity of CD123, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

### II. Overview

In one aspect, the invention provides a method for treating a CD123-expressing cancer in a subject, comprising administering to the subject having the CD123-expressing cancer an intravenous dose of a bispecific anti-CD123×anti-CD3 antibody, for a time period sufficient to treat the CD123-expressing cancer, in combination with at least one other therapeutic agent described herein.

In one aspect, the invention provides a method for treating a CD123-expressing cancer in a subject, comprising administering to the subject having the CD123-expressing cancer an intravenous dose of a bispecific anti-CD123×anti-CD3 antibody, for a time period sufficient to treat the CD123-expressing cancer, in combination with at least one other chemotherapeutic agent described herein. In one aspect, the invention provides a method for treating a CD123-expressing cancer in a subject, comprising administering to the subject having the CD123-expressing cancer an intravenous dose of a bispecific anti-CD123×anti-CD3 antibody, for a time period sufficient to treat the CD123-expressing cancer, in combination with at least one other side-effect ameliorating agent described herein.

The invention provides methods of treating a cancer that include cells expressing CD123 (“CD123-expressing cancer”), for example, a hematologic cancer, such as leukemia, through the administration of certain bispecific anti-CD123×anti-CD3 antibodies at particular dosages in combination with another therapy. These particular dosages are reduced over those known in the art. The present invention also provides methods of combination therapies, for example, methods of treating a cancer that include cells expressing CD123 (“CD123-expressing cancer”), e.g., a hematologic cancer, such as leukemia, through the administration of certain bispecific anti-CD123×anti-CD3 antibodies (e.g., XmAb14045) in combination with one or more checkpoint inhibitors or agonists, such as an inhibitor of PD1, PDL1, TIM3, LAG3, CTLA4, TIGIT, or BTLA or an agonist of ICOS.

### III. Antibodies

The present invention is directed to the administration of bispecific anti-CD123×anti-CD3 antibodies for the treatment of particular leukemias as outlined herein, as outlined in PCT Application Nos. PCT/US15/62772 (WO2016/086189), PCT/US16/29797 (WO2016/182751), as well as U.S. Ser. Nos. 14/952,714, 15/141,350, 15/186,167, 62/085,117, 62/085,027, 62/084,908, 62/085,106, 62/159,111, 62/251,005, and 62/250,971, all of which are expressly incorporated herein by reference, particularly for the bispecific formats of the figures, as well as all sequences, Figures and accompanying Legends therein.

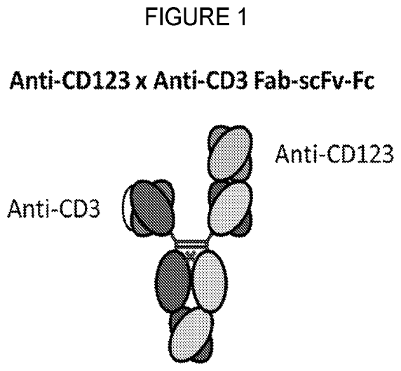

In some embodiments, the bispecific anti-CD123×anti-CD3 antibodies have a “bottle opener” format as is generally depicted in FIG. 1. In this embodiment, the anti-CD3 antigen binding domain is the scFv-Fc domain monomer and the anti-CD123 antigen binding domain is the Fab monomer (terms as used in US Publication Nos. 2014/0288275 and 2014-0294823 as well as in U.S. Ser. No. 15/141,350, all of which are expressly incorporated by reference in their entirety and specifically for all the definitions, sequences of anti-CD3 antigen binding domains and sequences of anti-CD123 antigen binding domains).

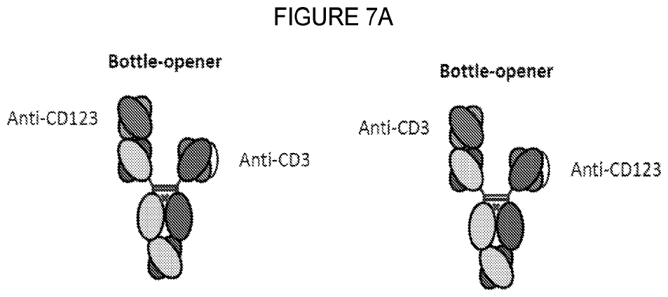

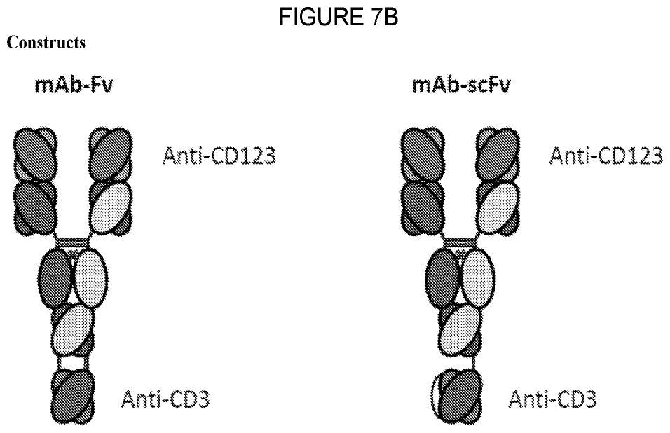

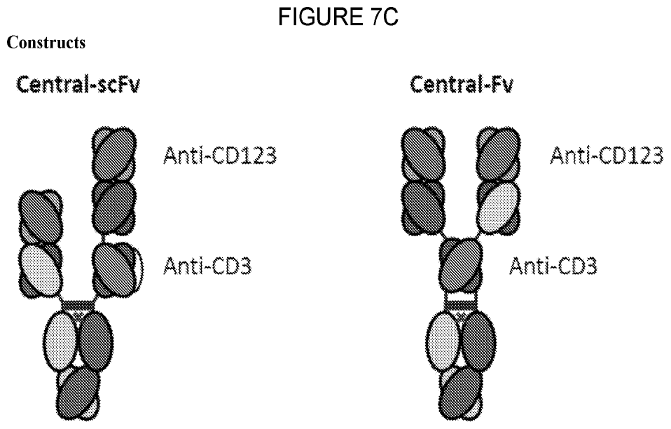

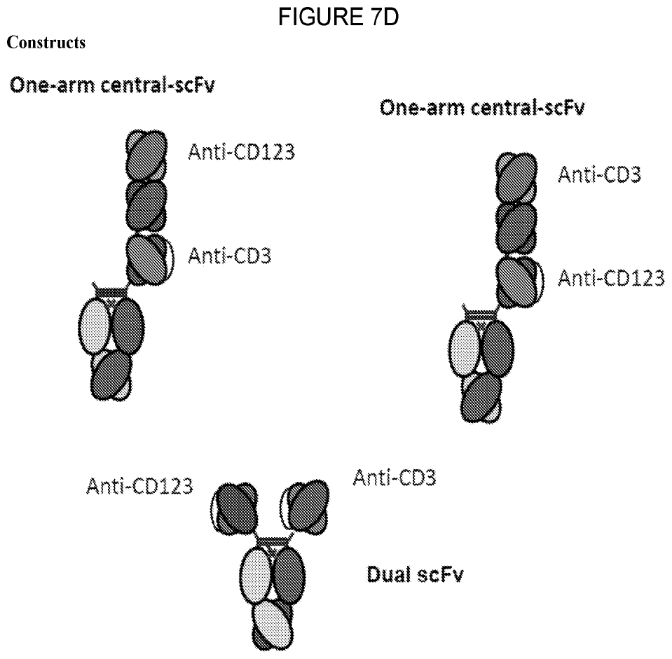

Alternate formats for the bispecific, heterodimeric anti-CD123×anti-CD3 antibodies of the invention are shown in FIG. 7, which also generally rely on the use of Fabs and scFv domains in different formats.

In addition, it is also possible to make non-heterodimeric anti-CD123×anti-CD3 bispecific antibodies as are known in the art, that can be dosed at the same dosage levels as described herein for the heterodimeric bispecific anti-CD123×anti-CD3 antibodies.

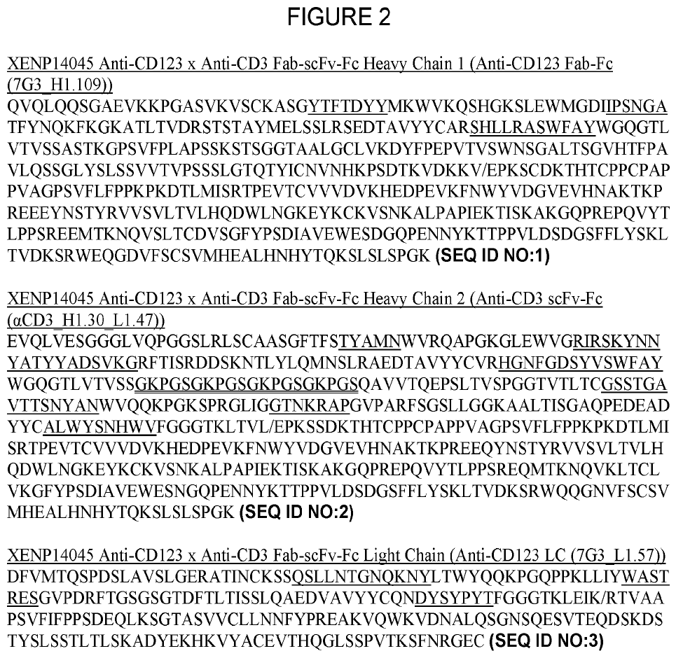

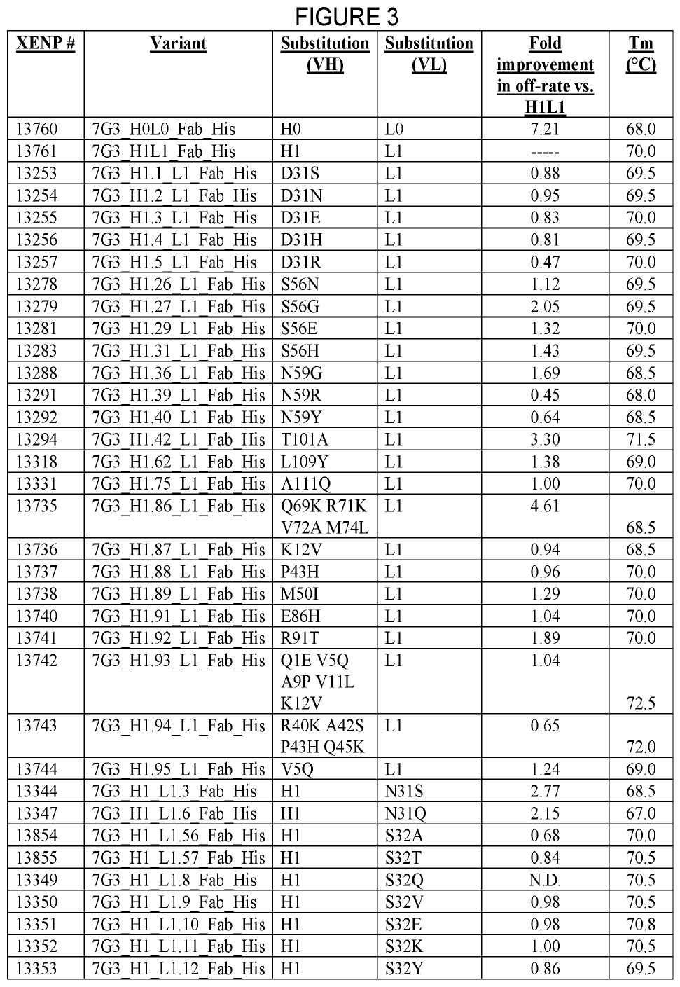

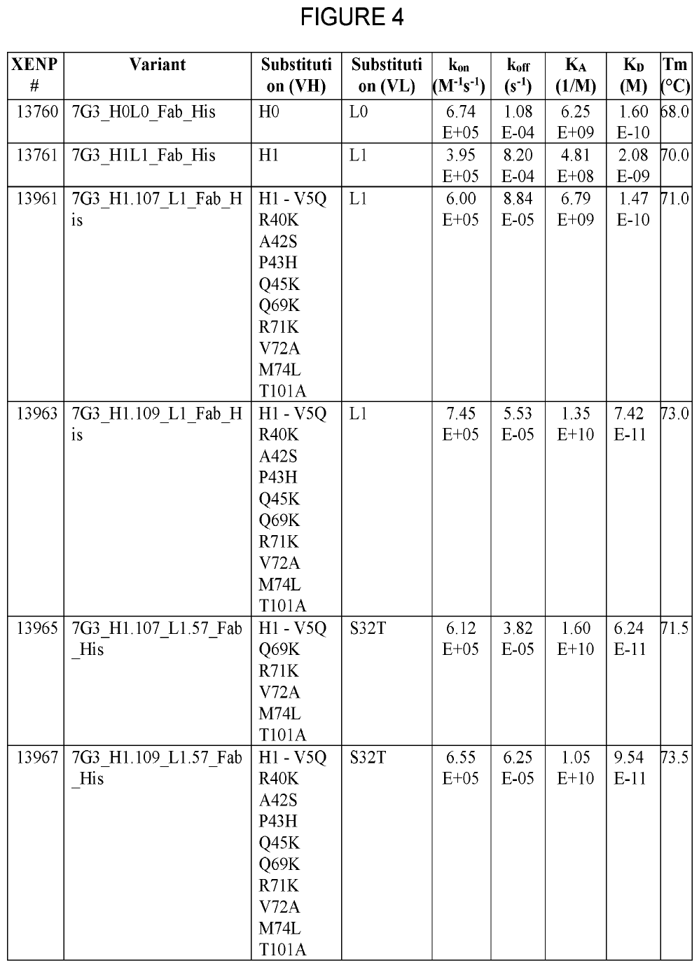

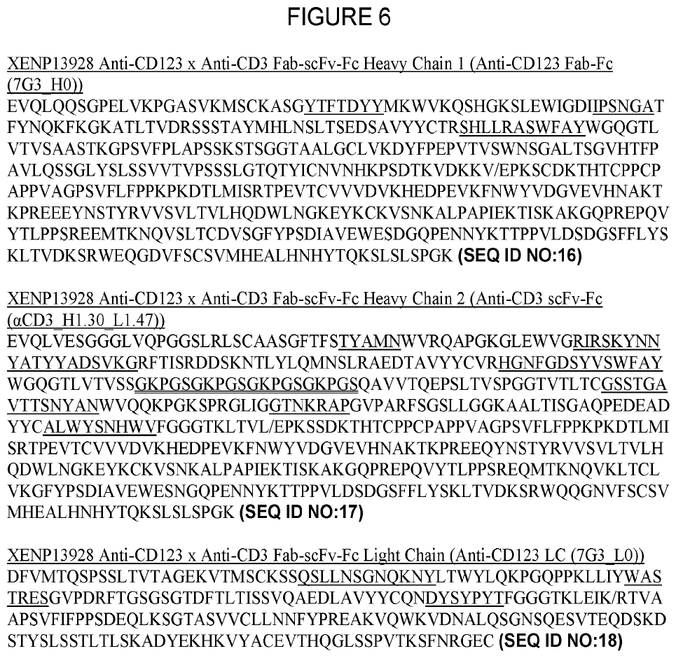

The anti-CD3 scFv antigen binding domain can have the sequence depicted in FIG. 2, or can be selected from:
1) the set of 6 CDRs (vhCDR1, vhCDR2, vhCDR3, vlCDR1, vlCDR2 and vlCDR3) from any anti-CD3 antigen binding domain sequence depicted in FIGS. 2 and 6 of US Publication No. 2014/0288275;
2) the variable heavy and variable light chains from any anti-CD3 antigen binding domain sequence depicted in FIGS. 2 and 6 of US Publication No. 2014/0288275;
3) the scFv domains from any anti-CD3 scFV sequence depicted in FIG. 2 of US Publication No. 2014/0288275;
4) other anti-CD3 variable heavy and variable light chains as are known in the art, that can be combined to form scFvs (or Fabs, when the format is reversed or an alternative format is used); and
5) any of the anti-CD3 antigen binding domains of FIGS. 2, 3, 4, 5, 6, and 7 of U.S. Ser. No. 14/952,714.

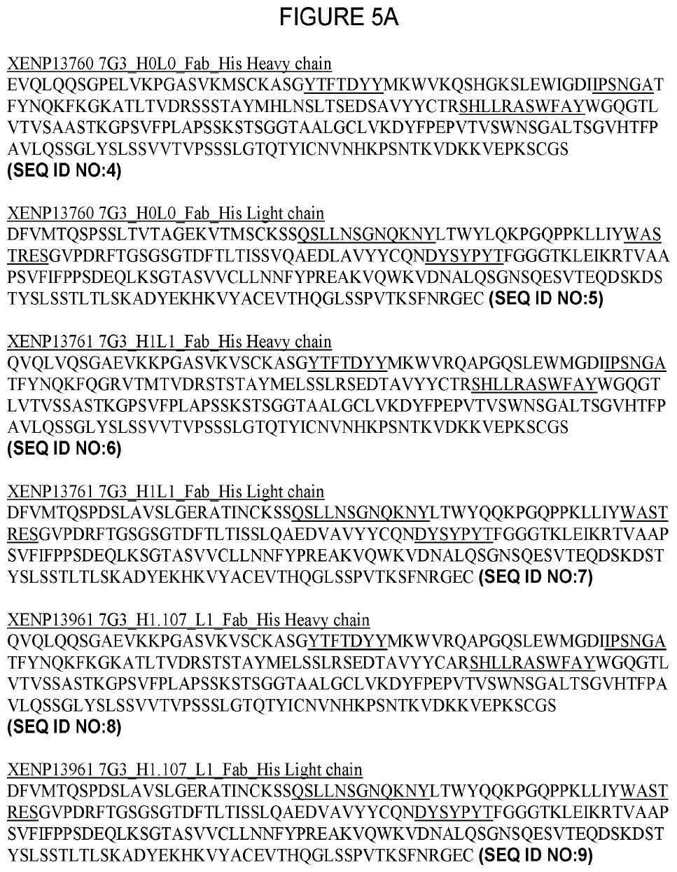

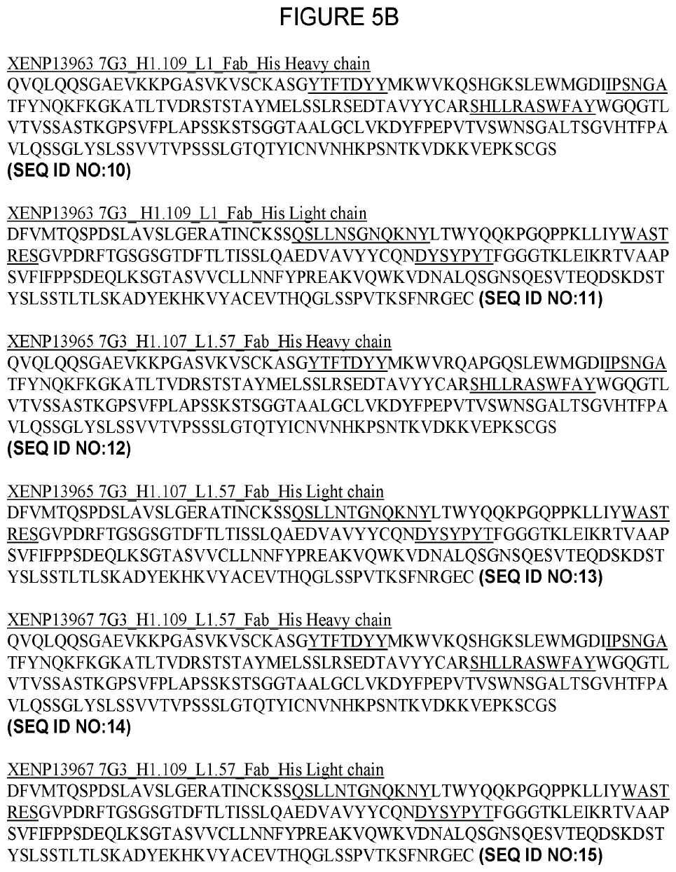

The anti-CD123 Fab binding domain can have the sequence depicted in FIG. 2 or 5, or can be selected from:
1) The set of 6 CDRs (vhCDR1, vhCDR2, vhCDR3, vlCDR1, vlCDR2 and vlCDR3) from any anti-CD123 antigen binding domain sequence depicted in U.S. Ser. No. 62/085,027, including those depicted in FIGS. 2, 3 and 12;
2) The variable heavy and variable light chains from any anti-CD123 antigen binding domain sequence depicted in U.S. Ser. No. 62/085,027, including those depicted in FIGS. 2, 3 and 12; and
3) Other anti-CD123 variable heavy and variable light chains as are known in the art, that can be combined to form Fabs (or scFvs, when the format is reversed or an alternative format is used).

One bispecific antibody of particular use in the present invention, XmAb14045, is shown in FIG. 2 and Table 1 below. XmAb14045 was alternatively known as XENP14045.

TABLE 1
XmAb14045 Anti-CD123 x Anti-CD3 Sequences

| | SEQ ID NO | Sequence |
|---|---|---|
| XmAb14045 Anti- | 1 | QVQLQQSGAEVKKPGASVKVSCKASGYTFTDYYMKWVK |
| CD123 x Anti-CD3 Fab- | | QSHGKSLEWMGDIIPSNGATFYNQKFKGKATLTVDRST |
| scFv-Fc Heavy Chain 1 | | STAYMELSSLRSEDTAVYYCARSHLLRASWFAYWGQGT |
| (Anti-CD123 Fab-Fc | | LVTVSSASTKGPSVFPLAPSSKSTSGGTAALGCLVKDY |
| (7G3_H1.109)) | | FPEPVTVSWNSGALTSGVHTFPAVLQSSGLYSLSSVVT |
| | | VPSSSLGTQTYICNVNHKPSDTKVDKKV/EPKSCDKTH |
| | | TCPPCPAPPVAGPSVFLFPPKPKDTLMISRTPEVTCVV |
| | | VDVKHEDPEVKFNWYVDGVEVHNAKTKPREEEYNSTYR |
| | | VVSVLTVLHQDWLNGKEYKCKVSNKALPAPIEKTISKA |
| | | KGQPREPQVYTLPPSREEMTKNQVSLTCDVSGFYPSDI |
| | | AVEWESDGQPENNYKTTPPVLDSDGSFFLYSKLTVDKS |
| | | RWEQGDVFSCSVMHEALHNHYTQKSLSLSPGK |
| XmAb14045 Anti- | 2 | EVQLVESGGGLVQPGGSLRLSCAASGFTFSTYAMNWVR |
| CD123 x Anti-CD3 Fab- | | QAPGKGLEWVGRIRSKYNNYATYYADSVKGRFTISRDD |
| scFv-Fc Heavy Chain 2 | | SKNTLYLQMNSLRAEDTAVYYCVRHGNFGDSYVSWFAY |
| (Anti-CD3 scFv-Fc | | WGQGTLVTVSSGKPGSGKPGSGKPGSGKPGSQAVVTQE |
| (αCD3_H1.30_L1.47)) | | PSLTVSPGGTVTLTCGSSTGAVTTSNYANWVQQKPGKS |
| | | PRGLIGGTNKRAPGVPARFSGSLLGGKAALTISGAQPE |
| | | DEADYYCALWYSNHWVFGGGTKLTVL/EPKSSDKTHTC |
| | | PPCPAPPVAGPSVFLFPPKPKDTLMISRTPEVTCVVVD |
| | | VKHEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSREQMTKNQVKLTCLVKGFYPSDIAV |
| | | EWESNGQPENNYKTTPPVLDSDGSFFLYSKLTVDKSRW |
| | | QQGNVFSCSVMHEALHNHYTQKSLSLSPGK |
| XmAb14045 Anti- | 3 | DFVMTQSPDSLAVSLGERATINCKSSQSLLNTGNQKNY |
| CD123 x Anti-CD3 Fab- | | LTWYQQKPGQPPKLLIYWASTRESGVPDRFTGSGSGTD |
| scFv-Fc Light Chain | | FTLTISSLQAEDVAVYYCQNDYSYPYTFGGGTKLEIK/ |
| (Anti-CD123 LC | | RTVAAPSVFIFPPSDEQLKSGTASVVCLLNNFYPREAK |
| (7G3_L1.57)) | | VQWKVDNALQSGNSQESVTEQDSKDSTYSLSSTLTLSK |
| | | ADYEKHKVYACEVTHQGLSSPVTKSFNRGEC |
| XmAb14045 Anti- | 19 | QVQLQQSGAEVKKPGASVKVSCKASGYTFTDYYMKWVK |
| CD123 VH | | QSHGKSLEWMGDIIPSNGATFYNQKFKGKATLTVDRST |
| (7G3_H1.109) | | STAYMELSSLRSEDTAVYYCARSHLLRASWFAYWGQGT |
| | | LVTVSSASTKGPSVFPLAPSSKSTSGGTAALGCLVKDY |
| | | FPEPVTVSWNSGALTSGVHTFPAVLQSSGLYSLSSVVT |
| | | VPSSSLGTQTYICNVNHKPSDTKVDKKV |
| XmAb14045 Anti- | 20 | DFVMTQSPDSLAVSLGERATINCKSSQSLLNTGNQKNY |
| CD123 VL (7G3_L1.57) | | LTWYQQKPGQPPKLLIYWASTRESGVPDRFTGSGSGTD |
| | | FTLTISSLQAEDVAVYYCQNDYSYPYTFGGGTKLEIK |
| XmAb14045 Anti-CD3 | 21 | EVQLVESGGGLVQPGGSLRLSCAASGFTFSTYAMNWVR |
| VH (H1.30) | | QAPGKGLEWVGRIRSKYNNYATYYADSVKGRFTISRDD |
| | | SKNTLYLQMNSLRAEDTAVYYCVRHGNFGDSYVSWFAY |
| | | WGQGTLVTVSS |
| XmAb14045 Anti-CD3 | 22 | QAVVTQEPSLTVSPGGTVTLTCGSSTGAVTTSNYANWV |
| VL (L1.47) | | QQKPGKSPRGLIGGTNKRAPGVPARFSGSLLGGKAALT |
| | | ISGAQPEDEADYYCALWYSNHWVFGGGTKLTVL |
| XmAb14045 anti-CD3 | 23 | TYAMN |
| VH CDR1 | | |
| XmAb14045 anti-CD3 | 24 | RIRSKYNNYATYYADSVKG |
| VH CDR2 | | |
| XmAb14045 anti-CD3 | 25 | HGNFGDSYVSWFAY |
| VH CDR3 | | |
| XmAb14045 anti-CD3 | 26 | GSSTGAVTTSNYAN |
| VL CDR1 | | |
| XmAb14045 anti-CD3 | 27 | GTNKRAP |
| VL CDR2 | | |
| XmAb14045 anti-CD3 | 28 | ALWYSNHWV |
| VL CDR3 | | |
| XmAb14045 anti-CD123 | 29 | YTFTDYY |
| VH CDR1 | | |
| XmAb14045 anti-CD123 | 30 | IPSNGA |
| VH CDR2 | | |
| XmAb14045 anti-CD123 | 31 | SHLLRASWFAY |
| VH CDR3 | | |
| XmAb14045 anti-CD123 | 32 | QSLLNTGNQKNY |
| VL CDR1 | | |
| XmAb14045 anti-CD123 | 33 | WASTRES |
| VL CDR2 | | |
| XmAb14045 anti-CD123 | 34 | DYSYPYT |
| VL CDR3 | | |

The XmAb14045 bispecific antibody includes a first monomer comprising SEQ ID NO: 1, a second monomer comprising SEQ ID NO: 2, and a light chain comprising SEQ ID NO: 3. In some embodiments, the bispecific anti-CD123×anti-CD3 antibody includes a first monomer comprising SEQ ID NO: 1, a second monomer comprising SEQ ID NO: 2 and a light chain comprising SEQ ID NO: 3, as depicted in Table 1. In some embodiments, the bispecific anti-CD123×anti-CD3 antibody includes an anti-CD123 variable heavy (VH) domain comprising SEQ ID NO:19, an anti-CD123 variable light (VL) domain comprising SEQ ID NO:20, an anti-CD3 variable heavy (VH) domain comprising SEQ ID NO:21, and an anti-CD3 variable light (VL) domain comprising SEQ ID NO: 22, as depicted in Table 1. In certain embodiments, the bispecific anti-CD123×anti-CD3 antibody includes an anti-CD3 binding domain comprising a VH CDR1 of SEQ ID NO: 23, a VH CDR2 of SEQ ID NO: 24, a VH CDR3 of SEQ ID NO: 25, a VL CDR1 of SEQ ID NO: 26, a VL CDR2 of SEQ ID NO: 27, a VL CDR3 of SEQ ID NO: 28; and an anti-CD123 binding domain comprising a VH CDR1 of SEQ ID NO: 29, a VH CDR2 of SEQ ID NO: 30, a VH CDR3 of SEQ ID NO: 31, a VL CDR1 of SEQ ID NO: 32, a VL CDR2 of SEQ ID NO: 33, and a VL CDR3 of SEQ ID NO: 34, as depicted in Table 1.

The bispecific anti-CD123×anti-CD3 antibodies of the invention are made as is known in the art. The invention further provides nucleic acid compositions encoding the bispecific anti-CD123×anti-CD3 antibodies of the invention. As will be appreciated by those in the art, the nucleic acid compositions will depend on the format and scaffold of the bispecific anti-CD123×anti-CD3 antibodies. Thus, for example, when the format requires three amino acid sequences, such as for the triple F format (e.g. a first amino acid monomer comprising an Fc domain and a scFv, a second amino acid monomer comprising a heavy chain and a light chain), three nucleic acid sequences can be incorporated into one or more expression vectors for expression. Similarly, some formats (e.g. dual scFv formats such as disclosed in FIG. 7) only two nucleic acids are needed; again, they can be put into one or two expression vectors.

As is known in the art, the nucleic acids encoding the components of the invention can be incorporated into expression vectors as is known in the art, and depending on the host cells used to produce the bispecific anti-CD123×anti-CD3 antibodies of the invention. Generally, the nucleic acids are operably linked to any number of regulatory elements (promoters, origin of replication, selectable markers, ribosomal binding sites, inducers, etc.). The expression vectors can be extra-chromosomal or integrating vectors. In some embodiments, the anti-CD123×anti-CD3 antibody is generated from a nucleic acid composition that includes a first nucleic acid that encodes SEQ ID NO: 1, a second nucleic acid that encodes SEQ ID NO: 2, and a third nucleic acid that encodes SEQ ID NO: 3.

The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments. The nucleic acids and/or expression vectors of the invention are then transformed into any number of different types of host cells as is well known in the art, including mammalian, bacterial, yeast, insect and/or fungal cells, with mammalian cells (e.g. CHO cells), finding use in many embodiments. In some embodiments, the anti-CD123×anti-CD3 antibody is generated from an expression vector composition that includes a first expression vector that includes a first nucleic acid that encodes SEQ ID NO: 1, a second expression vector that includes a second nucleic acid that encodes SEQ ID NO: 2, and a third expression vector that includes a third nucleic acid that encodes SEQ ID NO: 3. In some embodiments, the anti-CD123×anti-CD3 antibody is generated from host cell that includes a first expression vector that includes a first nucleic acid that encodes SEQ ID NO: 1, a second expression vector that includes a second nucleic acid that encodes SEQ ID NO: 2, and a third nucleic acid that includes a third nucleic acid that encodes SEQ ID NO: 3.

In some embodiments, nucleic acids encoding each monomer and the optional nucleic acid encoding a light chain, as applicable depending on the format, are each contained within a single expression vector, generally under different or the same promoter controls. In embodiments of particular use in the present invention, each of these two or three nucleic acids are contained on a different expression vector.

The heterodimeric bispecific anti-CD123×anti-CD3 antibodies of the invention are made by culturing host cells comprising the expression vector(s) as is well known in the art. Once produced, traditional antibody purification steps are done, including an ion exchange chromatography step. As discussed in U.S. Ser. No. 14/205,248 and WO2014/145806, hereby incorporated by reference in their entirety and particularly for the discussions concerning purification, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “triple F” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

Once made, the bispecific anti-CD123×anti-CD3 antibodies are administered to human subjects in dosages as outlined herein.

### IV. Pharmaceutical Compositions and Pharmaceutical Administration

The bispecific anti-CD123×anti-CD3 antibodies (e.g., XmAb14045) of the invention can be incorporated into pharmaceutical compositions suitable for administration to a subject for the methods described herein, e.g., weekly, intravenous dosing. Typically, the pharmaceutical composition comprises a bispecific anti-CD123×anti-CD3 antibody of the invention (e.g., XmAb14045) and a pharmaceutically acceptable carrier. As used herein, “pharmaceutically acceptable carrier” includes any and all solvents, dispersion media, coatings, isotonic and absorption delaying agents, and the like that are physiologically compatible and are suitable for administration to a subject for the methods described herein. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as surfactants (such as nonionic surfactants) wetting or emulsifying agents, preservatives or buffers (such as an organic acid, which as a citrate), which enhance the shelf life or effectiveness of the bispecific anti-CD123×anti-CD3 antibody (e.g., XmAb14045). An example of pharmaceutically acceptable carriers include polysorbates (polysorbate-80). In an exemplary embodiment, the pharmaceutical composition comprises an antibody described herein, and a citrate. In an exemplary embodiment, the pharmaceutical composition comprises an antibody described herein, and a polysorbate. In an exemplary embodiment, the pharmaceutical composition comprises an antibody described herein, and a citrate and a polysorbate. In an exemplary embodiment, the pharmaceutical composition comprises an antibody described herein, and sodium citrate. In an exemplary embodiment, the pharmaceutical composition comprises an antibody described herein, and polysorbate-80. In an exemplary embodiment, the pharmaceutical composition comprises an antibody described herein, and sodium citrate and polysorbate-80. In an exemplary embodiment, the pharmaceutical composition comprises an antibody described herein, and sodium chloride. In an exemplary embodiment, the pharmaceutical composition comprises an antibody described herein, and sodium chloride and polysorbate-80. In an exemplary embodiment, the pharmaceutical composition comprises an antibody described herein, and sodium citrate and sodium chloride. In an exemplary embodiment, the pharmaceutical composition comprises an antibody described herein, and sodium citrate, sodium chloride, and polysorbate-80.

The pharmaceutical compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application. Exemplary compositions are in the form of injectable or infusible solutions, such as compositions similar to those used for passive immunization of humans with other antibodies. In an exemplary embodiment, the mode of administration is intravenous. In an exemplary embodiment, the antibody is administered by intravenous infusion or injection.

Pharmaceutical compositions typically must be sterile and stable under the conditions of manufacture and storage. The pharmaceutical composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated herein, as required, followed by filtered sterilization. Generally, dispersions are prepared by incorporating the antibody into a sterile vehicle that contains a basic dispersion medium and the required other ingredients from those enumerated herein. In the case of sterile powders for the preparation of sterile injectable solutions, in an exemplary embodiment, the method of preparation is vacuum drying and freeze-drying that yields a powder of the antibody plus any additional desired carrier from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The bispecific anti-CD123×anti-CD3 antibodies of the present invention can be administered by a variety of methods known in the art. In an exemplary embodiment, the route/mode of administration is intravenous injection. As will be appreciated by the skilled artisan, the route and/or mode of administration will vary depending upon the desired results. In certain embodiments, the bispecific anti-CD123×anti-CD3 antibody (e.g., XmAb14045) may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyethylene glycol (PEG), polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

### V. Methods of Treating Leukemia

Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive build up of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

In an exemplary embodiment, the leukemia is selected from the group consisting of acute lymphocytic leukemia (ALL), acute myeloid leukemia (AML), chronic myeloid leukemia (CML), and hairy cell leukemia (HCL). In an exemplary embodiment, the leukemia is acute lymphocytic leukemia (ALL). In an exemplary embodiment, the leukemia is acute myeloid leukemia (AML). In an exemplary embodiment, the leukemia is chronic myeloid leukemia (CML). In an exemplary embodiment, the leukemia is chronic phase chronic myeloid leukemia. In an exemplary embodiment, the leukemia is accelerated phase chronic myeloid leukemia. In an exemplary embodiment, the leukemia is blast phase chronic myeloid leukemia. In an exemplary embodiment, the leukemia is hairy cell leukemia (HCL). In an exemplary embodiment, the leukemia is classic hairy cell leukemia (HCLc). In an exemplary embodiment, the leukemia is variant hairy cell leukemia (HCLv). In an exemplary embodiment, the leukemia is acute myeloid leukemia (AML), and the acute myeloid leukemia is primary acute myeloid leukemia. In an exemplary embodiment, the leukemia is acute myeloid leukemia (AML), and the acute myeloid leukemia is secondary acute myeloid leukemia. In an exemplary embodiment, the leukemia is erythroleukemia. In an exemplary embodiment, the leukemia is eosinophilic leukemia. In an exemplary embodiment, the leukemia is acute myeloid leukemia (AML), and the acute myeloid leukemia does not include acute promyelocytic leukemia. In an exemplary embodiment, the leukemia is acute myeloid leukemia (AML), and the acute myeloid leukemia is blastic plasmacytoid dendritic cell neoplasm. In an exemplary embodiment, the leukemia is B-cell acute lymphocytic leukemia (B-ALL). In an exemplary embodiment, the leukemia is T-cell acute lymphocytic leukemia (T-ALL).

### V. Subject Selection

Subjects can be selected based on CD123 expression level in a sample (e.g., a tissue sample or a blood sample) obtained from the subject. CD123 expression level can be determined by an assay known in the art, e.g., flow cytometry, immunohistochemistry, Western blotting, immunofluorescent assay, radioimmunoassay (RIA), enzyme-linked immunosorbent assay (ELISA), homogeneous time resolved fluorescence (HTRF), positron emission tomography (PET), or any other immune detection with an antibody or antibody fragment against CD123 protein.

Blood samples can be collected from a subject using any method known in the art, e.g., by venipuncture or fingerstick. Particular types of blood cells can be isolated, expanded, frozen, and used at a later time. Tissue samples can be obtained from a subject using any method known in the art, e.g., by biopsy or surgery. CT imaging, ultrasound, or an endoscope can be used to guide this type of procedure. The sample may be flash frozen and stored at −80° C. for later use. The sample may also be fixed with a fixative, such as formaldehyde, paraformaldehyde, or acetic acid/ethanol. RNA or protein may be extracted from a fresh, frozen or fixed sample for analysis.

### VI. Dosage Regimen

In some embodiments, the bispecific anti-CD123×anti-CD3 antibody (e.g., XmAb14045) is administered according to a dosage regimen described herein. Dosage regimens are adjusted to provide the optimum desired response (e.g., a therapeutic response). The efficient dosages and the dosage regimens for the bispecific anti-CD123×anti-CD3 antibodies used in the present invention depend on the disease or condition to be treated and may be determined by the persons skilled in the art.

In an exemplary embodiment, the bispecific anti-CD123×anti-CD3 antibody (e.g., XmAb14045) is administered intravenously by infusion once every 6-8 days in an amount of from about 1 ng/kg to about 800 ng/kg.

In an exemplary embodiment, the bispecific anti-CD123×anti-CD3 antibody (e.g., XmAb14045) is administered intravenously by infusion monthly in an amount of from about 30 ng/kg to about 750 ng/kg, e.g., about 75 ng/kg to about 750 ng/kg, about 75 ng/kg to about 700 ng/kg, about 75 ng/kg to about 650 ng/kg, about 75 ng/kg to about 600 ng/kg, about 75 ng/kg to about 550 ng/kg, about 75 ng/kg to about 500 ng/kg, about 75 ng/kg to about 450 ng/kg, about 75 ng/kg to about 400 ng/kg, about 75 ng/kg to about 350 ng/kg, about 75 ng/kg to about 300 ng/kg, about 75 ng/kg to about 250 ng/kg, about 75 ng/kg to about 200 ng/kg, about 75 ng/kg to about 150 ng/kg, or about 75 ng/kg to about 100 ng/kg.

In an exemplary embodiment, the bispecific anti-CD123×anti-CD3 antibody (e.g., XmAb14045) is administered intravenously by infusion every other week in an amount of from about 30 ng/kg to about 750 ng/kg, e.g., about 75 ng/kg to about 750 ng/kg, about 75 ng/kg to about 700 ng/kg, about 75 ng/kg to about 650 ng/kg, about 75 ng/kg to about 600 ng/kg, about 75 ng/kg to about 550 ng/kg, about 75 ng/kg to about 500 ng/kg, about 75 ng/kg to about 450 ng/kg, about 75 ng/kg to about 400 ng/kg, about 75 ng/kg to about 350 ng/kg, about 75 ng/kg to about 300 ng/kg, about 75 ng/kg to about 250 ng/kg, about 75 ng/kg to about 200 ng/kg, or about 75 ng/kg to about 150 ng/kg, or about 75 ng/kg to about 100 ng/kg.

In an exemplary embodiment, the bispecific anti-CD123×anti-CD3 antibody (e.g., XmAb14045) is administered by infusion for a period of between about one hour and about three hours. In an exemplary embodiment, the bispecific anti-CD123×anti-CD3 antibody (e.g., XmAb14045) is administered by infusion for a period of about two hours. In an exemplary embodiment, the bispecific anti-CD123×anti-CD3 antibody (e.g., XmAb14045) is administered by infusion for a period of two hours.

In an exemplary embodiment, the bispecific anti-CD123×anti-CD3 antibody (e.g., XmAb14045) is administered once every 6-8 days for between about 1 and about 9 weeks. In an exemplary embodiment, the bispecific anti-CD123×anti-CD3 antibody (e.g., XmAb14045) is administered once every 6-8 days for between about 2 and about 7 weeks. In an exemplary embodiment, the bispecific anti-CD123×anti-CD3 antibody (e.g., XmAb14045) is administered once every 6-8 days for between about 3 and about 9 weeks. In an exemplary embodiment, the bispecific anti-CD123×anti-CD3 antibody (e.g., XmAb14045) is administered once every 6-8 days for between about 1 and about 8 weeks. In an exemplary embodiment, the bispecific anti-CD123×anti-CD3 antibody (e.g., XmAb14045) is administered once every 6-8 days for between about 3 and about 5 weeks. In an exemplary embodiment, the bispecific anti-CD123×anti-CD3 antibody (e.g., XmAb14045) is administered once every 6-8 days for about 4 weeks. In an exemplary embodiment, the bispecific anti-CD123×anti-CD3 antibody (e.g., XmAb14045) is administered once every 6-8 days for 4 weeks. In an exemplary embodiment, the bispecific anti-CD123×anti-CD3 antibody (e.g., XmAb14045) is administered once every 6-8 days for between about 7 and about 9 weeks. In an exemplary embodiment, the bispecific anti-CD123×anti-CD3 antibody (e.g., XmAb14045) is administered once every 6-8 days for about 8 weeks. In an exemplary embodiment, the bispecific anti-CD123×anti-CD3 antibody (e.g., XmAb14045) is administered once every 6-8 days for 8 weeks.

The dosage may be determined or adjusted by measuring the amount of bispecific anti-CD123×anti-CD3 antibody (e.g., XmAb14045) of the present invention in the blood upon administration using techniques known in the art, for instance taking out a biological sample and using anti-idiotypic antibodies which target the antigen binding region of the bispecific anti-CD123×anti-CD3 antibody (e.g., XmAb14045).

In an exemplary embodiment, the amount is between about 3 ng/kg and about 750 ng/kg.

In an exemplary embodiment, the amount is between about 30 ng/kg and about 750 ng/kg. In an exemplary embodiment, the amount is between about 75 ng/kg and about 750 ng/kg.

In an exemplary embodiment, the amount is between about 1 ng/kg and about 5 ng/kg. In an exemplary embodiment, the amount is between about 2 ng/kg and about 4 ng/kg. In an exemplary embodiment, the amount is about 3 ng/kg. In an exemplary embodiment, the amount is 3 ng/kg.

In an exemplary embodiment, the amount is between about 1 ng/kg and about 20 ng/kg. In an exemplary embodiment, the amount is between about 5 ng/kg and about 15 ng/kg. In an exemplary embodiment, the amount is between about 7 ng/kg and about 13 ng/kg. In an exemplary embodiment, the amount is between about 9 ng/kg and about 11 ng/kg. In an exemplary embodiment, the amount is about 10 ng/kg. In an exemplary embodiment, the amount is 10 ng/kg.

In an exemplary embodiment, the amount is between about 10 ng/kg and about 50 ng/kg. In an exemplary embodiment, the amount is between about 20 ng/kg and about 40 ng/kg. In an exemplary embodiment, the amount is between about 25 ng/kg and about 35 ng/kg. In an exemplary embodiment, the amount is about 30 ng/kg. In an exemplary embodiment, the amount is 30 ng/kg.

In an exemplary embodiment, the amount is between about 25 ng/kg and about 150 ng/kg. In an exemplary embodiment, the amount is between about 50 ng/kg and about 125 ng/kg. In an exemplary embodiment, the amount is between about 50 ng/kg and about 100 ng/kg. In an exemplary embodiment, the amount is between about 55 ng/kg and about 95 ng/kg. In an exemplary embodiment, the amount is between about 60 ng/kg and about 90 ng/kg. In an exemplary embodiment, the amount is between about 65 ng/kg and about 85 ng/kg. In an exemplary embodiment, the amount is between about 70 ng/kg and about 80 ng/kg. In an exemplary embodiment, the amount is about 75 ng/kg. In an exemplary embodiment, the amount is 75 ng/kg.

In an exemplary embodiment, the amount is between about 50 ng/kg and about 250 ng/kg. In an exemplary embodiment, the amount is between about 75 ng/kg and about 225 ng/kg. In an exemplary embodiment, the amount is between about 100 ng/kg and about 200 ng/kg. In an exemplary embodiment, the amount is between about 125 ng/kg and about 175 ng/kg. In an exemplary embodiment, the amount is about 150 ng/kg. In an exemplary embodiment, the amount is 150 ng/kg.

In an exemplary embodiment, the amount is between about 100 ng/kg and about 500 ng/kg. In an exemplary embodiment, the amount is between about 200 ng/kg and about 400 ng/kg. In an exemplary embodiment, the amount is between about 200 ng/kg and about 400 ng/kg. In an exemplary embodiment, the amount is between about 225 ng/kg and about 375 ng/kg. In an exemplary embodiment, the amount is between about 250 ng/kg and about 350 ng/kg. In an exemplary embodiment, the amount is between about 275 ng/kg and about 325 ng/kg. In an exemplary embodiment, the amount is about 300 ng/kg. In an exemplary embodiment, the amount is 300 ng/kg.

In an exemplary embodiment, the amount is between about 350 ng/kg and about 650 ng/kg. In an exemplary embodiment, the amount is between about 400 ng/kg and about 600 ng/kg. In an exemplary embodiment, the amount is between about 450 ng/kg and about 550 ng/kg. In an exemplary embodiment, the amount is between about 475 ng/kg and about 525 ng/kg. In an exemplary embodiment, the amount is about 500 ng/kg. In an exemplary embodiment, the amount is 500 ng/kg.

In an exemplary embodiment, the amount is between about 600 ng/kg and about 900 ng/kg. In an exemplary embodiment, the amount is between about 650 ng/kg and about 850 ng/kg. In an exemplary embodiment, the amount is between about 700 ng/kg and about 800 ng/kg. In an exemplary embodiment, the amount is between about 725 ng/kg and about 775 ng/kg. In an exemplary embodiment, the amount is about 750 ng/kg. In an exemplary embodiment, the amount is 750 ng/kg.

In some embodiments, the bispecific anti-CD123×anti-CD3 antibody (e.g., XmAb14045) is administered intravenously. In some embodiments, the bispecific anti-CD123×anti-CD3 antibody (e.g., XmAb14045) is administered weekly until disease progression, unacceptable toxicity, or individual choice.

In some embodiments, the bispecific anti-CD123×anti-CD3 antibody (e.g., XmAb14045) is a front line therapy, second line therapy, third line therapy, fourth line therapy, fifth line therapy, or sixth line therapy.

In some embodiments, the bispecific anti-CD123×anti-CD3 antibody (e.g., XmAb14045) treats a refractory leukemia. In some embodiments, the bispecific anti-CD123×anti-CD3 antibody (e.g., XmAb14045) is a maintenance therapy.

A medical professional having ordinary skill in the art may readily determine and prescribe the effective amount of the antibody composition required. For example, a physician could start doses of the medicament employed in the antibody composition at levels lower than that required in order to achieve the desired therapeutic effect and gradually increase the dosage until the desired effect is achieved.

### VII. Treatment Modalities

In the methods of the invention, treatment is used to provide a positive therapeutic response with respect to a leukemia. By “positive therapeutic response” is intended an improvement in the leukemia, and/or an improvement in the symptoms associated with the leukemia. For example, a positive therapeutic response would refer to one or more of the following improvements in the leukemia: (1) a reduction in the number of CD123⁺ leukemia-associated cells, including CD123⁺ peripheral blood basophils and/or marrow basophils; (2) an increase in CD123⁺ leukemia-associated cell death; (3) inhibition of CD123⁺ leukemia-associated cell survival; (5) inhibition (i.e., slowing to some extent, preferably halting) of CD123⁺ cell proliferation; (6) an increased human subject survival rate; and (7) some relief from one or more symptoms associated with the leukemia.

Positive therapeutic responses in any given leukemia can be determined by standardized response criteria specific to that leukemia.

In addition to these positive therapeutic responses, the subject undergoing treatment may experience the beneficial effect of an improvement in the symptoms associated with the leukemia. In an exemplary embodiment, a treatment of leukemia is selected from the group consisting of feeling less tired, feeling less weak, feeling less dizzy or lightheaded, reduction in shortness of breath, reduction in fever, quicker response to infections, reduction in ease of bruising, reduction in bleeding episodes, weight gain, reduction in night sweats, gain of appetite, reduction in abdominal swelling, reduction in lymph node swelling, reduction in bone or joint pain, and reduction in thymus swelling.

An improvement in the leukemia may be characterized as a complete response. By “complete response” is intended an absence of clinically detectable disease with normalization of any previously abnormal radiographic studies, bone marrow, and cerebrospinal fluid (CSF) or abnormal monoclonal protein in the case of myeloma.

Such a response may persist for at least 4 to 8 weeks, or sometimes 6 to 8 weeks, following treatment according to the methods of the invention. Alternatively, an improvement in the leukemia may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody are outweighed by the therapeutically beneficial effects.

A “therapeutically effective amount” for therapy may also be measured by its ability to stabilize the progression of the leukemia. The ability of an antibody to inhibit leukemia may be evaluated in an animal model system predictive of efficacy in a human.

Alternatively, this property of an antibody composition may be evaluated by examining the ability of the antibody to inhibit cell growth or to induce apoptosis by in vitro assays known to the skilled practitioner. A therapeutically effective amount of a bispecific anti-CD123×anti-CD3 antibody (e.g., XmAb14045) reduce the number of CD123⁺ leukemia-associated cells, or improve other aspects related to the leukemia (such as those described herein), and/or otherwise ameliorate symptoms in a human subject (such as those also described herein). One of ordinary skill in the art would be able to determine such amounts based on such factors as the subject's size, the severity of the subject's symptoms, and the particular antibody composition or route of administration selected.

### VIII. Combination Therapy

In one aspect, the invention provides a method for treating a CD123-expressing cancer in a subject, comprising administering to the subject having the CD123-expressing cancer an intravenous dose of a bispecific anti-CD123×anti-CD3 antibody, for a time period sufficient to treat the CD123-expressing cancer, in combination with at least one other therapeutic agent. In an exemplary embodiment, the at least one other therapeutic agent is an anti-cancer agent or a side-effect ameliorating agent. In an exemplary embodiment, the at least one other therapeutic agent is radiation, a chemotherapeutic agent, an antibody, or a side-effect ameliorating agent.

In certain instances, a bispecific anti-CD123×anti-CD3 antibody (e.g., XmAb14045) described herein can be used in combination with at least one other therapeutic agent. Administered “in combination”, as used herein, means that two (or more) different therapeutic agents are administered to the subject during the course of the subject's affliction with the disorder, e.g., the two or more therapeutic agents are administered after the subject has been diagnosed with the disorder and before the disorder has been cured or eliminated or treatment has ceased for other reasons. In some embodiments, the administration of one therapeutic agent is still occurring when the administration of the second begins, so that there is overlap in terms of administration. This is sometimes referred to herein as “simultaneous” or “concurrent administration”. In other embodiments, the administration of one therapeutic agent ends before the administration of the other therapeutic agent begins. In some embodiments of either case, the treatment is more effective because of combined administration. For example, the second therapeutic agent is more effective, e.g., an equivalent effect is seen with less of the second agent, or the second agent reduces symptoms to a greater extent, than would be seen if the second agent were administered in the absence of the first agent, or the analogous situation is seen with the first agent. In some embodiments, administration is such that the reduction in a symptom, or other parameter related to the disorder is greater than what would be observed with one therapeutic agent administered in the absence of the other. The effect of the therapeutic agents on the subject can be partially additive, wholly additive, or greater than additive. The administration can be such that an effect of the first treatment administration is still detectable when the second is administered.

The bispecific anti-CD123×anti-CD3 antibody (e.g., XmAb14045) described herein and the at least one other therapeutic agent can be administered simultaneously, in the same or in separate compositions, or sequentially. For sequential administration, the bispecific anti-CD123×anti-CD3 antibody (e.g., XmAb14045) described herein can be administered first, and the at least one other therapeutic agent can be administered second, or the order of administration can be reversed.

The bispecific anti-CD123×anti-CD3 antibody (e.g., XmAb14045) and/or other therapeutic agents, procedures or modalities can be administered during periods of active disorder, or during a period where there is persistent MRD, or during a period of remission or less active disease. The bispecific anti-CD123×anti-CD3 antibody (e.g., XmAb14045) can be administered before the other treatment, concurrently with the treatment, post-treatment, or during remission of the disorder.

When administered in combination, the bispecific anti-CD123×anti-CD3 antibody (e.g., XmAb14045) and the additional therapeutic agent (e.g., second or third therapeutic agent), or all, can be administered in an amount or dose that is higher, lower or the same than the amount or dosage of each therapeutic agent used individually, e.g., as a monotherapy. In some embodiments, the administered amount or dosage of the bispecific anti-CD123×anti-CD3 antibody (e.g., XmAb14045), the additional therapeutic agent (e.g., second or third therapeutic agent), or all, is lower (e.g., at least 20%, at least 30%, at least 40%, or at least 50%) than the amount or dosage of each therapeutic agent used individually, e.g., as a monotherapy. In other embodiments, the amount or dosage of the bispecific anti-CD123×anti-CD3 antibody (e.g., XmAb14045), the additional therapeutic agent (e.g., second or third therapeutic agent), or all, that results in a desired effect (e.g., treatment of cancer) is lower (e.g., at least 20%, at least 30%, at least 40%, or at least 50% lower) than the amount or dosage of each therapeutic agent used individually, e.g., as a monotherapy, required to achieve the same therapeutic effect.

In further aspects, a bispecific anti-CD123×anti-CD3 antibody (e.g., XmAb14045) described herein may be administered with in combination with at least one therapeutic agent which is an anti-cancer agent and/or a side effect ameliorating agent.

### VIII. A) Anti-Cancer Agent

In an exemplary embodiment, a bispecific anti-CD123×anti-CD3 antibody (e.g., XmAb14045) described herein may be administered with in combination with at least one therapeutic agent which is an anti-cancer agent. In an exemplary embodiment, the anti-cancer agent is a chemotherapeutic, radiation, or antibody (for example antibodies directed against checkpoint inhibitors). In an exemplary embodiment, the anti-cancer agent is an immunoablative agent such as alemtuzumab, other antibody therapies, cytoxan, fludarabine, rapamycin, mycophenolic acid, steroids, FR90165, cytokines, irradiation, or peptide vaccine, such as that described in Izumoto et al. 2008 J Neurosurg 108:963-971. In an exemplary embodiment, the anti-cancer agent is an immunosuppressive agent. In an exemplary embodiment, the immunosuppressive agent is cyclosporin, azathioprine, methotrexate, mycophenolate, or FK506.

### VIII. a1) Radiation

In one embodiment, a bispecific anti-CD123×anti-CD3 antibody (e.g., XmAb14045) described herein can be used in combination with radiation.

### VIII. a2) Chemotherapeutics

In one embodiment, a bispecific anti-CD123×anti-CD3 antibody (e.g., XmAb14045) described herein can be used in combination with an anti-cancer agent.

In an exemplary embodiment, the anti-cancer agent is a chemotherapeutic. In an exemplary embodiment, the chemotherapeutic is selected from the group consisting of alkylating agent, anti-metabolite, kinase inhibitor, proteasome inhibitor, vinca alkaloid, anthracycline, antitumor antibiotic, aromatase inhibitor, topoisomerase inhibitor, mTOR inhibitor, and retinoid.

### VIII. a2A) Alkylating Agents

In an exemplary embodiment, the anti-cancer agent is a chemotherapeutic, which is an alkylating agent. In an exemplary embodiment, the alkylating agent is a nitrogen mustard, nitrosourea, alkyl sulfonate, triazine, aziridine, platinum complex, or non-classical alkylating agent.

In an exemplary embodiment, the alkylating agent is a nitrogen mustard. In an exemplary embodiment, the alkylating agent is a nitrogen mustard, which is mechlorethamine (mechlorethamine HCl), ifosfamide (IFEX®), melphalan (Alkeran®), chlorambucil, cyclophosphamide, or a derivative thereof. In an exemplary embodiment, the alkylating agent is a nitrogen mustard, which is trofosfamide, estramustine, or a derivative thereof.

In an exemplary embodiment, the alkylating agent is a nitrosourea. In an exemplary embodiment, the alkylating agent is a nitrosourea, which is N-Nitroso-N-methylurea (MNU), streptozocin, carmustine (BCNU), lomustine (CCNU), bendamustine (such as bendamustine HCl), or a derivative thereof. In an exemplary embodiment, the alkylating agent is a nitrosourea, which is semustine, fotemustine, nimustine, ranimustine, or a derivative thereof.

In an exemplary embodiment, the alkylating agent is an alkyl sulfonate. In an exemplary embodiment, the alkylating agent is an alkyl sulfonate, which is busulfan, or a derivative thereof. In an exemplary embodiment, the alkylating agent is an alkyl sulfonate, which is treosulfan, mannosulfan, or a derivative thereof.

In an exemplary embodiment, the alkylating agent is a triazine. In an exemplary embodiment, the alkylating agent is a triazine, which is dacarbazine, mitozolomide, temozolomide (Temodar®), or a derivative thereof.

In an exemplary embodiment, the alkylating agent is an aziridine. In an exemplary embodiment, the alkylating agent is an aziridine, which is thiotepa, altretamine, or a derivative thereof. In an exemplary embodiment, the alkylating agent is an aziridine, which is triaziquone, carboquone, mytomycin, or a derivative thereof.

In an exemplary embodiment, the alkylating agent is a platinum complex. In an exemplary embodiment, the alkylating agent is a platinum complex, which is cisplatin, carboplatin, oxaliplatin, or a derivative thereof.

In an exemplary embodiment, the alkylating agent is a non-classical alkylating agent. In an exemplary embodiment, the non-classical alkylating agent is procarbazine, hexamethylmelamine, or a derivative thereof. In an exemplary embodiment, the alkylating agent is trabectedin, or a derivative thereof

### VIII. a2B) Anti-Metabolites

In an exemplary embodiment, the anti-cancer agent is a chemotherapeutic, which is an anti-metabolite. In an exemplary embodiment, the anti-metabolite is a pyrimidine analog, purine analog, or folate antagonist.

In an exemplary embodiment, the anti-metabolite is a pyrimidine analog. In an exemplary embodiment, the anti-metabolite is a pyrimidine analog which is a fluoropyrimidine. In an exemplary embodiment, the fluoropyrimidine is 5-fluorouracil, capecitabine, carmofur, floxuridine, doxifluridine, tegafur, or a derivative thereof. In an exemplary embodiment, the anti-metabolite is a pyrimidine analog which is cytarabine, gemcitabine, decitabine, azacitidine, or a derivative thereof. In an exemplary embodiment, the anti-metabolite is an adenosine deaminase inhibitor.

In an exemplary embodiment, the anti-metabolite is a purine analog. In an exemplary embodiment, the anti-metabolite is a purine analog, which is fludarabine (also known as 2-fluoro-ara-amp), nelarabine, clofarabine, or a derivative thereof. In an exemplary embodiment, the purine analog is an adenosine analog. In an exemplary embodiment, the adenosine analog is fludarabine (such as fludarabine phosphate), cladribine, pentostatin, or a derivative thereof. In an exemplary embodiment, the purine analog is a guanine analog. In an exemplary embodiment, the guanine analog is thioguanine, 6-mercaptopurine (6-MP), or a derivative thereof.

In an exemplary embodiment, the anti-metabolite is a folate antagonist, which is methotrexate, pemetrexed, or a derivative thereof.

### VIII. a2C) Kinase Inhibitors

In an exemplary embodiment, the anti-cancer agent is a chemotherapeutic, which is a kinase inhibitor. In an exemplary embodiment, the kinase inhibitor is a tyrosine kinase inhibitor. In an exemplary embodiment, the kinase inhibitor is a Src kinase inhibitor. In an exemplary embodiment, the kinase inhibitor is a Bcr-Abl tyrosine kinase inhibitor. In an exemplary embodiment, the kinase inhibitor is asciminib, imatinib (Gleevec®), nilotinib (Tasinga®), ponatinib (Iclusig®), bosutinib (Pfizer), or dasatinib (Sprycel®). In an exemplary embodiment, the kinase inhibitor is a spleen tyrosine kinase (syk) inhibitor. In an exemplary embodiment, the kinase inhibitor is fostamatinib (Tavalisse®)(Rigel). In an exemplary embodiment, the kinase inhibitor is a Bruton's tyrosine kinase (Btk) inhibitor. In an exemplary embodiment, the kinase inhibitor is zanubrutinib also known as BGB-3111 (BeiGene), ibrutinib (e.g., Imbruvica®), evobrutinib (EMD Serono), or acalabrutinib (Acerta/AstraZeneca). In an exemplary embodiment, the kinase inhibitor is a receptor tyrosine kinase (RTK) inhibitor. In an exemplary embodiment, the kinase inhibitor inhibits the tyrosine kinase domain of the epidermal growth factor receptor (EGFR). In an exemplary embodiment, the kinase inhibitor inhibits the tyrosine kinase domain of the epidermal growth factor receptor (EGFR). In an exemplary embodiment, the kinase inhibitor is gefitinib (Iressa®), erlotinib (Tarceva®), pyrotinib, also known as HTI-1001 (Hengrui Therapeutics), afatinib (Gilotrif®), or lapatinib (Tykerb®). In an exemplary embodiment, the kinase inhibitor is a platelet-derived growth factor receptor (PDGF-R) inhibitor. In an exemplary embodiment, the kinase inhibitor is a vascular endothelial growth factor receptor (VEGFR) inhibitor. In an exemplary embodiment, the kinase inhibitor is sunitinib (Sutent®), lenvatinib (Lenvima®), or axitinib, formerly known as AG013736 (Inlyta®). In an exemplary embodiment, the kinase inhibitor is a vascular endothelial growth factor receptor-2 (VEGFR2) inhibitor. In an exemplary embodiment, the kinase inhibitor is apatinib, also known as YN968D1 (Jiangsu Hengrui) vatalanib, cabozantinib (Cabometyx®), golvatinib also known as E7050, or regorafenib (BAY 73-4506, Stivarga®). In an exemplary embodiment, the kinase inhibitor is a Raf kinase inhibitor. In an exemplary embodiment, the kinase inhibitor is sorafenib (Nexavar®). In an exemplary embodiment, the kinase inhibitor is an Axl receptor tyrosine kinase. In an exemplary embodiment, the kinase inhibitor is bemcentinib, also known as BGB324 also known as R428 (Rigel), gilteritinib (Astellas). In an exemplary embodiment, the tyrosine kinase inhibitor is neratinib (HER2 Her1 Her4), toceranib, or a derivative thereof. In an exemplary embodiment, the kinase inhibitor is a phosphatidylinositol-4,5-bisphosphate 3-kinase (PI3K(s)). In an exemplary embodiment, the kinase inhibitor is idelalisib (e.g., Zydelig®) (Gilead) or alpelisib. In an exemplary embodiment, the kinase inhibitor is a Chkl inhibitor. In an exemplary embodiment, the kinase inhibitor is rabusertib also known as LY2603618 (Eli Lilly).

### VIII. a2D) Proteosome Inhibitors

In an exemplary embodiment, the anti-cancer agent is a chemotherapeutic, which is a proteasome inhibitor. In an exemplary embodiment, the proteasome inhibitor is bortezomib (Velcade®), carfilzomib, ixazomid, or a derivative thereof

### VIII. a2E) Vinca Alkaloids

In an exemplary embodiment, the anti-cancer agent is a chemotherapeutic, which is a vinca alkaloid. In an exemplary embodiment, the anti-cancer agent is a chemotherapeutic, which is a monoterpenoid indole alkaloid. In an exemplary embodiment, the anti-cancer agent is a vinca alkaloid, which is vinblastine, vinorelbine, vincristine, vindesine, or a derivative thereof.

### VIII. a2F) Anthracyclines

In an exemplary embodiment, the anti-cancer agent is a chemotherapeutic, which is an anthracycline. In an exemplary embodiment, the anthracycline is daunorubicin, also known as daunomycin, doxorubicin (Adriamycin®) (e.g., liposomal doxorubicin), epirubicin, idarubicin (Idamycin®), valrubicin, or a derivative thereof.

### IVII. A2G) Other Antitumor Antibiotics

In an exemplary embodiment, the anti-cancer agent is a chemotherapeutic, which is an antitumor antibiotic. In an exemplary embodiment, the antitumor antibiotic is actinomycin, bleomycin, dactinomycin, mytomycin, or a derivative thereof. In an exemplary embodiment, the antitumor antibiotic is actinomycin-D or mytomycin-C, or a derivative thereof.

In an exemplary embodiment, the anti-cancer agent is a chemotherapeutic, which is a microtubule agent. In an exemplary embodiment, the microtubule agent is docetaxel, paclitaxel, or a derivative thereof.

### VIII. a2H) Aromatase Inhibitors

In an exemplary embodiment, the anti-cancer agent is a chemotherapeutic, which is an aromatase inhibitor. In an exemplary embodiment, the aromatase inhibitor is a steroidal inhibitor. In an exemplary embodiment, the aromatase steroidal inhibitor is exemestane (Aromasin®), formestane, or a derivative thereof. In an exemplary embodiment, the aromatase inhibitor is a non-steroidal inhibitor. In an exemplary embodiment, the aromatase non-steroidal inhibitor is anastrozole (Arimidex®), letrozole (Femara®), or a derivative thereof.

### VIII. a2I) Topoisomerase Inhibitors

In an exemplary embodiment, the anti-cancer agent is a chemotherapeutic, which is a topoisomerase inhibitor. In an exemplary embodiment, the topoisomerase inhibitor is a topoisomerase I inhibitor. In an exemplary embodiment, the topoisomerase I inhibitor is camptothecin, or a derivative thereof. In an exemplary embodiment, the topoisomerase I inhibitor is irinotecan, topotecan, or a derivative thereof. In an exemplary embodiment, the topoisomerase inhibitor is a topoisomerase II inhibitor. In an exemplary embodiment, the topoisomerase II inhibitor is etoposide, teniposide, mitoxantrone (Novantrone®), or a derivative thereof.

### VIII. a2J) mTOR Inhibitors

In an exemplary embodiment, the anti-cancer agent is a chemotherapeutic, which is an mTOR inhibitor. In an exemplary embodiment, the mTOR inhibitor is rapamycin or a rapalog. In an exemplary embodiment, the mTOR inhibitor is temsirolimus (Torisel®), everolimus (Afinitor®), ridaforolimus, or a derivative thereof. In an exemplary embodiment, the mTOR inhibitor is a dual PI3K/mTOR inhibitor. In an exemplary embodiment, the dual PI3K/mTOR inhibitor is dactolisib, GSK2126458, or a derivative thereof. In an exemplary embodiment, the mTOR inhibitor is ATP-competitive mTORC1/mTORC2 inhibitor. In an exemplary embodiment, the ATP-competitive mTORC1/mTORC2 inhibitor is sapanisertib, or a derivative thereof.

### VIII. a2K) Retinoids

In an exemplary embodiment, the anti-cancer agent is a chemotherapeutic, which is a retinoid. In an exemplary embodiment, the retinoid is all-trans retinoic acid (tretinoin), alitretinoin (9-cis RA), bexarotene (Targretin®), or a derivative thereof.

Exemplary chemotherapeutics include an anthracenedione derivative (e.g., mitoxantrone), an immune cell antibody (e.g., gemtuzumab, gemtuzumab ozogamicin, rituximab, obinutuzumab, ofatumumab, ibritumomab tiuxetan, brentuximab), an anti-CD52 Ab such as alemtuzumab (Campath®). In an exemplary embodiment, the chemotherapeutic agent is tositumomab or aclacinomycin A or gliotoxin or pegaspargase.

General chemotherapeutic agents considered for use in combination therapies include bleomycin sulfate (Blenoxane®), busulfan (Myleran®), capecitabine (Xeloda), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine liposome injection (DepoCyt®), dacarbazine (DTIC-Dome), dactinomycin (Actinomycin D, Cosmegan), daunorubicin HCl(Cerubidine), daunorubicin citrate liposome injection (DaunoXome®), dexamethasone, docetaxel (Taxotere®), doxorubicin HCl(Adriamycin®, Rubex®), etoposide (Vepesid®), fludarabine phosphate (Fludara®), 5-fluorouracil (Adrucil®, Efudex®), gemcitabine (difluorodeoxycitidine), hydroxyurea (Hydrea®), idarubicin (Idamycin®), irinotecan (Camptosar), L-asparaginase (ELSPAR®), leucovorin calcium, 6-mercaptopurine (Purinethol®), methotrexate (Folex®), paclitaxel (Taxol®), teniposide (Vumon®), tirapazamine (Tirazone®), topotecan HCl for injection (Hycamptin®), vinblastine (Velban®), vincristine (Oncovin®), and vinorelbine (Navelbine®). In an exemplary embodiment, the chemotherapeutic agent is selected from the group consisting of anastrozole (Arimidex®), bicalutamide (Casodex®), busulfan injection (Busulfex®), cytosine arabinoside (Cytosar-U®), flutamide (Eulexin®), tezacitibine, phoenix (Yttrium90/MX-DTPA), polifeprosan 20 with carmustine implant (Gliadel®), tamoxifen citrate (Nolvadex®).

In some embodiments, a bispecific anti-CD123×anti-CD3 antibody (e.g., XmAb14045) described herein is administered to a subject in combination with one or more of the following therapeutic agents: methotrexate (e.g., Abitrexate®, Methotrexate LPF®, Mexate®, Mexate-AQ®, Folex®, Folex PFS®), nelarabine (e.g., Arranon®), doxorubicin HCl, daunorubicin in combination with cytarabine and anthracycline, or idararubicin, clofarabine (e.g., Clofarex® or Clolar®), cyclophosphamide (e.g., Cytoxan®, Neosar®, Clafen®), cytarabine (e.g., Cytosar-U®, Tarabine PFS®), dasatinib (e.g., Sprycel®), or other BCR-ABL and SRC tyrosine kinase inhibitors, Erwinaze (e.g., Asparaginase Erwinia Chrysanthemi), imatinib mesylate (e.g., Gleevec®), ponatinib HCl (e.g., Iclusig®), mercaptopurine (e.g., Purinethol®, Purixan®), pegaspargase (e.g., Oncaspar®), ponatinib HCl, prednisone, vincristine sulfate, vincristine sulfate liposome (e.g., Margibo0), vincasar PFS, and Hyper-CVAD. In an exemplary embodiment, the subject in the previous sentence has ALL.

In some embodiments, a bispecific anti-CD123×anti-CD3 antibody (e.g., XmAb14045) described herein is administered to a subject in combination with one or more of the following therapeutic agents: daunorubicin HCl (e.g., Cerubidine® or Rubidomycin®) (optionally in combination with cytarabine and an anthracycline, such as daunorubicin or idararubicin), idarubicin HCl (e.g., Idamycin®), Bcl2 inhibitor (e.g., ABT-737, venetoclax (e.g., Venclexta®)), cyclophosphamide (e.g., Cytoxan®, Clafen®, Neosar®), cytarabine (e.g., Cytosar-U®, Tarabine PFS®), doxorubicin HCl, decitabine (hypomethylating agent), fludarabine (fludara), FLT3 inhibitors (e.g., sunitinib, sorafenib, midostaurin, lestaurtinib, quizartinib, crenolanib, PLX3397), GCSF (Granulocyte-colony stimulating factor), IDH inhibitors (e.g., IDH1 inhibitors, e.g., AG120 or IDH305); IDH2 inhibitors, e.g., AG221; pan IGH1/IGH2 inhibitors, e.g., AG881), mitoxantrone HCl, thioguanine (e.g., Tabloid®), azacitidine or decitabine (e.g., hypomethylating agent), vincristine sulfate (e.g., Vincasar PFS®). In an exemplary embodiment, the subject in the previous sentence has AML.

In some embodiments, a bispecific anti-CD123×anti-CD3 antibody (e.g., XmAb14045) described herein is administered to a subject in combination with one or more of the following therapeutic agents: G100 (Immune Design), bosutinib (e.g., Bosulif®), busulfan (e.g., Busulfex®, Myleran®), cyclophosphamide (e.g., Clafen®, Cytoxan®, Neosar®), cytarabine (e.g., Cytosar-U®, Tarabine PFS®), dasatinib (e.g., Sprycel®), imatinib mesylate (e.g., Gleevec®), hydroxyurea (e.g., Hydrea®), ponatinib HCl (e.g., Iclusig®), mechlorethamine HCl (e.g., Mustargen®), nilotinib, omacetaxine mepesuccinate (e.g., Synribo®), and interferon-alpha. In an exemplary embodiment, the subject in the previous sentence has CML.

In some embodiments, a bispecific anti-CD123×anti-CD3 antibody (e.g., XmAb14045) described herein is administered to a subject in combination with CVP (a combination of cyclophosphamide, vincristine, and prednisone) and/or CHOP (a combination of cyclophosphamide, hydroxydaunorubicin, Oncovin® (vincristine), and prednisone) with or without etoposide (e.g., VP-16) and/or a combination of cyclophosphamide and pentostatin and/or a combination of chlorambucil and prednisone and/or a combination of fludarabine and cyclophosphamide and an immunomodulator such as thalidomide or a thalidomide derivative (e.g., lenalidomide).

### VIII. A3) Inhibitors, Such as Antibodies

In one embodiment, a bispecific anti-CD123×anti-CD3 antibody (e.g., XmAb14045) described herein can be used in combination with a PD1 inhibitor, a PDL1 inhibitor, a PDL2 inhibitor, a TIM3 inhibitor, a LAG3 inhibitor, a CTLA4 inhibitor, a TIGIT inhibitor, a BTLA inhibitor, a CD47 inhibitor, or a IDO inhibitor. In one embodiment, the PD1 inhibitor, PDL1 inhibitor, PDL2 inhibitor, TIM3 inhibitor, LAG3 inhibitor, CTLA4 inhibitor, TIGIT inhibitor, BTLA inhibitor, CD47 inhibitor, or IDO inhibitor is a small molecule. In one embodiment, the PD1 inhibitor, PDL1 inhibitor, PDL2 inhibitor, TIM3 inhibitor, LAG3 inhibitor, CTLA4 inhibitor, TIGIT inhibitor, BTLA inhibitor, CD47 inhibitor, or IDO inhibitor is an antibody.

In an exemplary embodiment, the anti-cancer agent is an antibody, such as an immuno-oncology agent.

### VIII. a3A) PM

In other embodiments, a bispecific anti-CD20×anti-CD3 antibody (e.g., XmAb14045) described herein can be used in combination with a PD1 inhibitor. In other embodiments, the PD1 inhibitor is a small molecule inhibitor. In other embodiments, the PD1 inhibitor is CA-170 (Curis), AUNP-12 (Aurigene), or a compound described in WO 2015/034820—in particular, BMS-1, BMS-2, BMS-79, and BMS-196.

In other embodiments, a bispecific anti-CD20×anti-CD3 antibody (e.g., XmAb14045) described herein can be used in combination with an anti-PD1 antibody. In other embodiments, the PD1 inhibitor is nivolumab (Opdivo®), pembrolizumab (Keytruda®), pidilizumab (Medivation/Pfizer), spartalizumab also known as PDR001, JNJ-63723283 (J&J), TSR-042 (Tesaro), cemiplimab also known as REGN2810 (Sanofi), AMP-224 (Amplimmune/GSK), MEDI0680 also known as AMP-514 (AstraZeneca), MGA012 (MacroGenics/Incyte), MGD013 (MacroGenics), MGD019 (MacroGenics), SHR-1210 (Shanghai Hengrui Pharma/Incyte), GLS-010 (Gloria Pharma/WuXi Biologics), JS001 (Shanghai Junshi Biosciences), tislelizumab also known as BGB-A317 (BeiGene/Celgene), sintilimab also known as IBI308 (Innovent), CX-188 (CytomX Therapeutics), or CS1003 (CStone Pharmaceuticals).

Exemplary non-limiting anti-PD1 antibody molecules are disclosed in US 2015/0210769, published on Jul. 30, 2015, entitled “Antibody Molecules to PD1 and Uses Thereof,” incorporated by reference in its entirety.

In one embodiment, the anti-PD1 antibody molecule includes at least one or two heavy chain variable domain (optionally including a constant region), at least one or two light chain variable domain (optionally including a constant region), or both, comprising the amino acid sequence of BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1; or a sequence substantially identical (e.g., at least 80%, 85%, 90%, 92%, 95%, 97%, 98%, 99% or higher identical) to any of the aforesaid sequences. The anti-PD1 antibody molecule, optionally, comprises a leader sequence from a heavy chain, a light chain, or both, as shown in Table 4 of US 2015/0210769; or a sequence substantially identical thereto.

In yet another embodiment, the anti-PD1 antibody molecule includes at least one, two, or three complementarity determining regions (CDRs) from a heavy chain variable region and/or a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the anti-PD1 antibody molecule includes at least one, two, or three CDRs (or collectively all of the CDRs) from a heavy chain variable region comprising an amino acid sequence shown in Table 1 of US 2015/0210769, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In yet another embodiment, the anti-PD1 antibody molecule includes at least one, two, or three CDRs (or collectively all of the CDRs) from a light chain variable region comprising an amino acid sequence shown in Table 1 of US 2015/0210769, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1. In certain embodiments, the anti-PD1 antibody molecule includes a substitution in a light chain CDR, e.g., one or more substitutions in a CDR1, CDR2 and/or CDR3 of the light chain. In one embodiment, the anti-PD1 antibody molecule includes a substitution in the light chain CDR3 at position 102 of the light variable region, e.g., a substitution of a cysteine to tyrosine, or a cysteine to serine residue, at position 102 of the light variable region according to Table 1 (e.g., SEQ ID NO: 16 or 24 for murine or chimeric, unmodified; or any of SEQ ID NOs: 34, 42, 46, 54, 58, 62, 66, 70, 74, or 78 for a modified sequence).

In another embodiment, the anti-PD1 antibody molecule includes at least one, two, three, four, five or six CDRs (or collectively all of the CDRs) from a heavy and light chain variable region comprising an amino acid sequence shown in Table 1 of US 2015/0210769, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In one embodiment, the anti-PD1 antibody molecule includes:

(a) a heavy chain variable region (VH) comprising a VHCDR1 amino acid sequence of SEQ ID NO: 4, a VHCDR2 amino acid sequence of SEQ ID NO: 5, and a VHCDR3 amino acid sequence of SEQ ID NO: 3; and a light chain variable region (VL) comprising a VLCDR1 amino acid sequence of SEQ ID NO: 13, a VLCDR2 amino acid sequence of SEQ ID NO: 14, and a VLCDR3 amino acid sequence of SEQ ID NO: 33, each disclosed in Table 1 of US 2015/0210769;

(b) a VH comprising a VHCDR1 amino acid sequence chosen from SEQ ID NO: 1; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 10, a VLCDR2 amino acid sequence of SEQ ID NO: 11, and a VLCDR3 amino acid sequence of SEQ ID NO: 32, each disclosed in Table 1 of US 2015/0210769;

(c) a VH comprising a VHCDR1 amino acid sequence of SEQ ID NO: 224, a VHCDR2 amino acid sequence of SEQ ID NO: 5, and a VHCDR3 amino acid sequence of SEQ ID NO: 3; and a VL comprising a VLCDR1 amino acid sequence of SEQ ID NO: 13, a VLCDR2 amino acid sequence of SEQ ID NO: 14, and a VLCDR3 amino acid sequence of SEQ ID NO: 33, each disclosed in Table 1 of US 2015/0210769; or

(d) a VH comprising a VHCDR1 amino acid sequence of SEQ ID NO: 224; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 10, a VLCDR2 amino acid sequence of SEQ ID NO: 11, and a VLCDR3 amino acid sequence of SEQ ID NO: 32, each disclosed in Table 1 of US 2015/0210769.

In another embodiment, the anti-PD1 antibody molecule comprises (i) a heavy chain variable region (VH) comprising a VHCDR1 amino acid sequence chosen from SEQ ID NO: 1, SEQ ID NO: 4, or SEQ ID NO: 224; a VHCDR2 amino acid sequence of SEQ ID NO: 2 or SEQ ID NO: 5; and a VHCDR3 amino acid sequence of SEQ ID NO: 3; and (ii) a light chain variable region (VL) comprising a VLCDR1 amino acid sequence of SEQ ID NO: 10 or SEQ ID NO: 13, a VLCDR2 amino acid sequence of SEQ ID NO: 11 or SEQ ID NO: 14, and a VLCDR3 amino acid sequence of SEQ ID NO: 32 or SEQ ID NO: 33, each disclosed in Table 1 of US 2015/0210769.

In other embodiments, the PD1 inhibitor is an anti-PD1 antibody chosen from nivolumab, pembrolizumab, or pidilizumab. In other embodiments, the PD1 inhibitor is spartalizumab (PDR001).

In some embodiments, the anti-PD1 antibody is nivolumab. Alternative names for nivolumab include MDX-1106, MDX-1106-04, ONO-4538, or BMS-936558. In some embodiments, the anti-PD1 antibody is nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168. In one embodiment, the inhibitor of PD1 is nivolumab, and having a sequence disclosed herein (or a sequence substantially identical or similar thereto, e.g., a sequence at least 85%, 90%, 95% identical or higher to the sequence specified).

The heavy and light chain amino acid sequences of nivolumab are as follows:

Heavy chain
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK
Light chain
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In some embodiments, the anti-PD1 antibody is pembrolizumab. Pembrolizumab (also referred to as lambrolizumab, MK-3475, MK03475, SCH-900475 or KEYTRUDA®; Merck) is a humanized IgG4 monoclonal antibody that binds to PD1. Pembrolizumab and other humanized anti-PD1 antibodies are disclosed in Hamid, O. et al. (2013) New England Journal of Medicine 369 (2): 134-44, U.S. Pat. No. 8,354,509 and WO2009/114335. The heavy and light chain amino acid sequences of pembrolizumab are as follows:

| Heavy chain | |
|---|---|
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTTVTVSS ASTKGPSVFP LAPCSRSTSE STAALGCLVK | 150 |
| DYFPEPVTVS WNSGALTSGV HTFPAVLQSS GLYSLSSVVT VPSSSLGTKT | 200 |
| YTCNVDHKPS NTKVDKRVES KYGPPCPPCP APEFLGGPSV FLFPPKPKDT | 250 |
| LMISRTPEVT CVVVDVSQED PEVQFNWYVD GVEVHNAKTK PREEQFNSTY | 300 |
| RVVSVLTVLH QDWLNGKEYK CKVSNKGLPS SIEKTISKAK GQPREPQVYT | 350 |
| LPPSQEEMTK NQVSLTCLVK GFYPSDIAVE WESNGQPENN YKTTPPVLDS | 400 |
| DGSFFLYSRL TVDKSRWQEG NVFSCSVMHE ALHNHYTQKS LSLSLGK | 447 |
| Light chain | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC | 218′ |

In one embodiment, the inhibitor of PD1 is pembrolizumab disclosed in, e.g., U.S. Pat. No. 8,354,509 and WO 2009/114335, and having a sequence disclosed herein (or a sequence substantially identical or similar thereto, e.g., a sequence at least 85%, 90%, 95% identical or higher to the sequence specified).

In some embodiments, the anti-PD1 antibody is pidilizumab. Pidilizumab (CT-011; Cure Tech) is a humanized IgG1k monoclonal antibody that binds to PD1. Pidilizumab and other humanized anti-PD1 monoclonal antibodies are disclosed in WO2009/101611.

Other anti-PD1 antibodies include AMP 514 (Amplimmune), among others, e.g., anti-PD1 antibodies disclosed in U.S. Pat. No. 8,609,089, US 2010028330, and/or US 20120114649.

In some embodiments, the PD1 inhibitor is an immunoadhesin (e.g., an immunoadhesin comprising an extracellular or PD1 binding portion of PDL1 or PDL2 fused to a constant region (e.g., an Fc region of an immunoglobulin sequence). In some embodiments, the PD1 inhibitor is AMP-224 (B7-DCIg; Amplimmune; e.g., disclosed in WO2010/027827 and WO2011/066342), is a PDL2 Fc fusion soluble receptor that blocks the interaction between PD1 and B7-H1.

In an exemplary embodiment, for any of the combinations of a bispecific anti-CD123×anti-CD3 antibody (e.g., XmAb14045) and PD1 inhibitor described herein, this combination further comprises another anti-cancer agent. In an exemplary embodiment, for any of the combinations of a bispecific anti-CD123×anti-CD3 antibody (e.g., XmAb14045) and PD1 inhibitor described herein, this combination further comprises a chemotherapeutic. In an exemplary embodiment, for any of the combinations of a bispecific anti-CD123×anti-CD3 antibody (e.g., XmAb14045) and PD1 inhibitor described herein, this combination further comprises a pyrimidine analog. In an exemplary embodiment, for any of the combinations of a bispecific anti-CD123×anti-CD3 antibody (e.g., XmAb14045) and PD1 inhibitor described herein, this combination further comprises cytarabine. In an exemplary embodiment, for any of the combinations of a bispecific anti-CD123×anti-CD3 antibody (e.g., XmAb14045) and PD1 inhibitor described herein, this combination further comprises anthracycline. In an exemplary embodiment, for any of the combinations of a bispecific anti-CD123×anti-CD3 antibody (e.g., XmAb14045) and PD1 inhibitor described herein, this combination further comprises idarubicin. In an exemplary embodiment, for any of the combinations of a bispecific anti-CD123×anti-CD3 antibody (e.g., XmAb14045) and PD1 inhibitor described herein, this combination further comprises daunorubicin. In an exemplary embodiment, for any of the combinations of a bispecific anti-CD123×anti-CD3 antibody (e.g., XmAb14045) and PD1 inhibitor described herein, this combination further comprises anthracenedione. In an exemplary embodiment, for any of the combinations of a bispecific anti-CD123×anti-CD3 antibody (e.g., XmAb14045) and PD1 inhibitor described herein, this combination further comprises gemtuzumab. In an exemplary embodiment, for any of the combinations of a bispecific anti-CD123×anti-CD3 antibody (e.g., XmAb14045) and PD1 inhibitor described herein, this combination further comprises a FLT3 inhibitor. In an exemplary embodiment, for any of the combinations of a bispecific anti-CD123×anti-CD3 antibody (e.g., XmAb14045) and PD1 inhibitor described herein, this combination further comprises a topoisomerase inhibitor. In an exemplary embodiment, for any of the combinations of a bispecific anti-CD123×anti-CD3 antibody (e.g., XmAb14045) and PD1 inhibitor described herein, this combination further comprises a topoisomerase II inhibitor. In an exemplary embodiment, for any of the combinations of a bispecific anti-CD123×anti-CD3 antibody (e.g., XmAb14045) and PD1 inhibitor described herein, this combination further comprises etoposide. In an exemplary embodiment, for any of the combinations of a bispecific anti-CD123×anti-CD3 antibody (e.g., XmAb14045) and PD1 inhibitor described herein, this combination further comprises mitoxantrone. In an exemplary embodiment, for any of the combinations of a bispecific anti-CD123×anti-CD3 antibody (e.g., XmAb14045) and PD1 inhibitor described herein, this combination further comprises an adenosine analog. In an exemplary embodiment, for any of the combinations of a bispecific anti-CD123×anti-CD3 antibody (e.g., XmAb14045) and PD1 inhibitor described herein, this combination further comprises fludarabine. In an exemplary embodiment, for any of the combinations of a bispecific anti-CD123×anti-CD3 antibody (e.g., XmAb14045) and PD1 inhibitor described herein, this combination further comprises cladribine. In an exemplary embodiment, for any of the combinations of a bispecific anti-CD123×anti-CD3 antibody (e.g., XmAb14045) and PD1 inhibitor described herein, this combination further comprises a kinase inhibitor. In an exemplary embodiment, for any of the combinations of a bispecific anti-CD123×anti-CD3 antibody (e.g., XmAb14045) and PD1 inhibitor described herein, this combination further comprises a Bcr-Abl inhibitor. In an exemplary embodiment, for any of the combinations of a bispecific anti-CD123×anti-CD3 antibody (e.g., XmAb14045) and PD1 inhibitor described herein, this combination further comprises imatinib or nilotinib or dasatinib or bosutinib or ponatinib or a combination thereof. In an exemplary embodiment, for any of the combinations of a bispecific anti-CD123×anti-CD3 antibody (e.g., XmAb14045) and PD1 inhibitor described herein, this combination further comprises omacetaxine. In an exemplary embodiment, for any of the combinations described in this paragraph, the PD1 inhibitor is spartalizumab.

### VIII. a3B) PDL1 or PDL2

In one embodiment, a bispecific anti-CD123×anti-CD3 antibody (e.g., XmAb14045) described herein can be used in combination with a PDL1 inhibitor. In one embodiment, a bispecific anti-CD123×anti-CD3 antibody (e.g., XmAb14045) described herein can be used in combination with a PDL2 inhibitor.

In some embodiments, the PDL1 inhibitor is an antibody molecule. In some embodiments, the anti-PDL1 inhibitor is atezolizumab (Tecentriq®) formerly known as YW243.55.S70 or MPDL3280A, avelumab (Bavencio® (EMD Serono) formerly known as MSB-0010718C, durvalumab (Imfinzi®; MedImmune/AstraZeneca) formerly known as MEDI-4736, FAZ053, LY3300054 (Lilly), ABBV-181 (AbbVie), MSB2311 (MabSpace Biosciences), MDX-1105 also known as BMS-936559, CS1001 formerly known as WBP3155 (CStone Pharmaceuticals), KNO35 (Alphamab), CA-327 (Curis), CX-072 (CytomX Therapeutics), M7824 (EMD Serono), HTI-1316 (Hengrui Therapeutics), or JS003 (Shanghai Junshi Biosciences).

Exemplary non-limiting PDL1 inhibitors are disclosed in US 2016/0108123, published on Apr. 21, 2016, entitled “Antibody Molecules to PDL1 and Uses Thereof,” incorporated by reference in its entirety.

In one embodiment, the PDL1 inhibitor includes at least one or two heavy chain variable domain (optionally including a constant region), at least one or two light chain variable domain (optionally including a constant region), or both, comprising the amino acid sequence of any of BAP058-hum01, BAP058-hum02, BAP058-hum03, BAP058-hum04, BAP058-hum05, BAP058-hum06, BAP058-hum07, BAP058-hum08, BAP058-hum09, BAP058-hum10, BAP058-hum11, BAP058-hum12, BAP058-hum13, BAP058-hum14, BAP058-hum15, BAP058-hum16, BAP058-hum17, BAP058-Clone-K, BAP058-Clone-L, BAP058-Clone-M, BAP058-Clone-N, or BAP058-Clone-O; or as described in Table 1 of US 2016/0108123, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the PDL1 inhibitor includes at least one, two, or three complementarity determining regions (CDRs) from a heavy chain variable region and/or a light chain variable region of an antibody described herein, e.g., an antibody chosen from any of BAP058-hum01, BAP058-hum02, BAP058-hum03, BAP058-hum04, BAP058-hum05, BAP058-hum06, BAP058-hum07, BAP058-hum08, BAP058-hum09, BAP058-hum10, BAP058-hum11, BAP058-hum12, BAP058-hum13, BAP058-hum14, BAP058-hum15, BAP058-hum16, BAP058-hum17, BAP058-Clone-K, BAP058-Clone-L, BAP058-Clone-M, BAP058-Clone-N, or BAP058-Clone-O; or as described in Table 1 of US 2016/0108123, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the PDL1 inhibitor includes at least one, two, or three CDRs (or collectively all of the CDRs) from a heavy chain variable region comprising an amino acid sequence shown in Table 1 of US 2016/0108123, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In yet another embodiment, the PDL1 inhibitor includes at least one, two, or three CDRs (or collectively all of the CDRs) from a light chain variable region comprising an amino acid sequence shown in Table 1 of US 2016/0108123, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1. In certain embodiments, the PDL1 inhibitor includes a substitution in a light chain CDR, e.g., one or more substitutions in a CDR1, CDR2 and/or CDR3 of the light chain.

In another embodiment, the PDL1 inhibitor includes at least one, two, three, four, five or six CDRs (or collectively all of the CDRs) from a heavy and light chain variable region comprising an amino acid sequence shown in Table 1, or encoded by a nucleotide sequence shown in Table 1 of US 2016/0108123. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In one embodiment, the PDL1 inhibitor includes:

(i) a heavy chain variable region (VH) including a VHCDR1 amino acid sequence chosen from SEQ ID NO: 1, SEQ ID NO: 4 or SEQ ID NO: 195; a VHCDR2 amino acid sequence of SEQ ID NO: 2; and a VHCDR3 amino acid sequence of SEQ ID NO: 3, each disclosed in Table 1 of US 2016/0108123; and

(ii) a light chain variable region (VL) including a VLCDR1 amino acid sequence of SEQ ID NO: 9, a VLCDR2 amino acid sequence of SEQ ID NO: 10, and a VLCDR3 amino acid sequence of SEQ ID NO: 11, each disclosed in Table 1 of US 2016/0108123.

In another embodiment, the PDL1 inhibitor includes:

(i) a heavy chain variable region (VH) including a VHCDR1 amino acid sequence chosen from SEQ ID NO: 1, SEQ ID NO: 4 or SEQ ID NO: 195; a VHCDR2 amino acid sequence of SEQ ID NO: 5, and a VHCDR3 amino acid sequence of SEQ ID NO: 3, each disclosed in Table 1 of US 2016/0108123; and

(ii) a light chain variable region (VL) including a VLCDR1 amino acid sequence of SEQ ID NO: 12, a VLCDR2 amino acid sequence of SEQ ID NO: 13, and a VLCDR3 amino acid sequence of SEQ ID NO: 14, each disclosed in Table 1 of US 2016/0108123.

In one embodiment, the PDL1 inhibitor comprises the VHCDR1 amino acid sequence of SEQ ID NO: 1. In another embodiment, the anti-PDL1 antibody molecule comprises the VHCDR1 amino acid sequence of SEQ ID NO: 4. In yet another embodiment, the PDL1 inhibitor comprises the VHCDR1 amino acid sequence of SEQ ID NO: 195, each disclosed in Table 1 of US 2016/0108123.

In some embodiments, the PDL1 inhibitor is MSB0010718C. MSB0010718C (also referred to as A09-246-2; Merck Serono) is a monoclonal antibody that binds to PDL1. Pembrolizumab and other humanized anti-PDL1 antibodies are disclosed in WO2013/079174, and having a sequence disclosed herein (or a sequence substantially identical or similar thereto, e.g., a sequence at least 85%, 90%, 95% identical or higher to the sequence specified). The heavy and light chain amino acid sequences of MSB0010718C include at least the following:

Heavy chain (SEQ ID NO: 24 as disclosed in
WO2013/079174)
EVQLLESGGGLVQPGGSLRLSCAASGFTFSSYIMMWVRQAPGKGLEWVSS
IYPSGGITFYADKGRFTISRDNSKNTLYLQMNSLRAEDTAVYYCARIKLG
TVTTVDYWGQGTLVTVSS
Light chain (SEQ ID NO: 25 as disclosed in
WO2013/079174)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVL.

In one embodiment, the PDL1 inhibitor is YW243.55.S70. The YW243.55.S70 antibody is an anti-PDL1 described in WO 2010/077634 (heavy and light chain variable region sequences shown in SEQ ID Nos. 20 and 21, respectively), and having a sequence disclosed therein (or a sequence substantially identical or similar thereto, e.g., a sequence at least 85%, 90%, 95% identical or higher to the sequence specified).

In one embodiment, the PDL1 inhibitor is MDX-1105. MDX-1105, also known as BMS-936559, is an anti-PDL1 antibody described in WO2007/005874, and having a sequence disclosed therein (or a sequence substantially identical or similar thereto, e.g., a sequence at least 85%, 90%, 95% identical or higher to the sequence specified).

In one embodiment, the PDL1 inhibitor is MDPL3280A (Genentech/Roche). MDPL3280A is a human Fc optimized IgG1 monoclonal antibody that binds to PDL1. MDPL3280A and other human monoclonal antibodies to PDL1 are disclosed in U.S. Pat. No. 7,943,743 and U.S. Publication No.: 20120039906.

In other embodiments, the PDL2 inhibitor is AMP-224. AMP-224 is a PDL2 Fc fusion soluble receptor that blocks the interaction between PD1 and B7-H1 (B7-DCIg; Amplimmune; e.g., disclosed in WO2010/027827 and WO2011/066342).

In an exemplary embodiment, for any of the combinations of a bispecific anti-CD123×anti-CD3 antibody (e.g., XmAb14045) and PDL1 inhibitor described herein, this combination further comprises another anti-cancer agent. In an exemplary embodiment, for any of the combinations of a bispecific anti-CD123×anti-CD3 antibody (e.g., XmAb14045) and PDL1 inhibitor described herein, this combination further comprises a chemotherapeutic. In an exemplary embodiment, for any of the combinations of a bispecific anti-CD123×anti-CD3 antibody (e.g., XmAb14045) and PDL1 inhibitor described herein, this combination further comprises a pyrimidine analog. In an exemplary embodiment, for any of the combinations of a bispecific anti-CD123×anti-CD3 antibody (e.g., XmAb14045) and PDL1 inhibitor described herein, this combination further comprises cytarabine. In an exemplary embodiment, for any of the combinations of a bispecific anti-CD123×anti-CD3 antibody (e.g., XmAb14045) and PDL1 inhibitor described herein, this combination further comprises anthracycline. In an exemplary embodiment, for any of the combinations of a bispecific anti-CD123×anti-CD3 antibody (e.g., XmAb14045) and PDL1 inhibitor described herein, this combination further comprises idarubicin. In an exemplary embodiment, for any of the combinations of a bispecific anti-CD123×anti-CD3 antibody (e.g., XmAb14045) and PDL1 inhibitor described herein, this combination further comprises daunorubicin. In an exemplary embodiment, for any of the combinations of a bispecific anti-CD123×anti-CD3 antibody (e.g., XmAb14045) and PDL1 inhibitor described herein, this combination further comprises anthracenedione. In an exemplary embodiment, for any of the combinations of a bispecific anti-CD123×anti-CD3 antibody (e.g., XmAb14045) and PDL1 inhibitor described herein, this combination further comprises gemtuzumab. In an exemplary embodiment, for any of the combinations of a bispecific anti-CD123×anti-CD3 antibody (e.g., XmAb14045) and PDL1 inhibitor described herein, this combination further comprises a FLT3 inhibitor. In an exemplary embodiment, for any of the combinations of a bispecific anti-CD123×anti-CD3 antibody (e.g., XmAb14045) and PDL1 inhibitor described herein, this combination further comprises a topoisomerase inhibitor. In an exemplary embodiment, for any of the combinations of a bispecific anti-CD123×anti-CD3 antibody (e.g., XmAb14045) and PDL1 inhibitor described herein, this combination further comprises a topoisomerase II inhibitor. In an exemplary embodiment, for any of the combinations of a bispecific anti-CD123×anti-CD3 antibody (e.g., XmAb14045) and PDL1 inhibitor described herein, this combination further comprises etoposide. In an exemplary embodiment, for any of the combinations of a bispecific anti-CD123×anti-CD3 antibody (e.g., XmAb14045) and PDL1 inhibitor described herein, this combination further comprises mitoxantrone. In an exemplary embodiment, for any of the combinations of a bispecific anti-CD123×anti-CD3 antibody (e.g., XmAb14045) and PDL1 inhibitor described herein, this combination further comprises an adenosine analog. In an exemplary embodiment, for any of the combinations of a bispecific anti-CD123×anti-CD3 antibody (e.g., XmAb14045) and PDL1 inhibitor described herein, this combination further comprises fludarabine. In an exemplary embodiment, for any of the combinations of a bispecific anti-CD123×anti-CD3 antibody (e.g., XmAb14045) and PDL1 inhibitor described herein, this combination further comprises cladribine. In an exemplary embodiment, for any of the combinations of a bispecific anti-CD123×anti-CD3 antibody (e.g., XmAb14045) and PDL1 inhibitor described herein, this combination further comprises a kinase inhibitor. In an exemplary embodiment, for any of the combinations of a bispecific anti-CD123×anti-CD3 antibody (e.g., XmAb14045) and PDL1 inhibitor described herein, this combination further comprises a Bcr-Abl inhibitor. In an exemplary embodiment, for any of the combinations of a bispecific anti-CD123×anti-CD3 antibody (e.g., XmAb14045) and PDL1 inhibitor described herein, this combination further comprises imatinib or nilotinib or dasatinib or bosutinib or ponatinib or a combination thereof. In an exemplary embodiment, for any of the combinations of a bispecific anti-CD123×anti-CD3 antibody (e.g., XmAb14045) and PDL1 inhibitor described herein, this combination further comprises omacetaxine. In an exemplary embodiment, for any of the combinations described in this paragraph, this combination further comprises a PD1 inhibitor. In an exemplary embodiment, for any of the combinations described in this paragraph, the PD1 inhibitor is spartalizumab.

### VIII. a3C) TIM3

In one embodiment, a bispecific anti-CD123×anti-CD3 antibody (e.g., XmAb14045) described herein can be used in combination with a TIM3 inhibitor. In an exemplary embodiment, the TIM3 inhibitor is MGB453, INCAGN2390 (Incyte), Sym023, TSR-022 (Tesaro), and LY3321367 (Lilly).

Exemplary non-limiting TIM3 inhibitors are disclosed in US 2015/0218274, published on Aug. 6, 2015, entitled “Antibody Molecules to TIM3 and Uses Thereof,” incorporated by reference in its entirety.

In one embodiment, the TIM3 inhibitor includes at least one or two heavy chain variable domain (optionally including a constant region), at least one or two light chain variable domain (optionally including a constant region), or both, comprising the amino acid sequence of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences. The TIM3 inhibitor, optionally, comprises a leader sequence from a heavy chain, a light chain, or both, as shown in US 2015/0218274; or a sequence substantially identical thereto.

In yet another embodiment, the TIM3 inhibitor includes at least one, two, or three complementarity determining regions (CDRs) from a heavy chain variable region and/or a light chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In yet another embodiment, the TIM3 inhibitor includes at least one, two, or three CDRs (or collectively all of the CDRs) from a heavy chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Table 1-4.

In yet another embodiment, the TIM3 inhibitor includes at least one, two, or three CDRs (or collectively all of the CDRs) from a light chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Tables 1-4. In certain embodiments, the TIM3 inhibitor includes a substitution in a light chain CDR, e.g., one or more substitutions in a CDR1, CDR2 and/or CDR3 of the light chain.

In another embodiment, the TIM3 inhibitor includes at least one, two, three, four, five or six CDRs (or collectively all of the CDRs) from a heavy and light chain variable region comprising an amino acid sequence shown in Tables 1-4 of US 2015/0218274, or encoded by a nucleotide sequence shown in Tables 1-4. In one embodiment, one or more of the CDRs (or collectively all of the CDRs) have one, two, three, four, five, six or more changes, e.g., amino acid substitutions or deletions, relative to the amino acid sequence shown in Tables 1-4, or encoded by a nucleotide sequence shown in Tables 1-4.

In one embodiment, the TIM3 inhibitor includes:

(a) a heavy chain variable region (VH) comprising a VHCDR1 amino acid sequence chosen from SEQ ID NO: 9; a VHCDR2 amino acid sequence of SEQ ID NO: 10; and a VHCDR3 amino acid sequence of SEQ ID NO: 5; and a light chain variable region (VL) comprising a VLCDR1 amino acid sequence of SEQ ID NO: 12, a VLCDR2 amino acid sequence of SEQ ID NO: 13, and a VLCDR3 amino acid sequence of SEQ ID NO: 14, each disclosed in Tables 1-4 of US 2015/0218274;

(b) a VH comprising a VHCDR1 amino acid sequence chosen from SEQ ID NO: 3; a VHCDR2 amino acid sequence of SEQ ID NO: 4; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8, each disclosed in Tables 1-4 of US 2015/0218274;

(c) a VH comprising a VHCDR1 amino acid sequence chosen from SEQ ID NO: 9; a VHCDR2 amino acid sequence of SEQ ID NO: 25; and a VHCDR3 amino acid sequence of SEQ ID NO: 5; and a VL comprising a VLCDR1 amino acid sequence of SEQ ID NO: 12, a VLCDR2 amino acid sequence of SEQ ID NO: 13, and a VLCDR3 amino acid sequence of SEQ ID NO: 14, each disclosed in Tables 1-4 of US 2015/0218274;

(d) a VH comprising a VHCDR1 amino acid sequence chosen from SEQ ID NO: 3; a VHCDR2 amino acid sequence of SEQ ID NO: 24; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8, each disclosed in Tables 1-4 of US 2015/0218274;

(e) a VH comprising a VHCDR1 amino acid sequence chosen from SEQ ID NO: 9; a VHCDR2 amino acid sequence of SEQ ID NO: 31; and a VHCDR3 amino acid sequence of SEQ ID NO: 5; and a VL comprising a VLCDR1 amino acid sequence of SEQ ID NO: 12, a VLCDR2 amino acid sequence of SEQ ID NO: 13, and a VLCDR3 amino acid sequence of SEQ ID NO: 14, each disclosed in Tables 1-4 of US 2015/0218274; or

(f) a VH comprising a VHCDR1 amino acid sequence chosen from SEQ ID NO: 3; a VHCDR2 amino acid sequence of SEQ ID NO: 30; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8, each disclosed in Tables 1-4 of US 2015/0218274.

Exemplary TIM3 inhibitor are disclosed in U.S. Pat. No. 8,552,156, WO 2011/155607, EP 2581113 and U.S. Publication No.: 2014/044728.

### VIII. a3D) LAG3

In one embodiment, a bispecific anti-CD123×anti-CD3 antibody (e.g., XmAb14045) described herein can be used in combination with a LAG3 inhibitor. In one embodiment, the LAG3 Inhibitor is LAG525, TSR-033 (Tesaro), REGN3767 (Sanofi), eftilagimod alpha also known as IMP321 (Prima BioMed), MGD013 (MacroGenics), FS118 (F-star/Merck), INCAGN2385 (Incyte), or GSK2831781 (GSK).

Exemplary non-limiting LAG3 inhibitors are disclosed in US 2015/0259420 published on Sep. 17, 2015, entitled “Antibody Molecules to LAG3 and Uses Thereof,” incorporated by reference in its entirety.

In one embodiment, the LAG3 inhibitor includes at least one or two heavy chain variable domain (optionally including a constant region), at least one or two light chain variable domain (optionally including a constant region), or both, comprising the amino acid sequence of any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050(Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the LAG3 inhibitor includes at least one, two, or three complementarity determining regions (CDRs) from a heavy chain variable region and/or a light chain variable region of an antibody described herein, e.g., an antibody chosen from any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050(Ser) (e.g., BAP050-hum0l-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the LAG3 inhibitor includes at least one, two, or three CDRs (or collectively all of the CDRs) from a heavy chain variable region comprising an amino acid sequence shown in Table 1 of US 2015/0259420, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In yet another embodiment, the LAG3 inhibitor includes at least one, two, or three CDRs (or collectively all of the CDRs) from a light chain variable region comprising an amino acid sequence shown in Table 1 of US 2015/0259420, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1. In certain embodiments, the anti-PDL1 antibody molecule includes a substitution in a light chain CDR, e.g., one or more substitutions in a CDR1, CDR2 and/or CDR3 of the light chain.

In another embodiment, the LAG3 inhibitor includes at least one, two, three, four, five or six CDRs (or collectively all of the CDRs) from a heavy and light chain variable region comprising an amino acid sequence shown in Table 1, or encoded by a nucleotide sequence shown in Table 1 of US 2015/0259420. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

In one embodiment, the LAG3 inhibitor includes:

(i) a heavy chain variable region (VH) including a VHCDR1 amino acid sequence chosen from SEQ ID NO: 1, SEQ ID NO: 4 or SEQ ID NO: 286; a VHCDR2 amino acid sequence of SEQ ID NO: 2; and a VHCDR3 amino acid sequence of SEQ ID NO: 3, each disclosed in Table 1 of US 2015/0259420; and

(ii) a light chain variable region (VL) including a VLCDR1 amino acid sequence of SEQ ID NO: 10, a VLCDR2 amino acid sequence of SEQ ID NO: 11, and a VLCDR3 amino acid sequence of SEQ ID NO: 12, each disclosed in Table 1 of US 2015/0259420.

In another embodiment, the anti-LAG3 antibody molecule includes:

(i) a heavy chain variable region (VH) including a VHCDR1 amino acid sequence chosen from SEQ ID NO: 1, SEQ ID NO: 4 or SEQ ID NO: 286; a VHCDR2 amino acid sequence of SEQ ID NO: 5, and a VHCDR3 amino acid sequence of SEQ ID NO: 3, each disclosed in Table 1 of US 2015/0259420; and

(ii) a light chain variable region (VL) including a VLCDR1 amino acid sequence of SEQ ID NO: 13, a VLCDR2 amino acid sequence of SEQ ID NO: 14, and a VLCDR3 amino acid sequence of SEQ ID NO: 15, each disclosed in Table 1 of US 2015/0259420.

In one embodiment, the anti-LAG3 antibody molecule comprises the VHCDR1 amino acid sequence of SEQ ID NO: 1. In another embodiment, the anti-LAG3 antibody molecule comprises the VHCDR1 amino acid sequence of SEQ ID NO: 4. In yet another embodiment, the anti-LAG3 antibody molecule comprises the VHCDR1 amino acid sequence of SEQ ID NO: 286, each disclosed in Table 1 of US 2015/0259420.

In some embodiments, the anti-LAG3 antibody is relatlimab. Relatlimab (also referred to as BMS-986016 or BMS986016; Bristol-Myers Squibb) is a monoclonal antibody that binds to LAG3. Relatlimab and other humanized anti-LAG3 antibodies are disclosed in US 2011/0150892, WO2010/019570, and WO2014/008218.

### VIII. a3E) CTLA4

In one embodiment, a bispecific anti-CD123×anti-CD3 antibody (e.g., XmAb14045) described herein can be used in combination with a CTLA4 inhibitor.

Exemplary anti-CTLA4 antibodies include tremelimumab (IgG2 monoclonal antibody available from Medlmmune, a subsidiary of AstraZeneca, formerly known as ticilimumab, CP-675,206); and ipilimumab (Yervoy®) (CTLA4 antibody, also known as MDX-010, CAS No. 477202-00-9). Other exemplary anti-CTLA4 antibodies are disclosed, e.g., in U.S. Pat. No. 5,811,097. Other exemplary anti-CTLA4 antibodies include abatacept (Orencia®), IBI310 (Innovent), BMS-986249 (BMS/CytomX Therapeutics), or CS1002 (CStone Pharmaceuticals).

In one embodiment, a bispecific anti-CD123×anti-CD3 antibody (e.g., XmAb14045) described herein can be used in combination with an anti-PD1 antibody molecule, e.g., as described herein, and an anti-CTLA4 antibody, e.g., ipilimumab.

### VIII. a3F) TIGIT

In one embodiment, a bispecific anti-CD20×anti-CD3 antibody (e.g., XmAb14045) described herein can be used in combination with a TIGIT inhibitor. In an exemplary embodiment, the TIGIT inhibitor is OMP-313M32 (OncoMed).

### VIII. a3G) BTLA

In one embodiment, a bispecific anti-CD20×anti-CD3 antibody (e.g., XmAb14045) described herein can be used in combination with a BTLA inhibitor.

### VIII. a3H) CD47

In one embodiment, a bispecific anti-CD20×anti-CD3 antibody (e.g., XmAb14045) described herein can be used in combination with a CD47 inhibitor. In an exemplary embodiment, the CD47 inhibitor is TTI-621 (Trillium Therapeutics), TTI-622 (Trillium Therapeutics), Hu5F9-G4 (Forty-Seven), or CC-90002 (InhibRx/Celgene).

### VIII. a31) IDO

In one embodiment, a bispecific anti-CD20×anti-CD3 antibody (e.g., XmAb14045) described herein can be used in combination with an IDO inhibitor. In an exemplary embodiment, the IDO inhibitor is navoximod also known as GDC-0919 (Genetech/NewLink Genetics), indoximod or prodrugs of indoximod such as NLG802 (NewLink Genetics), epacadostat also known as INCB024360 (Incyte), HTI-1090 also known as SHR9146 (Hengrui Therapeutics), BMS-986205 (BMS), or LY3381916 (Lilly).

### VIII. a3J) GITR Agonist

In one embodiment, a bispecific anti-CD123×anti-CD3 antibody (e.g., XmAb14045) described herein can be used in combination with a GITR agonist.

In one embodiment, a bispecific anti-CD20×anti-CD3 antibody (e.g., XmAb13676) described herein can be used in combination with a GITR agonist. In an exemplary embodiment, the GITR inhibitor is TRX518-001, GWN323, MEDI1873 (MedImmune), OMP-336B11 (OncoMed), or ICAGN01876 (Incyte).

Exemplary GITR agonists include, e.g., GITR fusion proteins and anti-GITR antibodies (e.g., bivalent anti-GITR antibodies), such as, a GITR fusion protein described in U.S. Pat. No. 6,111,090, European Patent No.: 0920505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, U.S. Pat. No. 8,709,424, PCT Publication No.: WO 2013/039954, International Publication No.: WO2013/039954, U.S. Publication No.: US2014/0072566, International Publication NO.: WO2015/026684, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, U.S. Pat. No. 6,689,607, PCT Publication No.: WO2006/083289, PCT Publication No.: WO 2005/115451, U.S. Pat. No. 7,618,632, PCT Publication No.: WO 2011/051726, International Publication No.: WO2004060319, and International Publication No.: WO2014012479.

In one embodiment, a bispecific anti-CD123×anti-CD3 antibody (e.g., XmAb14045) described herein can be used in combination with a GITR agonist and a PD1 inhibitor, e.g., as described in WO2015/026684.

In another embodiment, a bispecific anti-CD123×anti-CD3 antibody (e.g., XmAb14045) described herein can be used in combination with a GITR agonist and a TLR agonist, e.g., as described in WO2004060319, and International Publication No.: WO2014012479.

In one embodiment, a bispecific anti-CD20×anti-CD3 antibody (e.g., XmAb14045) described herein can be used in combination with a GITR agonist and a PD1 inhibitor, e.g., as described in WO2015/026684.

In another embodiment, a bispecific anti-CD20×anti-CD3 antibody (e.g., XmAb14045) described herein can be used in combination with a GITR agonist and a TLR agonist, e.g., as described in WO2004060319, and International Publication No.: WO2014012479.

### VIII. a3K) ICOS Agonist

In one embodiment, a bispecific anti-CD20×anti-CD3 antibody (e.g., XmAb14045) described herein can be used in combination with an ICOS agonist.

### VIII. b) Side-Effect Ameliorating Agent

In some embodiments, a bispecific anti-CD123×anti-CD3 antibody (e.g., XmAb14045) described herein is administered to a subject with a side-effect ameliorating agent. Side effects associated with the administration of a bispecific anti-CD123×anti-CD3 antibody (e.g., XmAb14045) include, but are not limited to cytokine release syndrome (“CRS”). Other possible side effects include hemophagocytic lymphohistiocytosis (HLH), also termed Macrophage Activation Syndrome (MAS). Symptoms of CRS may include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardiac output (early) and potentially diminished cardiac output. CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures.

In an exemplary embodiment, the side-effect ameliorating agent is selected from the group consisting of: steroids, antihistamines, anti-allergic agents, antinausea agents (or anti-emetics), analgesic agents, antipyretic agents, cytoprotective agents, vasopressor agents, anticonvulsant agents, antiinflammatories, and combinations thereof

### VIII. b1) Steroid

In an exemplary embodiment, the side-effect ameliorating agent is a steroid. In an exemplary embodiment, the steroid is a corticosteroid. In an exemplary embodiment, the corticosteroid is a glucorticoid. In an exemplary embodiment, the corticosteroid is selected from the group consisting of betamethasone, dexamethasone, prednisone, prednisolone, methylprednisolone, and triamcinolone. In an exemplary embodiment, the corticosteroid is selected from the group consisting of hydrocortisone, cortisone, and ethamethasoneb. In an exemplary embodiment, the steroid is fludrocortisone.

### VIII. b2) Antihistamine

In an exemplary embodiment, the side-effect ameliorating agent is an antihistamine. In an exemplary embodiment, the antihistamine is an H₁ antagonist. In an exemplary embodiment, the H₁ antagonist is selected from the group consisting of acrivastine, azelastine, bilastine, bromodiphenhydramine, brompheniramine, buclizine, carbinoxamine, cetirizine (Zyrtec®), chlorodiphenhydramine, chlorphenamine, clemastine, cyclizine, cyproheptadine, dexbrompheniramine, dexchlorpheniramine, dimenhydrinate, dimetindene, diphenhydramine, doxylamine, ebastine, embramine, fexofenadine (Allegra®), hydroxyzine (Vistaril®), loratadine (Claritin®), meclizine, mirtazapine, olopatadine, orphenadrine, phenindamine, pheniramine, phenyltoloxamine, promethazine, quetiapine (Seroquel®), rupatadine (Alergoliber®), tripelennamine, and triprolidine.

In an exemplary embodiment, the antihistamine is acrivastine. In an exemplary embodiment, the antihistamine is cetirizine. In an exemplary embodiment, the antihistamine is diphenhydramine. In an exemplary embodiment, the antihistamine is Benadryl®.

In an exemplary embodiment, the antihistamine is an H₁ inverse agonist. In an exemplary embodiment, the H₁ inverse agonist is selected from the group consisting of acrivastine, cetirizine, levocetirizine, desloratadine, and pyrilamine.

In an exemplary embodiment, the antihistamine is an H2 antihistamine. In an exemplary embodiment, the H2 antihistamine is an H2 antagonist. In an exemplary embodiment, the H2 antihistamine is an H2 inverse agonist. In an exemplary embodiment, the H2 antihistamine is selected from the group consisting of cimetidine, famotidine, lafutidine, nizatidine, ranitidine, roxatidine, and tiotidine.

### VIII. b3) Anti-Allergy Agent

In an exemplary embodiment, the side-effect ameliorating agent is an antiallergy agent. In an exemplary embodiment, the side-effect ameliorating agent is selected from the group consisting of antihistamines, glucocorticoids, epinephrine (adrenaline), mast cell stabilizers, antileukotriene agents, anti-cholinergics, and decongestants. In an exemplary embodiment, the side-effect ameliorating agent is a decongestant. In an exemplary embodiment, the side-effect ameliorating agent is an adrenaline releasing agent. In an exemplary embodiment, the side-effect ameliorating agent is levomethamphetamine, phenylpropanolamine, propylhexedrine (Benzedrex®), or loratadine. In an exemplary embodiment, the side-effect ameliorating agent is an α-adrenergic receptor agonist. In an exemplary embodiment, the side-effect ameliorating agent is naphazoline, oxymetazoline, phenylephrine, synephrine, tetryzoline, tramazoline, or xylometazoline.

### VIII. b4) Antinausea Agents (or Anti-Emetic)

In an exemplary embodiment, the side-effect ameliorating agent is an antinausea agent. In an exemplary embodiment, the side-effect ameliorating agent is an antiemetic agent. In an exemplary embodiment, the side-effect ameliorating agent is a 5-HT3 receptor antagonist. In an exemplary embodiment, the side-effect ameliorating agent is a dolasetron (Anzemet®), granisetron (Kytril®, Sancuso®), ondansetron (Zofran®), tropisetron (Setrovel®, Navoban®), palonosetron (Aloxi®), mirtazapine (Remeron®). In an exemplary embodiment, the side-effect ameliorating agent is a dopamine antagonist. In an exemplary embodiment, the side-effect ameliorating agent is a 5-HT3 receptor antagonist. In an exemplary embodiment, the side-effect ameliorating agent is domperidone (Motilium®), olanzapine (Zyprexa®), droperidol, haloperidol, chlorpromazine, prochlorperazine, alizapride, prochlorperazine (Compazine®, Stemzine®, Buccastem®, Stemetil®, Phenotil®), metoclopramide (Reglan®). In an exemplary embodiment, the side-effect ameliorating agent is a NK1 receptor antagonist. In an exemplary embodiment, the side-effect ameliorating agent is aprepitant (Emend®), casopitant, rolapitant (Varubi®). In an exemplary embodiment, the side-effect ameliorating agent is an anticholinergic. In an exemplary embodiment, the side-effect ameliorating agent is scopolamine.

### VIII. b5) Analgesic and/or Antipyretic Agent

In an exemplary embodiment, the side-effect ameliorating agent is an analgesic agent. In an exemplary embodiment, the side-effect ameliorating agent is an antipyretic agent. In an exemplary embodiment, the side-effect ameliorating agent is a salicylate, or a derivative thereof. In an exemplary embodiment, the salicylate is selected from the group consisting of aspirin, diflunisal, salsalate, and salicylic acid, or a derivative thereof. In an exemplary embodiment, the salicylate is selected from the group consisting of choline salicylate, magnesium salicylate, and sodium salicylate. In an exemplary embodiment, the side-effect ameliorating agent agent is aspirin. In an exemplary embodiment, the side-effect ameliorating agent is acetaminophen, or a derivative thereof. In an exemplary embodiment, the side-effect ameliorating agent is an NSAID, or a derivative thereof. In an exemplary embodiment, the NSAID is a propionic acid derivative. In an exemplary embodiment, the NSAID is selected from the group consisting of ibuprofen, dexibuprofen, naproxen, fenoprofen, ketoprofen, dexketoprofen, flurbiprofen, oxaprozin, loxoprofen, or a derivative thereof. In an exemplary embodiment, the NSAID is ibuprofen. In an exemplary embodiment, the NSAID is naproxen. In an exemplary embodiment, the NSAID is an acetic acid derivative. In an exemplary embodiment, the NSAID is selected from the group consisting of indomethacin, tolmetin, sulindac, etodolac, ketorolac, diclofenac, aceclofenac, nabumetone, or a derivative thereof. In an exemplary embodiment, the NSAID is an enolic acid derivative. In an exemplary embodiment, the NSAID is selected from the group consisting of piroxicam, meloxicam, tenoxicam, droxicam, lornoxicam, phenylbutazone, or a derivative thereof. In an exemplary embodiment, the NSAID is an anthranilic acid derivative. In an exemplary embodiment, the NSAID is selected from the group consisting of mefenamic acid, meclofenamic acid, flufenamic acid, tolfenamic acid, or a derivative thereof. In an exemplary embodiment, the side-effect ameliorating agent is selected from the group consisting of phenazone, metamizole, and nabumetone, or a derivative thereof. In an exemplary embodiment, the side-effect ameliorating agent is an opiate. In an exemplary embodiment, the side-effect ameliorating agent is codeine, morphine, thebaine, or fentanyl. In an exemplary embodiment, the side-effect ameliorating agent is dihydrocodeine, oxymorphol, oxycodone, oxymorphone, or metopon.

### VIII. b6) Cytoprotective Agent

In an exemplary embodiment, the side-effect ameliorating agent is a cytoprotective agent. In an exemplary embodiment, the side-effect ameliorating agent is an aminothiol compound. In an exemplary embodiment, the side-effect ameliorating agent is amifostine. In an exemplary embodiment, the side-effect ameliorating agent is bleomycin, dexrazoxane, or coenzyme M.

### VIII. b7) Vasopressor Agent

In an exemplary embodiment, the side-effect ameliorating agent is a vasopressor agent. In an exemplary embodiment, the vasopressor agent is selected from norepinephrine, phenylephrine, epinephrine, ephedrine, dopamine, vasopressin, or a combination thereof. In an exemplary embodiment, the vasopressor agent is selected from dobutamine, midodrine, amezinium, or a combination thereof.

### VIII. b8) Anticonvulsant Agent

In an exemplary embodiment, the side-effect ameliorating agent is an anticonvulsant agent. In an exemplary embodiment, the anticonvulsant is an aldehyde. In an exemplary embodiment, the aldehyde is paraldehyde. In an exemplary embodiment, the anticonvulsant is an aromatic allylic alcohol. In an exemplary embodiment, the aromatic allylic alcohol is stiripentol. In an exemplary embodiment, the anticonvulsant is a barbiturate. In an exemplary embodiment, the barbiturate is phenobarbital, primidone, methylphenobarbital, or barbexaclone. In an exemplary embodiment, the anticonvulsant is a benzodiazepine. In an exemplary embodiment, the benzodiazepine is clobazam, clonazepam, clorazepate, diazepam, midazolam, lorazepam, nitrazepam, temazepam, and nimetazepam. In an exemplary embodiment, the anticonvulsant is a carboxamide. In an exemplary embodiment, the carboxamide is carbamazepine, oxcarbazepine, or eslicarbazepine acetate. In an exemplary embodiment, the anticonvulsant is a fatty acid. In an exemplary embodiment, the fatty acid is a valproate. In an exemplary embodiment, the valproate is valproic acid, sodium valproate, or divalproex sodium. In an exemplary embodiment, the valproate is vigabatrin, progabide, and tiagabine. In an exemplary embodiment, the anticonvulsant is a fructose derivative. In an exemplary embodiment, the fructose derivative is topiramate. In an exemplary embodiment, the anticonvulsant is a GABA analog. In an exemplary embodiment, the GABA analog is gabapentin or pregabalin. In an exemplary embodiment, the anticonvulsant is a hydantoin. In an exemplary embodiment, the hydantoin is ethotoin, phenytoin, mephenytoin, or fosphenytoin. In an exemplary embodiment, the anticonvulsant is an oxazolidinedione. In an exemplary embodiment, the oxazolidinedione is paramethadione, trimethadione, and ethadione. In an exemplary embodiment, the anticonvulsant is a propionate. In an exemplary embodiment, the anticonvulsant is a pyrimidinedione. In an exemplary embodiment, the anticonvulsant is a pyrrolidine. In an exemplary embodiment, the pyrrolidine is brivaracetam, etiracetam, levetiracetam, or seletracetam. In an exemplary embodiment, the anticonvulsant is levetiracetam. In an exemplary embodiment, the anticonvulsant is a succinimide. In an exemplary embodiment, the succinimide is ethosuximide, phensuximide, mesuximide. In an exemplary embodiment, the anticonvulsant is a sulfonamide. In an exemplary embodiment, the succinimide is acetazolamide, sultiame, methazolamide, and zonisamide. In an exemplary embodiment, the anticonvulsant is a triazine. In an exemplary embodiment, the triazine is lamotrigine. In an exemplary embodiment, the anticonvulsant is a urea. In an exemplary embodiment, the urea is pheneturide or phenacemide. In an exemplary embodiment, the anticonvulsant is a valproylamide. In an exemplary embodiment, the anticonvulsant is a valproylamide. In an exemplary embodiment, the valproylamide is valpromide or valnoctamide. In an exemplary embodiment, the anticonvulsant is perampanel, stiripentol, or pyridoxine.

### VIII. b9) TNFα Inhibitor

In an exemplary embodiment, the side-effect ameliorating agent is an anti-inflammatory agent. In an exemplary embodiment, the side-effect ameliorating agent is a TNF-α inhibitor. In an exemplary embodiment, the TNF-α inhibitor is an antibody. Examples of an anti-TNFα antibody molecule such as, infliximab (Remicade®), adalimumab (Humira®), certolizumab pegol (Cimzia®), and golimumab (Simponi®). Another example of a TNFα inhibitor is a fusion protein such as entanercept (Enbrel®). In an exemplary embodiment, the TNF-α inhibitor is a small molecule. Small molecule inhibitor of TNFα include, but are not limited to, xanthine derivatives (e.g. pentoxifylline) and bupropion.

### VIII. b10) IL6 Inhibitor

In an exemplary embodiment, the side-effect ameliorating agent is an anti-inflammatory agent. In an exemplary embodiment, the side-effect ameliorating agent is a IL-6 inhibitor. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab.

The methods described herein can comprise administering a bispecific anti-CD123×anti-CD3 antibody (e.g., XmAb14045) described herein to a subject and further administering one or more agents to manage elevated levels of a soluble factor resulting from treatment with a bispecific anti-CD123×anti-CD3 antibody (e.g., XmAb14045). In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8,

IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and inhibitor of IL-1R, and an inhibitor of IL-6. An example of an IL-1R based inhibitor is anakinra.

In an exemplary embodiment, the side-effect ameliorating agent is one that reduces an immune-mediated side effect. Exemplary immune-mediated side effects include, but are not limited to pneumonitis, colitis, hepatitis, nephritis and renal disfunction, hypothyroidism, hyperthyroidism, and endocrinopathies (e.g., hypophysitis, Type 1 diabetes mellitus and thyroid disorders such as hypothyroidism and hyperthyroidism). In one embodiment, the side-effect ameliorating agent reduces embryofetal toxicity.

### VIII. c) Exemplary Combinations

Combination with One Other Therapeutic Agent

In an exemplary embodiment, a bispecific anti-CD123×anti-CD3 antibody (e.g., XmAb14045) is administered to the subject in combination with one other therapeutic agent. In an exemplary embodiment, a bispecific anti-CD123×anti-CD3 antibody (e.g., XmAb14045) is administered in combination with one other anti-cancer agent. In an exemplary embodiment, a bispecific anti-CD123×anti-CD3 antibody (e.g., XmAb14045) is administered in combination with a side-effect ameliorating agent. In an exemplary embodiment, a bispecific anti-CD123×anti-CD3 antibody (e.g., XmAb14045) is administered to the subject in combination with one other anti-cancer agent. In an exemplary embodiment, a bispecific anti-CD123×anti-CD3 antibody (e.g., XmAb14045) is administered to the subject in combination with one other anti-cancer agent, which is radiation. In an exemplary embodiment, a bispecific anti-CD123×anti-CD3 antibody (e.g., XmAb14045) is administered to the subject in combination with one other anti-cancer agent.

In an exemplary embodiment, a bispecific anti-CD123×anti-CD3 antibody (e.g., XmAb14045) is administered to the subject in combination with one other anti-cancer agent, which is a chemotherapeutic. In an exemplary embodiment, a bispecific anti-CD123×anti-CD3 antibody (e.g., XmAb14045) is administered to the subject in combination with one other chemotherapeutic, which is a pyrimidine analog. In an exemplary embodiment, a bispecific anti-CD123×anti-CD3 antibody (e.g., XmAb14045) is administered to the subject in combination with one other chemotherapeutic, which is cytarabine. In an exemplary embodiment, a bispecific anti-CD123×anti-CD3 antibody (e.g., XmAb14045) is administered to the subject in combination with one other chemotherapeutic, which is an anthracycline. In an exemplary embodiment, a bispecific anti-CD123×anti-CD3 antibody (e.g., XmAb14045) is administered to the subject in combination with one other chemotherapeutic, which is idarubicin. In an exemplary embodiment, a bispecific anti-CD123×anti-CD3 antibody (e.g., XmAb14045) is administered to the subject in combination with one other chemotherapeutic, which is daunorubicin. In an exemplary embodiment, a bispecific anti-CD123×anti-CD3 antibody (e.g., XmAb14045) is administered to the subject in combination with one other chemotherapeutic, which is an anthracenedione. In an exemplary embodiment, a bispecific anti-CD123×anti-CD3 antibody (e.g., XmAb14045) is administered to the subject in combination with one other chemotherapeutic, which is gemtuzumab. In an exemplary embodiment, a bispecific anti-CD123×anti-CD3 antibody (e.g., XmAb14045) is administered to the subject in combination with one other chemotherapeutic, which is an FLT3 inhibitor.

In an exemplary embodiment, a bispecific anti-CD123×anti-CD3 antibody (e.g., XmAb14045) is administered to the subject in combination with one other chemotherapeutic, which is a topoisomerase inhibitor. In an exemplary embodiment, a bispecific anti-CD123×anti-CD3 antibody (e.g., XmAb14045) is administered to the subject in combination with one other chemotherapeutic, which is a topoisomerase II inhibitor. In an exemplary embodiment, a bispecific anti-CD123×anti-CD3 antibody (e.g., XmAb14045) is administered to the subject in combination with one other chemotherapeutic, which is etoposide. In an exemplary embodiment, a bispecific anti-CD123×anti-CD3 antibody (e.g., XmAb14045) is administered to the subject in combination with one other chemotherapeutic, which is mitoxantrone. In an exemplary embodiment, a bispecific anti-CD123×anti-CD3 antibody (e.g., XmAb14045) is administered to the subject in combination with one other chemotherapeutic, which is an adenosine analog. In an exemplary embodiment, a bispecific anti-CD123×anti-CD3 antibody (e.g., XmAb14045) is administered to the subject in combination with one other chemotherapeutic, which is fludarabine. In an exemplary embodiment, a bispecific anti-CD123×anti-CD3 antibody (e.g., XmAb14045) is administered to the subject in combination with one other chemotherapeutic, which is cladribine.

In an exemplary embodiment, a bispecific anti-CD123×anti-CD3 antibody (e.g., XmAb14045) is administered to the subject in combination with one other anti-cancer agent, which is an antibody. In an exemplary embodiment, a bispecific anti-CD123×anti-CD3 antibody (e.g., XmAb14045) is administered to the subject in combination with one other anti-cancer agent, which is a PDL2 inhibitor, a TIM3 inhibitor, a LAG3 inhibitor, a CTLA4 inhibitor, a TIGIT inhibitor, a BTLA inhibitor, a CD47 inhibitor, or a IDO inhibitor. In an exemplary embodiment, a bispecific anti-CD123×anti-CD3 antibody (e.g., XmAb14045) is administered to the subject in combination with one other anti-cancer agent, which is a PD1 inhibitor. In an exemplary embodiment, a bispecific anti-CD123×anti-CD3 antibody (e.g., XmAb14045) is administered to the subject in combination with one other anti-cancer agent, which is spartalizumab. In an exemplary embodiment, a bispecific anti-CD123×anti-CD3 antibody (e.g., XmAb14045) is administered to the subject in combination with one other anti-cancer agent, which is a PDL1 inhibitor.

Combination with Two Other Therapeutic Agents

In an exemplary embodiment, a bispecific anti-CD123×anti-CD3 antibody (e.g., XmAb14045) is administered in combination with two other therapeutic agents. In an exemplary embodiment, a bispecific anti-CD123×anti-CD3 antibody (e.g., XmAb14045) is administered in combination with two other therapeutic agents, wherein each of the two other therapeutic agents are side effect ameliorating agents. In an exemplary embodiment, a bispecific anti-CD123×anti-CD3 antibody (e.g., XmAb14045) is administered in combination with two other therapeutic agents, wherein each of the two other therapeutic agents are anti-cancer agents. In an exemplary embodiment, a bispecific anti-CD123×anti-CD3 antibody (e.g., XmAb14045) is administered in combination with two other therapeutic agents, wherein one of the other agents is an anti-cancer agent, and the other agent is a side effect ameliorating agent.

In an exemplary embodiment, a bispecific anti-CD123×anti-CD3 antibody (e.g., XmAb14045) is administered to the subject in combination with two other anti-cancer agents, one of which is a chemotherapeutic. In an exemplary embodiment, a bispecific anti-CD123×anti-CD3 antibody (e.g., XmAb14045) is administered to the subject in combination with two other anti-cancer agents, one of which is a pyrimidine analog. In an exemplary embodiment, a bispecific anti-CD123×anti-CD3 antibody (e.g., XmAb14045) is administered to the subject in combination with two other anti-cancer agents, one of which is cytarabine. In an exemplary embodiment, a bispecific anti-CD123×anti-CD3 antibody (e.g., XmAb14045) is administered to the subject in combination with two other anti-cancer agents, one of which is an anthracycline. In an exemplary embodiment, a bispecific anti-CD123×anti-CD3 antibody (e.g., XmAb14045) is administered to the subject in combination with one other chemotherapeutic, one of which is idarubicin. In an exemplary embodiment, a bispecific anti-CD123×anti-CD3 antibody (e.g., XmAb14045) is administered to the subject in combination with two other anti-cancer agents, one of which is daunorubicin. In an exemplary embodiment, a bispecific anti-CD123×anti-CD3 antibody (e.g., XmAb14045) is administered to the subject in combination with two other anti-cancer agents, one of which is an anthracenedione. In an exemplary embodiment, a bispecific anti-CD123×anti-CD3 antibody (e.g., XmAb14045) is administered to the subject in combination with two other anti-cancer agents, one of which is gemtuzumab. In an exemplary embodiment, a bispecific anti-CD123×anti-CD3 antibody (e.g., XmAb14045) is administered to the subject in combination with two other anti-cancer agents, one of which is an FLT3 inhibitor.

In an exemplary embodiment, a bispecific anti-CD123×anti-CD3 antibody (e.g., XmAb14045) is administered to the subject in combination with two other anti-cancer agents, one of which is a topoisomerase inhibitor. In an exemplary embodiment, a bispecific anti-CD123×anti-CD3 antibody (e.g., XmAb14045) is administered to the subject in combination with two other anti-cancer agents, one of which is a topoisomerase II inhibitor. In an exemplary embodiment, a bispecific anti-CD123×anti-CD3 antibody (e.g., XmAb14045) is administered to the subject in combination with two other anti-cancer agents, one of which is etoposide. In an exemplary embodiment, a bispecific anti-CD123×anti-CD3 antibody (e.g., XmAb14045) is administered to the subject in combination with two other anti-cancer agents, one of which is mitoxantrone. In an exemplary embodiment, a bispecific anti-CD123×anti-CD3 antibody (e.g., XmAb14045) is administered to the subject in combination with two other anti-cancer agents, one of which is an adenosine analog. In an exemplary embodiment, a bispecific anti-CD123×anti-CD3 antibody (e.g., XmAb14045) is administered to the subject in combination with two other anti-cancer agents, one of which is fludarabine. In an exemplary embodiment, a bispecific anti-CD123×anti-CD3 antibody (e.g., XmAb14045) is administered to the subject in combination with two other anti-cancer agents, one of which is cladribine. In an exemplary embodiment, a bispecific anti-CD123×anti-CD3 antibody (e.g., XmAb14045) is administered to the subject in combination with two other anti-cancer agents, one of which is cytarabine and the other is idarubicin. In an exemplary embodiment, a bispecific anti-CD123×anti-CD3 antibody (e.g., XmAb14045) is administered to the subject in combination with two other anti-cancer agents, one of which is cytarabine and the other is daunorubicin. In an exemplary embodiment, a bispecific anti-CD123×anti-CD3 antibody (e.g., XmAb14045) is administered to the subject in combination with two other anti-cancer agents, one of which is cytarabine and the other is gemtuzumab. In an exemplary embodiment, a bispecific anti-CD123×anti-CD3 antibody (e.g., XmAb14045) is administered to the subject in combination with two other anti-cancer agents, one of which is cytarabine and the other is midostaurin. In an exemplary embodiment, a bispecific anti-CD123×anti-CD3 antibody (e.g., XmAb14045) is administered to the subject in combination with two other anti-cancer agents, one of which is cytarabine and the other is etoposide. In an exemplary embodiment, a bispecific anti-CD123×anti-CD3 antibody (e.g., XmAb14045) is administered to the subject in combination with two other anti-cancer agents, one of which is cytarabine and the other is mitoxantrone. In an exemplary embodiment, a bispecific anti-CD123×anti-CD3 antibody (e.g., XmAb14045) is administered to the subject in combination with two other anti-cancer agents, one of which is cytarabine and the other is cladribine. In an exemplary embodiment, a bispecific anti-CD123×anti-CD3 antibody (e.g., XmAb14045) is administered to the subject in combination with two other anti-cancer agents, one of which is mitoxantrone and the other is cladribine. In an exemplary embodiment, a bispecific anti-CD123×anti-CD3 antibody (e.g., XmAb14045) is administered to the subject in combination with two other anti-cancer agents, one of which is mitoxantrone and the other is etoposide. In an exemplary embodiment, a bispecific anti-CD123×anti-CD3 antibody (e.g., XmAb14045) is administered to the subject in combination with two other anti-cancer agents, one of which is cytarabine and the other is fludarabine. In an exemplary embodiment, a bispecific anti-CD123×anti-CD3 antibody (e.g., XmAb14045) is administered to the subject in combination with two other anti-cancer agents, one of which is idarubicin and the other is fludarabine.

In an exemplary embodiment, a bispecific anti-CD123×anti-CD3 antibody (e.g., XmAb14045) is administered to the subject in combination with two other therapeutic agents, wherein one of these two other therapeutic agents is radiation. In an exemplary embodiment, a bispecific anti-CD123×anti-CD3 antibody (e.g., XmAb14045) is administered to the subject in combination with two other therapeutic agents, wherein one of these two other therapeutic agents is a chemotherapeutic. In an exemplary embodiment, a bispecific anti-CD123×anti-CD3 antibody (e.g., XmAb14045) is administered to the subject in combination with two other anti-cancer agents, which are independently selected from a PDL2 inhibitor, a TIM3 inhibitor, a LAG3 inhibitor, a CTLA4 inhibitor, a TIGIT inhibitor, a BTLA inhibitor, a CD47 inhibitor, and a IDO inhibitor. In an exemplary embodiment, a bispecific anti-CD123×anti-CD3 antibody (e.g., XmAb14045) is administered to the subject in combination with two other therapeutic agents, wherein one of these two other therapeutic agents is an antibody. In an exemplary embodiment, a bispecific anti-CD123×anti-CD3 antibody (e.g., XmAb14045) is administered to the subject in combination with two other therapeutic agents, wherein one of these two other therapeutic agents is a PD1 inhibitor. In an exemplary embodiment, a bispecific anti-CD123×anti-CD3 antibody (e.g., XmAb14045) is administered to the subject in combination with two other therapeutic agents, wherein one of these two other therapeutic agents is spartalizumab. In an exemplary embodiment, a bispecific anti-CD123×anti-CD3 antibody (e.g., XmAb14045) is administered to the subject in combination with two other therapeutic agents, wherein one of these two other therapeutic agents is a PDL1 inhibitor. In an exemplary embodiment, a bispecific anti-CD123×anti-CD3 antibody (e.g., XmAb14045) is administered to the subject in combination with two other therapeutic agents, wherein one of these two other therapeutic agents is a corticosteroid. In an exemplary embodiment, a bispecific anti-CD123×anti-CD3 antibody (e.g., XmAb14045) is administered to the subject in combination with two other therapeutic agents, wherein one of these two other therapeutic agents is a corticosteroid, and the other is a chemotherapeutic. In an exemplary embodiment, a bispecific anti-CD123×anti-CD3 antibody (e.g., XmAb14045) is administered to the subject in combination with two other therapeutic agents, wherein one of these two other therapeutic agents is a corticosteroid, and the other is an antibody. In an exemplary embodiment, a bispecific anti-CD123×anti-CD3 antibody (e.g., XmAb14045) is administered to the subject in combination with two other therapeutic agents, wherein one of these two other therapeutic agents is a corticosteroid, and the other is a PD1 inhibitor. In an exemplary embodiment, a bispecific anti-CD123×anti-CD3 antibody (e.g., XmAb14045) is administered to the subject in combination with two other therapeutic agents, wherein one of these two other therapeutic agents is a corticosteroid, and the other is a PDL1 inhibitor.

Combination with Three Other Therapeutic Agents

In an exemplary embodiment, a bispecific anti-CD123×anti-CD3 antibody (e.g., XmAb14045) is administered in combination with three other therapeutic agents. In an exemplary embodiment, a bispecific anti-CD123×anti-CD3 antibody (e.g., XmAb14045) is administered in combination with three other therapeutic agents, wherein each of the three other therapeutic agents are side effect ameliorating agents. In an exemplary embodiment, a bispecific anti-CD123×anti-CD3 antibody (e.g., XmAb14045) is administered in combination with three other therapeutic agents, wherein each of the three other therapeutic agents are anti-cancer agents. In an exemplary embodiment, a bispecific anti-CD123×anti-CD3 antibody (e.g., XmAb14045) is administered in combination with three other therapeutic agents, wherein two of the other therapeutic agents are anti-cancer agents, and the third other therapeutic agent is a side-effect ameliorating agent. In an exemplary embodiment, a bispecific anti-CD123×anti-CD3 antibody (e.g., XmAb14045) is administered in combination with three other therapeutic agents, wherein one of the other therapeutic agents is an anti-cancer agent, and the other two therapeutic agents are side-effect ameliorating agents.

In an exemplary embodiment, a bispecific anti-CD123×anti-CD3 antibody (e.g., XmAb14045) is administered to the subject in combination with three other therapeutic agents, wherein one of these three other therapeutic agents is radiation. In an exemplary embodiment, a bispecific anti-CD123×anti-CD3 antibody (e.g., XmAb14045) is administered to the subject in combination with three other therapeutic agents, wherein one of these three other therapeutic agents is a chemotherapeutic. In an exemplary embodiment, a bispecific anti-CD123×anti-CD3 antibody (e.g., XmAb14045) is administered to the subject in combination with three other anti-cancer agent, in which one of these anti-cancer agents is a PDL2 inhibitor, a TIM3 inhibitor, a LAG3 inhibitor, a CTLA4 inhibitor, a TIGIT inhibitor, a BTLA inhibitor, a CD47 inhibitor, or a IDO inhibitor. In an exemplary embodiment, a bispecific anti-CD123×anti-CD3 antibody (e.g., XmAb14045) is administered to the subject in combination with three other anti-cancer agent, in which two of these anti-cancer agents are independently selected from a PDL2 inhibitor, a TIM3 inhibitor, a LAG3 inhibitor, a CTLA4 inhibitor, a TIGIT inhibitor, a BTLA inhibitor, a CD47 inhibitor, or a IDO inhibitor. In an exemplary embodiment, a bispecific anti-CD123×anti-CD3 antibody (e.g., XmAb14045) is administered to the subject in combination with three other anti-cancer agent, in which each of these anti-cancer agents is independently selected from a PDL2 inhibitor, a TIM3 inhibitor, a LAG3 inhibitor, a CTLA4 inhibitor, a TIGIT inhibitor, a BTLA inhibitor, a CD47 inhibitor, or a IDO inhibitor. In an exemplary embodiment, a bispecific anti-CD123×anti-CD3 antibody (e.g., XmAb14045) is administered to the subject in combination with three other therapeutic agents, wherein one of these three other therapeutic agents is an antibody. In an exemplary embodiment, a bispecific anti-CD123×anti-CD3 antibody (e.g., XmAb14045) is administered to the subject in combination with three other therapeutic agents, wherein one of these three other therapeutic agents is a PD1 inhibitor. In an exemplary embodiment, a bispecific anti-CD123×anti-CD3 antibody (e.g., XmAb14045) is administered to the subject in combination with three other therapeutic agents, wherein one of these three other therapeutic agents is spartalizumab. In an exemplary embodiment, a bispecific anti-CD123×anti-CD3 antibody (e.g., XmAb14045) is administered to the subject in combination with three other therapeutic agents, wherein one of these three other therapeutic agents is a PDL1 inhibitor. In an exemplary embodiment, a bispecific anti-CD123×anti-CD3 antibody (e.g., XmAb14045) is administered to the subject in combination with three other therapeutic agents, wherein one of these three other therapeutic agents is a corticosteroid.

In an exemplary embodiment, a bispecific anti-CD123×anti-CD3 antibody (e.g., XmAb14045) is administered to the subject in combination with three other therapeutic agents, wherein the agents are mitoxantrone, etoposide, and cytarabine. In an exemplary embodiment, a bispecific anti-CD123×anti-CD3 antibody (e.g., XmAb14045) is administered to the subject in combination with three other therapeutic agents, wherein one of the agents is cytarabine. In an exemplary embodiment, a bispecific anti-CD123×anti-CD3 antibody (e.g., XmAb14045) is administered to the subject in combination with three other therapeutic agents, wherein the agents are daunorubicin, etoposide, and cytarabine.

In an exemplary embodiment, a bispecific anti-CD123×anti-CD3 antibody (e.g., XmAb14045) is administered to the subject in combination with a kinase inhibitor. In an exemplary embodiment, a bispecific anti-CD123×anti-CD3 antibody (e.g., XmAb14045) is administered to the subject in combination with imatinib. In an exemplary embodiment, a bispecific anti-CD123×anti-CD3 antibody (e.g., XmAb14045) is administered to the subject in combination with nilotinib or dasatinib or bosutinib. In an exemplary embodiment, a bispecific anti-CD123×anti-CD3 antibody (e.g., XmAb14045) is administered to the subject in combination with ponatinib or bosutinib. In an exemplary embodiment, for any of the combinations in this paragraph, a PD1 inhibitor is also part of the combination. In an exemplary embodiment, for any of the combinations in this paragraph, a PDL1 inhibitor is also part of the combination.

In an exemplary embodiment, a bispecific anti-CD123×anti-CD3 antibody (e.g., XmAb14045) is administered to the subject in combination with omacetaxine. In an exemplary embodiment, a bispecific anti-CD123×anti-CD3 antibody (e.g., XmAb14045) is administered to the subject in combination with omacetaxine and one kinase inhibitor. In an exemplary embodiment, a bispecific anti-CD123×anti-CD3 antibody (e.g., XmAb14045) is administered to the subject in combination with omacetaxine and two kinase inhibitors. In an exemplary embodiment, for any of the combinations in this paragraph, a PD1 inhibitor is also part of the combination. In an exemplary embodiment, for any of the combinations in this paragraph, a PDL1 inhibitor is also part of the combination.

In an exemplary embodiment, a bispecific anti-CD123×anti-CD3 antibody (e.g., XmAb14045) is administered to the subject in combination with three other therapeutic agents, wherein one is a corticosteroid and another is an PD1 inhibitor. In an exemplary embodiment, a bispecific anti-CD123×anti-CD3 antibody (e.g., XmAb14045) is administered to the subject in combination with three other therapeutic agents, wherein one is a corticosteroid and another is an PDL1 inhibitor. In an exemplary embodiment, a bispecific anti-CD123×anti-CD3 antibody (e.g., XmAb14045) is administered to the subject in combination with three other therapeutic agents, wherein one is a corticosteroid, another is Benadryl®, and the third is acetaminophen.

In an exemplary embodiment, the subject is administered one additional agent combination of a corticosteroid (e.g., dexamethasone, methylprednisolone, hydrocortisone) and Benadryl® and Tylenol®, wherein said corticosteroid, Benadryl® and Tylenol® are administered to the subject prior to the administration of the anti-CD123×anti-CD3 antibody (e.g., XmAb14045).

### Timing of Combination

In an exemplary embodiment, at least one of the other therapeutic agents is administered prior to the administration of the anti-CD123×anti-CD3 antibody (e.g., XmAb14045). In an exemplary embodiment, at least one of the other therapeutic agents is administered at the same time as the administration of the anti-CD123×anti-CD3 antibody (e.g., XmAb14045). In an exemplary embodiment, at least one of the other therapeutic agents is a corticosteroid, and this corticosteroid is administered prior to the administration of the anti-CD123×anti-CD3 antibody (e.g., XmAb14045).

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, and 2014/0288275, as well as PCT Publication WO2014/145806, as well as U.S. application Ser. Nos. 62/085,027, 14/952,714, and 15/141,350, all of which are expressly incorporated by reference in their entirety and particularly for the techniques outlined therein.

### Example 1

### XmAb14045 Treatment Plan

This is a multicenter, open-label, multi-dose, single-arm, Phase 1, dose-escalation study of XmAb14045. The dose of XmAb14045 will be administered IV over a 2-hr infusion period. Modifications of the dose infusion period may occur based on any observed infusion toxicity.

This study will be conducted in 2 sequential parts, Parts A and B.

Part A:

Human subjects will be enrolled in up to 8 consecutive dose cohorts (0.003, 0.01, 0.03, 0.075, 0.15, 0.3, 0.5, and 0.75 μg/kg) with initial accelerated titration for the first 3 cohorts. The first 3 cohorts will consist of 1 human subject each until there is evidence of a Grade 2 toxicity, and the remaining cohorts will enroll at least 3 human subjects each in a classic 3+3 dose escalation scheme. Human subjects will be admitted for 3 days for the first and fourth doses (and 2 days for the second dose, if admission is necessary to collect cytokine/inflammatory factors for the 8 hr postinfusion timepoint) for observation, PK, PD, and laboratory assessment. Within each ascending dose cohort (Cohorts 1A-8A), human subjects will be given XmAb14045 IV over 2 hr, once every 7 days, for a total of 4 doses in each 28-day cycle. The initial treatment period will include 2 cycles. After the MTD and/or RD dose is reached, the cohort may be expanded by up to an additional 12 human subjects to obtain additional safety data.

Part B:

An attempt will be made to escalate to higher doses for the second and subsequent drug infusions. Human subjects will be admitted for 3 days for the first and fourth dose as in Part A, but also for the escalated second dose (Day 8) for observation, PK, PD, and cytokine assessment.

The dose to be administered to the human subject for all cohorts will be calculated based on baseline (Day −1) weight measurement in kg. Following the first dose, subsequent doses will only be modified if the human subject's weight changes by more than 10% from the Day −1 weight at which point it will be recalculated using the current weight. For human subjects whose weight exceeds 100 kg, the dose of XmAb14045 will be calculated based on a weight of 100 kg and will NOT be calculated based upon the human subject's actual body weight.

A dose escalation schema will be employed in single dose level cohorts for Part A and sequentially increasing second and subsequent infusion dosing cohorts for Part B. Dose escalation will continue in both Parts A and B until the MTD and/or RD for further study has been identified or until a dose of 0.75 μg/kg has been reached, whichever comes first.

Human subjects will receive two 28-day cycles (8 weekly doses) of therapy. In the absence of unacceptable study drug-related toxicity, human subjects may receive additional cycles of therapy if there is clinical benefit (as assessed by the investigator). Doses will be administered on Days 1, 8, 15, and 22 of each cycle. Dosing may be delayed in the presence of drug-related toxicities. DLT determination and safety evaluation will occur after all relevant data is available through Day 22 of Cycle 1. If the MTD and/or RD are not reached, dose escalation to the next dose cohort will occur. Human subjects will be followed for at least 4 weeks after treatment is discontinued. Information regarding disease status will be collected by the investigational sites up to a final dose of XmAb14045, and followed by either clinic visit or telephone contact for an additional 6 months, or until the occurrence of death, stem cell transplantation, or disease progression requiring therapy (whichever comes first).

### Dose Escalation Scheme Part A

In Part A, dose level increases will initially proceed according to an accelerated titration design (see Table 2). This design allows for more efficient dose escalation while maintaining safety standards by implementing conservative triggers for cohort expansion during the accelerated escalation phase, and may limit the number of human subjects exposed to potentially sub-therapeutic doses of XmAb14045.

TABLE 2
Study Cohorts - Part A

| | | | Human |
|---|---|---|---|
| | Cohort | Planned Dose | subjects |
| Part A | 1A | 3 ng/kg (0.003 ug/kg) | 1 (+2+3) |
| | 2A | 10 ng/kg (0.01 ug/kg) | 1 (+2+3) |
| | 3A | 30 ng/kg (0.03 ug/kg) | 1 (+2+3) |
| | 4A | 75 ng/kg (0.075 ug/kg) | 3 (+3) |
| | 5A | 150 ng/kg (0.150 ug/kg) | 3 (+3) |
| | 6A | 300 ng/kg (0.3 ug/kg) | 3 (+3) |
| | 7A | 500 ng/kg (0.5 ug/kg) | 3 (+3) |
| | 8A | 750 ng/kg (0.75 ug/kg) | 3 (+3) |
| | Expansion-A | At MTD or recommended first | Up to 12 |
| | | infusion dose | |

During the initial accelerated dose escalation phase (Cohorts 1A, 2A, and 3A), dose escalation may occur after treatment of 1 human subject per cohort provided that there is no ≥Grade 2 toxicity during Cycle 1 and the human subject has met minimum safety assessment requirements (see Table 3). When a human subject experiences a ≥Grade 2 toxicity during the dose escalation safety assessment period, the accelerated escalation phase will end, the standard dose escalation phase will begin, and the cohort in which the event(s) occurred will be expanded to a total of at least 3 human subjects (2 additional human subjects will be enrolled).

TABLE 3
Dose Escalation Scheme
Accelerated Dose Escalation Phase

| | Number of Human | |
|---|---|---|
| Number of | Subjects Enrolled and | |
| Human Subjects | Assessable for Safety | |
| with at Least One | Following Four Doses | |
| Event ≥ Grade 2 | of XmAb14045 | Escalation Decision |
| 0 | 1 | Escalate to the next higher |
| | | dose level |
| 1 | 1 | Enroll 2 additional human |
| | | subjects on the same dose |
| | | level and revert to Standard |
| | | Dose Escalation (3 + 3) |
| | | design below. |

Standard Dose Escalation Phase

| | Number of Human | |
|---|---|---|
| Number of | Subjects Enrolled and | |
| Human Subjects | Assessable for Safety | |
| with at Least | Following Four Doses | |
| One DLT | of XmAb14045 | Escalation Decision |
| 0 | 3 | Escalate to the next higher |
| | | dose level |
| 1 | 3 | Enroll 3 additional human |
| | | subjects on the same dose |
| | | level |
| 1 | 6 | Escalate to the next higher |
| | | dose level |
| 2 | 3 or 6 | No dose escalation may |
| | | occur; MTD has been |
| | | surpassed. The next lower |
| | | dose level should be |
| | | expanded. |

DLT = dose-limiting toxicity; MTD = maximum tolerated dose

From this cohort forward (or beginning with Cohort 4A [0.075 μg/kg], whichever comes first) the standard 3+3 dose escalation rules will apply:

If zero of 3 human subjects have a DLT, then dose escalation to the next level will occur.

If 1 of 3 human subjects has a DLT, then the cohort will be further expanded to a total of 6 human subjects or until a second human subject in the cohort experiences a DLT. If there are no additional human subjects with a DLT, then dose escalation to the next higher dose level will occur.

The MTD is defined as the highest dose level at which no more than 1 human subject experiences DLT out of 6 human subjects assessable for toxicity at that dose level. Any cohort with 2 or more human subjects experiencing a DLT will have exceeded the MTD and there will be no further dose escalation. The dose level below the cohort at which 2 or more human subjects with DLT occurred will be expanded to at least 6 to delineate the MTD.

Before a dose-escalation decision can be reached, at least 1 human subject (in the accelerated dose escalation phase of the study) or 3 human subjects (in the standard escalation phase of the study) must meet all requirements for dose escalation safety assessment.

For the purpose of determining the incidence of DLT and defining the MTD and/or recommended dosing of XmAb14045 for future study, only human subjects who experience DLT and those with sufficient safety data/follow-up will be evaluated. Human subjects who complete 4 doses of XmAb14045 and undergo the planned safety evaluations through Day 22 will be considered to have sufficient safety data/follow-up. Human subjects who withdraw from study before completing Day 22 of treatment for reasons unrelated to study drug toxicity will be considered to have inadequate data to support dose escalation. In such cases, replacement human subjects will be enrolled to receive the same dose of XmAb14045 as the human subjects who withdraw prematurely.

Once the MTD (or RD for further study) is identified, the MTD/RD dose level may be further expanded up to an additional 12 human subjects (up to a total MTD/RD cohort of 18 human subjects) to further assess safety and PK.

The dose escalation scheme may be modified (e.g., smaller increases or decreases in dose level may be permitted, additional human subjects in a cohort may be enrolled, infusion duration and scheduling may be modified) based on the type and severity of toxicities observed in this trial, upon agreement of the DERC. Enrolling additional human subjects beyond 66 requires a protocol amendment.

### Dose Escalation Scheme—Part B

In Part B, the Day 1 dose will be fixed at the level determined in Part A. The second dose will be escalated and maintained for subsequent doses. Dosing cohorts will be defined relative to the MTD/RD determined in Part A.

TABLE 4
Study Cohorts-Part B

| | | | | | | Human |
|---|---|---|---|---|---|---|
| | Cohort | Day 1 | Day 8 | Day 15 | Day 22 | Subjects |
| Part B | −1B | X | X | X + 1 | X + 1 | 3 (+3) |
| | 1B | X | X + 1 | X + 1 | X + 1 | 3 (+3) |
| | 2B | X | X + 2 | X + 2 | X + 2 | 3 (+3) |
| | 3B | X | X + 3 | X + 3 | X + 3 | 3 (+3) |
| | 4B | X | X + 4 | X + 4 | X + 4 | 3 (+3) |
| | 5B | X | X + 5 | X + 5 | X + 5 | 3 (+3) |
| | 6B | X | X + 6 | X + 6 | X + 6 | 3 (+3) |
| | 7B | X | X + 7 | X + 7 | X + 7 | 3 (+3) |
| | Expansion-B | | At MTD or RD | | | Up to 12 |
| | | | cohort | | | |

MTD = maximum tolerated dose; RD = recommended dose; X = Part A MTD/RD

Dose escalation will proceed as described for the standard 3+3 scheme noted in Part A and with the same dosing levels (0.003, 0.01, 0.03, 0.075, 0.15, 0.3, 0.5, and 0.75 μg/kg) however the Day 1 infusion dose will always be the MTD/RD determined in Part A (denoted as “X” in Table 4). Dose escalation on each Part B cohort will be based on this starting point so for example if the MTD/RD from Part A is 0.03 μg/kg, the first infusion in Cohort 1B will be 0.03 μg/kg and the second and subsequent infusions will be at 0.075 μg/kg (i.e. X+1).

A minimum of 3 human subjects will be enrolled in each cohort. As in Part A, no two human subjects will start treatment with XmAb14045 on the same day. If all 3 human subjects tolerate a cohort without experiencing DLT (and the DERC agrees), enrollment will begin on the next higher cohort. If at any time through Day 22 a DLT occurs, 3 additional human subjects will be added to the cohort. If there is an additional DLT among the 6 human subjects on the cohort, the previous dosing cohort will be expanded to 6 to establish a MTD and/or RD. If this occurs on cohort 1B, the next 3 human subjects will be enrolled on cohort −1B. If there are no further DLTs among the 3 additional human subjects, another 3 human subjects will be added to the cohort. If there is an additional DLT, then the MTD/RD and schedule established in Part A will be recommended for further study.

### Example 2

### In Vitro Antitumor Efficacy

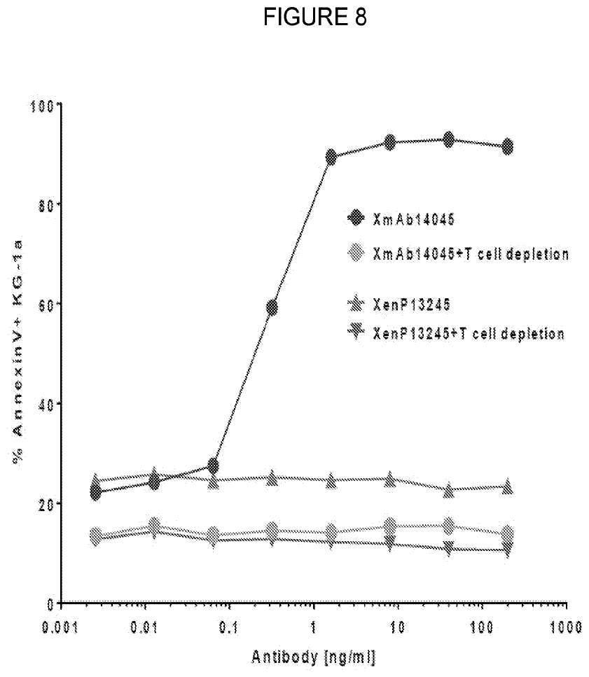

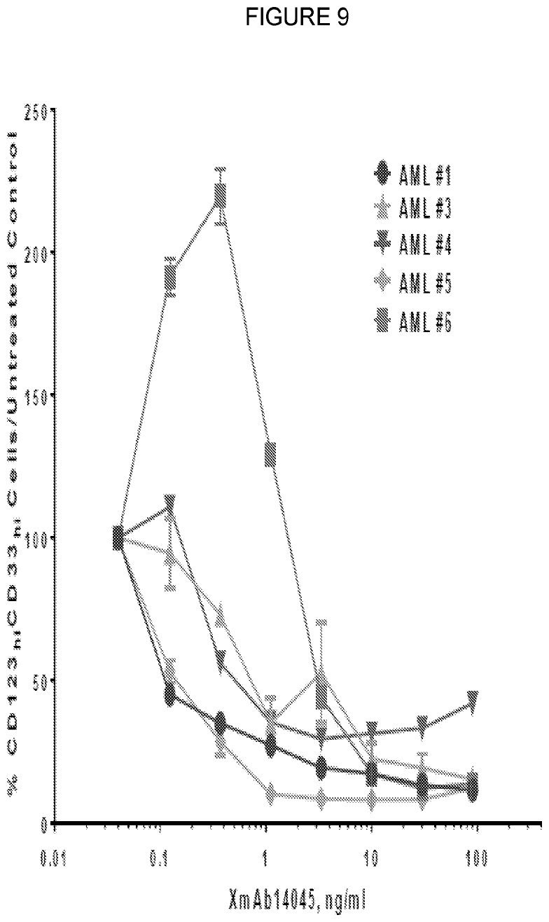

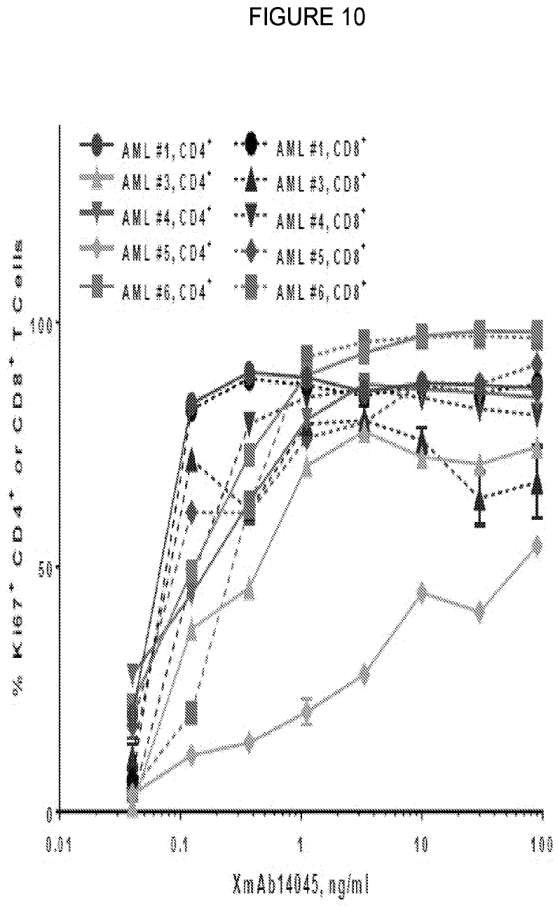

T cell-dependent cytotoxicity of XmAb14045 against CD123-positive (KG1a and Kasumi-3) and CD123-negative (Ramos) cell lines was examined using purified PBMC or T cell-depleted PBMC as effector cells. In addition, T cell activation was assessed by quantifying CD69 induction (a marker of lymphocyte activation) on both CD4+ and CD8+ T cells. XENP13245, an anti-RSV×anti-CD3 bsAb, was used as a control. XmAb14045, but not XENP13245, showed robust and potent killing of the CD123⁺ KG-1a (EC₅₀ of 0.28 ng/mL; see FIG. 8) and Kasumi-3 (EC₅₀ of 0.01 ng/mL) cell lines when supplied with human PBMC as an effector population along with robust CD69 induction in both CD4⁺ and CD8⁺ T cells. However, when T cells were depleted from PBMC (FIG. 8), XmAb14045 failed to induce killing or induce CD69 expression on T cells. XmAb14045 did not induce cytotoxicity of the CD123⁻ Ramos B cell line or induce T cell activation as measured by CD69 expression.

A series of studies was performed to evaluate the functionality of T-cells derived from AML human subject-derived PBMC. In particular, the ability of XmAb14045 to mediate RTCC towards various target populations found within, or added to, the AML samples was investigated. The target populations included: 1) a CD123ₕᵢCD33ₕᵢ population that arises in both AML PBMC and healthy PBMC upon incubation in culture for several days; 2) putative AML blast cells identified in the samples by flow cytometry; and 3) added KG1a AML cells. CD123-dependent T cell activation was measured by CD25 and Ki-67 upregulation on T cells. CD123-dependent target cell killing was monitored using annexin-V staining and by monitoring the reduction of counted blast cells.

Multiple AML human subject PBMC and normal PBMC samples were tested for XmAb14045-induced target cell killing and T cell activation. Both AML and normal PBMC contained CD123_{high} and CD33_{high} (CD123ₕᵢCD33ₕᵢ) cells; therefore, this population likely does not represent leukemic blast cells, but does serve as a useful surrogate target population. After 6 days incubation of PBMCs with XmAb14045, dose-dependent partial depletion of CD123ₕᵢCD33ₕᵢ, cells was induced in AML human subject-derived PBMC, accompanied by CD4⁺ and CD8⁺ T cell activation and proliferation.

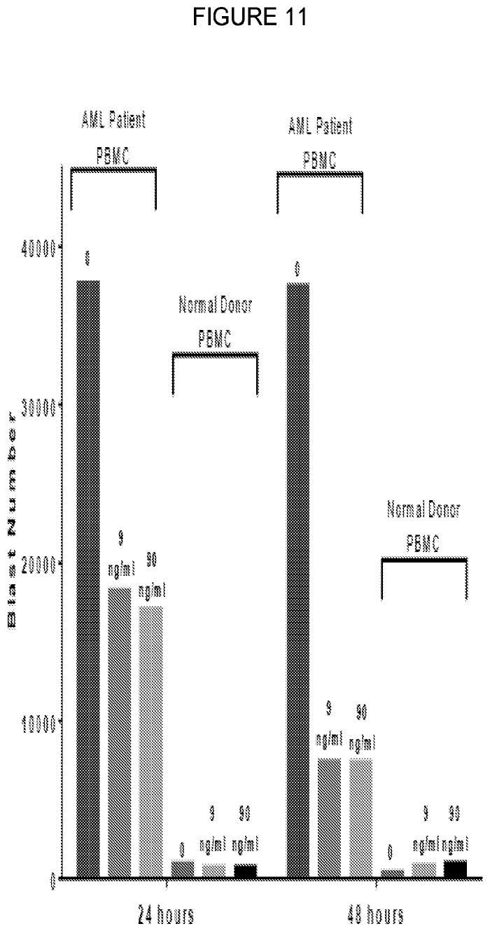

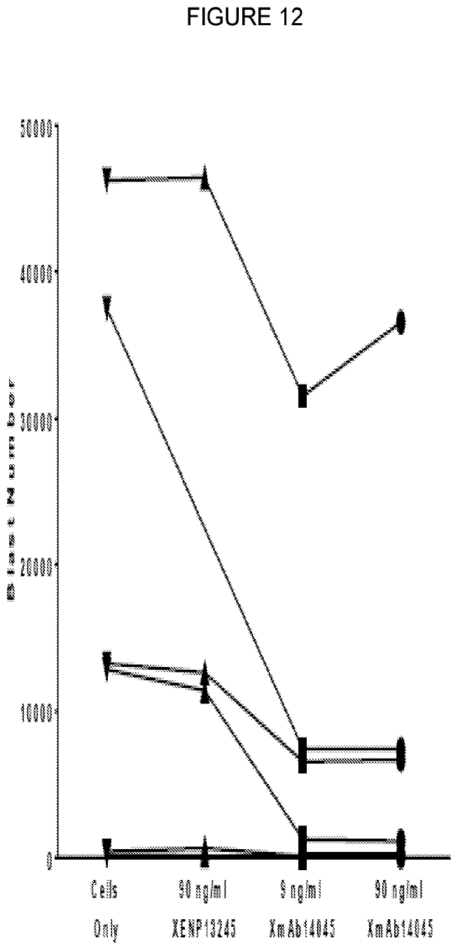

In a second set of studies, a modified staining process was used to detect leukemic blast cells in PBMC from a human subject with AML. AML PBMCs or PBMCs from a normal control donor were incubated for 24 or 48 hours with XmAb14045 at concentrations of 9 or 90 ng/mL and the putative blast cell number was obtained by flow cytometry. XmAb14045 reduced blast number by approximately 80% at 48 hours (FIG. 11). As expected, no blasts were seen in the normal donor PBMCs. This result was extended by assessing a total of 6 AML human subjects. XmAb14045 at concentrations of 9 or 90 ng/mL or XENP13245 (anti-RSV×anti-CD3) as a negative control. XmAb14045 depleted this putative blast cell population in AML PBMC at 48 hours by approximately 20% to 90%, with no apparent dependence on the number of target cells or T cells in the samples (see FIG. 12). The depletion was again associated with activation and proliferation of T cells.

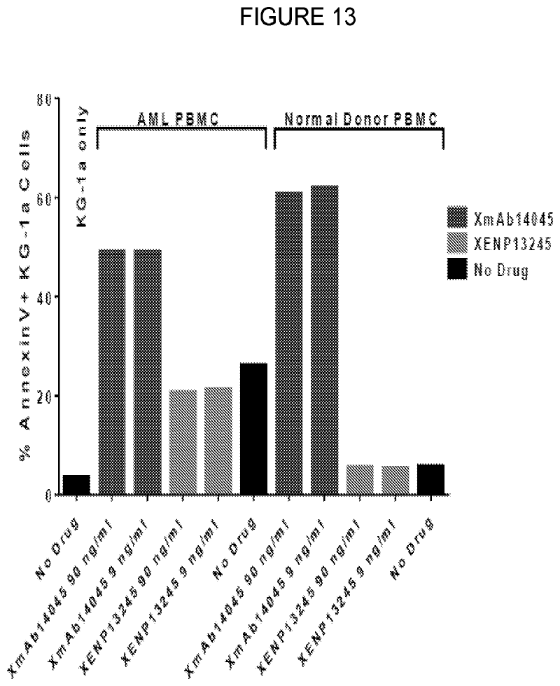

In a third set of studies, killing of an AML tumor cell line by AML human subject T cells was assessed. PBMC from one AML donor was mixed with the CD123-expressing cell line KG-1a in the presence of XmAb14045 for 48 hours (see FIG. 13). At 48 hours, XmAb14045 with AML human subject-derived PBMC induced robust apoptosis (approximately 50% annexin-V positivity), albeit still slightly lower than that induced with normal PBMC. XmAb14045 again induced robust proliferation of both AML human subject and healthy donor CD4⁺ and CD8⁺ T cells.

In summary, XmAb14045 induced allogeneic CD123⁺ KG-1a tumor cell killing by both AML human subject-derived and normal PBMC. More importantly, XmAb14045 induced autologous leukemic blast cell killing in PBMC from multiple AML human subject samples, suggesting that it could also stimulate depletion of leukemic blast cells in AML human subjects. Additionally, XmAb14045 in the presence of CD123⁺ target cells induced both CD4⁺ and CD8⁺ T cell activation in AML human subject and normal PBMC, indicating that AML human subject T cells are fully functional and capable of responding to XmAb14045.

### Example 3

### Antitumor Activity in a Mouse AML Xenograft Model

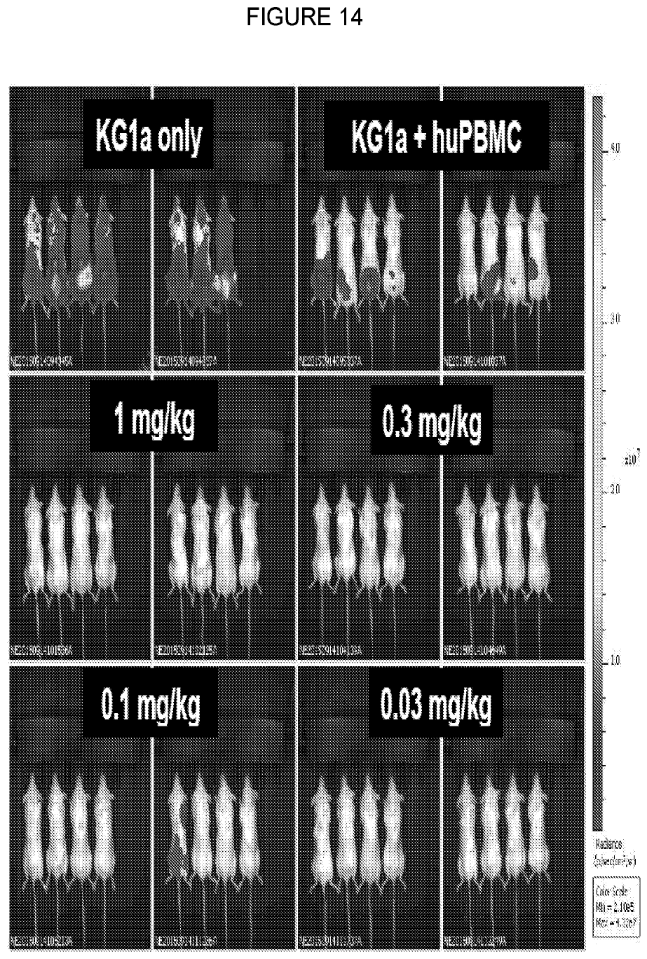

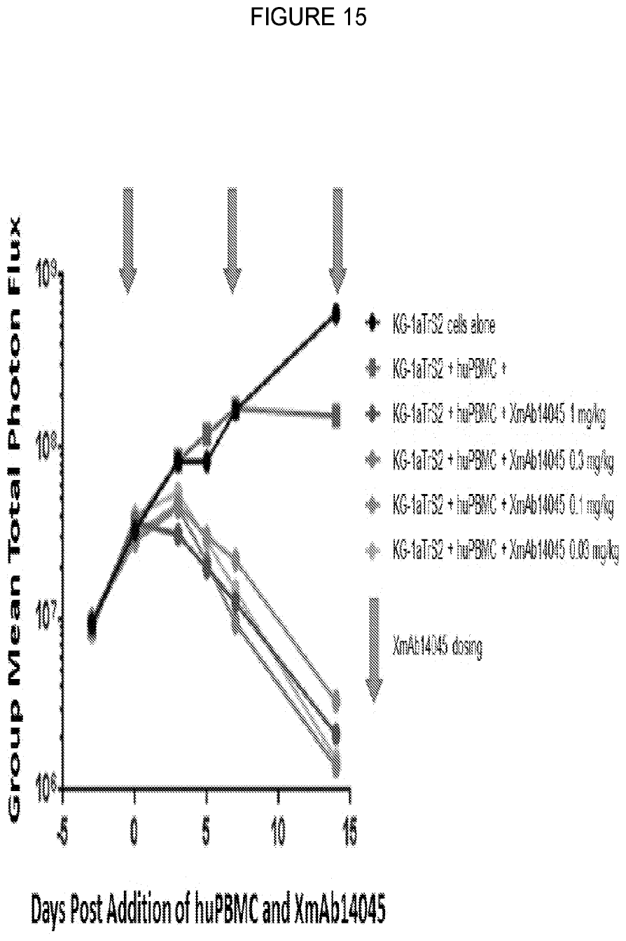

The anti-tumor activity of varying doses of XmAb14045 was examined in NSG mice that were engrafted systemically with KG1aTrS2 cells and normal human PBMCs. KG1aTrS2 cells are derived from the AML cell line KG1a, and have been engineered to express luciferase to allow quantification of tumor burden. Mice received 1×10⁶ KG1aTrS2 cells IV on Day 0. Twenty-two days after injection of KG1aTrS2 cells, mice were engrafted intraperitoneally (IP) with 10×10⁶ PBMC and were treated with 0.03, 0.1, 0.3 or 1.0 mg/kg of XmAb14045 or vehicle once a week for 3 consecutive weeks. Tumor burden was monitored throughout the study by in vivo imaging (FIG. 14). As shown in FIG. 14 and FIG. 15, mice receiving KG1a cells alone or KG1a cells plus PBMC displayed steadily increasing AML burden over time. In contrast, all tested dose levels of XmAb14045 began reducing tumor burden approximately 3 days after the initial dose, ultimately reducing burden by approximately 3 orders of magnitude relative to the KG1a-only control group, and significantly compared to the KG1a-plus-huPBMC group. No significant differences in anti-tumor activity were observed across the XmAb14045 dose range, suggesting that even lower doses would likely still exhibit anti-tumor activity.

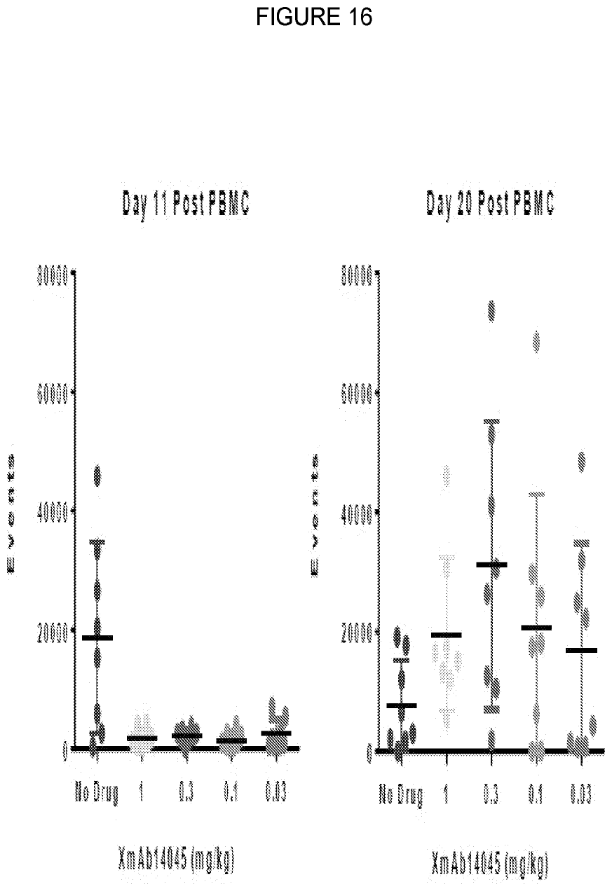

Peripheral blood samples were analyzed by flow cytometry. At Day 11, CD4⁺ and CD8⁺ T cell numbers were decreased in the treated mice compared to control, but by Day 20 this difference was no longer apparent, with a trend toward an increase in T cell counts, suggesting T cell activation and expansion mediated by XmAb14045 (FIG. 16). As another sign of T cell activation, PD1 expression was consistently higher on T cell samples from the XmAb14045-treated groups. However, it is unclear from this study whether the increase in PD1 expression interferes with the activity of XmAb14045.

What is claimed is:

1. A method for treating a CD123-expressing cancer in a human subject, comprising:
administering to the human subject having the CD123-expressing cancer an intravenous dose of a bispecific anti-CD123×anti-CD3 antibody in combination with at least one other therapeutic agent, for a time period sufficient to treat the CD123-expressing cancer,
wherein at least one of the other therapeutic agents is selected from the group consisting of PD1 inhibitors, PDL1 inhibitors, PDL2 inhibitors, TIM3 inhibitors, LAG3 inhibitors, CTLA4 inhibitors, TIGIT inhibitors, BTLA inhibitors, CD47 inhibitors, IDO inhibitors, GITR agonists, and ICOS agonists,
thereby treating said CD123-expressing cancer.

2. The method of claim 1, wherein the bispecific anti-CD123×anti-CD3 antibody comprises:
a) a first monomer comprising SEQ ID NO: 1;
b) a second monomer comprising SEQ ID NO: 2; and
c) a light chain comprising SEQ ID NO: 3.

3. The method of claim 1, wherein the bispecific anti-CD123×anti-CD3 antibody comprises:
a) an anti-CD123 variable heavy (VH) domain comprising SEQ ID NO: 19;
b) an anti-CD123 variable light (VL) domain comprising SEQ ID NO: 20;
c) an anti-CD3 variable heavy (VH) domain comprising SEQ ID NO: 21; and
d) an anti-CD3 variable light (VL) domain comprising SEQ ID NO: 22.

4. The method of claim 1, wherein the bispecific anti-CD123×anti-CD3 antibody comprises:
a) an anti-CD3 VH domain comprising a VHCDR1 comprising SEQ ID NO: 23, a VHCDR2 comprising SEQ ID NO: 24 and a VHCDR3 comprising SEQ ID NO: 25;
b) an anti-CD3 VL domain comprising a VLCDR1 comprising SEQ ID NO: 26, a VLCDR2 comprising SEQ ID NO: 27 and a VLCDR3 comprising SEQ ID NO: 28;
c) an anti-CD123 VH domain comprising a VHCDR1 comprising SEQ ID NO: 29, a VHCDR2 comprising SEQ ID NO: 30 and a VHCDR3 comprising SEQ ID NO: 31;
d) an anti-CD123 VL domain comprising a VLCDR1 comprising SEQ ID NO: 32, a VLCDR2 comprising SEQ ID NO: 33 and a VLCDR3 comprising SEQ ID NO: 34.

5. The method of claim 1, wherein the bispecific anti-CD123×anti-CD3 antibody is XmAb14045.

6. The method of claim 1, wherein the at least one of the other therapeutic agents is a PD1 inhibitor.

7. The method of claim 6, wherein the PD1 inhibitor is an anti-PD1 antibody.

8. The method of claim 7, wherein the anti-PD1 antibody is selected from the group consisting of nivolumab, pembrolizumab, pidilizumab, spartalizumab, JNJ-63723283, TSR-042, cemiplimab, AMP-224, MEDI0680, MGA012, MGD013, MGD019, SHR-1210, GLS-010, JS001, tislelizumab, sintilimab, CX-188, and CS1003.

9. The method of claim 7, wherein the anti-PD1 antibody is selected from the group consisting of nivolumab, pembrolizumab, and pidilizumab.

10. The method of claim 7, wherein the anti-PD1 antibody is spartalizumab.

11. The method of claim 1, wherein the at least one of the other therapeutic agents is a PDL1 inhibitor.

12. The method of claim 11, wherein the PDL1 inhibitor is an anti-PDL1 antibody.

13. The method of claim 12, wherein the anti-PDL1 antibody is selected from the group consisting of atezolizumab, avelumab, durvalumab, FAZ053, LY3300054, ABBV-181, MSB2311, BMS-936559, CS1001, KNO35, CA-327, CX-072, M7824, HTI-1316, and JS003.

14. The method of claim 1, wherein the at least one other therapeutic agent further comprises a chemotherapeutic.

15. The method of claim 14, wherein said chemotherapeutic is selected from the group consisting of alkylating agents, anti-metabolites, kinase inhibitors, proteasome inhibitors, vinca alkaloids, anthracyclines, antitumor antibiotics, aromatase inhibitors, topoisomerase inhibitors, mTOR inhibitors, retinoids, and combinations thereof.

16. The method of claim 1, wherein the at least one other therapeutic agent further comprises a side-effect ameliorating agent.

17. The method of claim 16, wherein said side-effect ameliorating agent is selected from the group consisting of: a steroid, an antihistamine, anti-allergic agents, antinausea agents, analgesic agent, antipyretic agent, cytoprotective agents, vasopressor agents, anticonvulsant agent, TNFα inhibitor, IL6 inhibitor, and combinations thereof.

18. The method of claim 16, wherein said side-effect ameliorating agent is selected from the group consisting of corticosteroids, TNFα inhibitors, IL-1R inhibitors, and IL-6 inhibitors.

19. The method of claim 16, wherein said side-effect ameliorating agent is a combination of a corticosteroid, Benadryl® and Tylenol®, wherein said corticosteroid, Benadryl® and Tylenol® are administered to said human subject prior to the administration of said bispecific anti-CD123×anti-CD3 antibody.

20. The method of claim 1, wherein said CD123-expressing cancer is a hematologic cancer.

21. The method of claim 1, wherein said CD123-expressing cancer is leukemia.

22. The method of claim 21, wherein the leukemia is selected from the group consisting of acute myeloid leukemia (AML), chronic myeloid leukemia (CML), acute lymphocytic leukemia (ALL), and hairy cell leukemia (HCL).

23. The method of claim 22, wherein the leukemia is acute myeloid leukemia (AML).

24. The method of claim 22, wherein the leukemia is chronic myeloid leukemia (CML).

25. The method of claim 22, wherein the acute myeloid leukemia (AML) is blastic plasmacytoid dendritic cell neoplasm (BPDCN).

26. The method of claim 22, wherein the leukemia is acute lymphocytic leukemia (ALL), and the acute lymphocytic leukemia is B-cell acute lymphocytic leukemia (B-ALL).

27. The method of any preceding claims, wherein the intravenous dose is:
between about 2 ng/kg and about 4 ng/kg; or
between about 9 ng/kg and about 11 ng/kg; or
between about 25 ng/kg and about 35 ng/kg; or
between about 70 ng/kg and about 80 ng/kg; or
between about 75 ng/kg and about 750 ng/kg; or
between about 125 ng/kg and about 175 ng/kg; or
between about 275 ng/kg and about 325 ng/kg; or
between about 475 ng/kg and about 525 ng/kg; or
between about 725 ng/kg and about 775 ng/kg.

28. The method of any preceding claims, wherein the intravenous dose is administered to the human subject between about 1 hour and about 3 hours.

29. The method of any preceding claims, wherein the time period sufficient to treat the leukemia is between about 3 weeks and 9 weeks.

30. The method of any preceding claims, wherein the bispecific anti-CD123×anti-CD3 antibody and the at least one other therapeutic agent are administered concurrently.

31. The method of any preceding claims, wherein the administration of the at least one other therapeutic agent begins before the administration of the bispecific anti-CD123×anti-CD3 antibody.

32. The method of any preceding claims, further comprising, prior to the administering, assessing the weight of the human subject.
